(19) United States
(12) Patent Application Publication
(10) Pub. No.: US 2024/0210423 A1
(43) Pub. Date: Jun. 27, 2024

(54) DIAGNOSIS AND TREATMENT OF AUTISM SPECTRUM DISORDERS ASSOCIATED WITH ALTERED METABOLIC PATHWAYS

(71) Applicant: Stemina Biomarker Discovery, Inc., Madison, WI (US)

(72) Inventors: Alan M. Smith, Madison, WI (US); Daniel Braas, Madison, WI (US); Michael Ludwig, Middleton, WI (US); Elizabeth L.R. Donley, Madison, WI (US); Robert Burrier, Verona, WI (US)

(21) Appl. No.: 17/608,285

(22) PCT Filed: Apr. 28, 2020
(86) PCT No.: PCT/US2020/030205

§ 371 Date: Jun. 21, 2022

Related U.S. Application Data
(60) Provisional application No. 62/842,059, filed on May 2, 2019.

(51) Int. Cl. G01N 33/68

(52) U.S. Cl. CPC G01N 33/6893; G01N 33/6812; G01N 33/6848; G01N 2800/28

(57) ABSTRACT

The invention provides methods of diagnosing autism spectrum disorders (ASD) by identification of altered metabolic pathways in such subjects. By measuring concentrations of metabolites in a sample, such as a blood or plasma sample, from a subject, changes in the activity of specific metabolic pathways can be identified. In turn, ASD subjects can be classified based on metabolic defects. Thus, the methods allow healthcare professionals to provide patient-specific guidance on a course of treatment for individuals who have or are at risk of developing ASD.

### CROSS-REFERENCE TO RELATED APPLICATIONS

This application claims the benefit of, and priority to, U.S. Provisional Patent Application No. 62/842,059, filed May 2, 2019, the contents of which are incorporated by reference.

### FIELD OF THE INVENTION

The invention relates generally to methods of diagnosing and treating individuals with autism spectrum disorders.

### BACKGROUND

The prevalence of Autism Spectrum Disorders (ASD) is high and growing rapidly. According to a 2018 report from the Centers for Disease Control and Prevention (CDC), the incidence of ASD in children in the United States more than doubled from 1 in 125 in 2008 to 1 in 59 in 2018. ASD includes a range of neurodevelopmental disorders that affect social and communication skills. Raising children with ASD places huge demands on parents and school systems, and adults with ASD are often have difficulty developing social relationships, maintaining jobs, and performing daily tasks.

The underlying basis of ASD is poorly understood, making ASD difficult both to diagnose and to treat. Although certain risk factors, such as high parental age and gestational diabetes, are associated with ASD, specific causes have not been identified. For example, autism displays a strong heritability component, but most cases cannot be linked to individual mutations. Thus, ASD is thought to result from multiple mutations that have low penetrance. In addition, many mutations that are associated with autism are not inherited from a parental genome but appear to have occurred during embryonic development. Therefore, ASD cannot be reliably predicted at an early stage from genetic data alone. Moreover, because the molecular mechanisms of ASD are not known, drugs to treat them are lacking. Existing pharmacological approaches are limited to the use of psychoactive or anticonvulsant medications to treat symptoms, such as irritability, self-injury, aggression, and tantrums, associated with ASD. However, such drugs do not remedy the social and communication impairments at the core of ASD. Consequently, the tools to diagnose and treat ASD remain woefully inadequate even as increasing numbers of people are affected by these disorders.

### SUMMARY

The invention provides methods of diagnosing and treating individuals having or at risk of developing neurodevelopmental disorders, such as ASD, by identification of altered metabolic pathways in such individuals. The invention is based on the discovery that analysis of ratios of concentrations of metabolites in individuals having or at risk of developing ASD reveals alterations in specific metabolic pathways associated with ASD. Moreover, different metabolic pathways are altered in different sub-populations of ASD patients, so patients can be classified according to the type of metabolic dysregulation. By pinpointing specific metabolic defects, physicians can identify ASD patients even before abnormalities in speech and behavior are evident. In addition, by enabling identification of metabolic deficiencies, the methods of the invention provide guidance on interventions that will correct those deficiencies.

A critical factor to success in treatment of ASD is early intervention. The diagnostic methods of the invention enable detection of ASD much earlier than is possible with prior methods. For example, altered metabolic pathways can be detected shortly after birth or even in utero. Therefore, the methods allow initiation of treatment at an early stage to promote normal neurological development.

In an aspect, the invention provides methods of providing guidance for treating a subject that has or is at risk of developing a neurodevelopmental disorder. The methods include receiving results of an assay in which concentrations of two or more metabolites are measured in a sample from a subject that has or is at risk of developing a neurodevelopmental disorder, and based on the results, providing guidance for treating the subject that has or is suspected of having a neurodevelopmental disorder. The results include at least one ratio of concentrations of the metabolites, a reference level that provides an indication as to whether the ratio is imbalanced, and identification of a metabolic pathway that includes at least one of the metabolites,
The metabolic pathway may be an amine metabolic pathway, a metabolic pathway related to a gut microbiome, a mitochondrial energy homeostasis pathway, a neurotransmission pathway, a neurotransmitter synthesis pathway, a purine degradation pathway, or a reactive oxidative species metabolic pathway.

The neurodevelopmental order may be an autism spectrum disorder. For example, the neurodevelopmental disorder may be autism, Asperger syndrome, pervasive developmental disorder not otherwise specified (PDD-NOS), or childhood disintegrative disorder.

Either of the metabolites may be alanine, asparagine, aspartic acid, glycine, histidine, hypoxanthine, inosine, kynurenine, lactate, leucine, lysine, ornithine, phenylalanine, pyruvate, succinate, taurine, uric acid, xanthine, or α-ketoglutarate.

The ratio of concentrations of metabolites may be asparagine to glycine; glycine to phenylalanine; histidine to leucine; kynurenine to ornithine; lactate to alanine; lactate to phenylalanine; lysine to ornithine; xanthine to uric acid; α-ketoglutarate to alanine; or α-ketoglutarate to lactate. The ratio may be ethanolamine to (glutamate and kynurenine); glutamine to isoleucine; glutamine to leucine; glutamine to valine; glycine to asparagine; glycine to glutamate; glycine to isoleucine; glycine to leucine; glycine to lysine; glycine to phenylalanine; glycine to valine; hypoxanthine to uric acid; lactate to phenylalanine; ornithine to isoleucine; ornithine to kynurenine; ornithine to leucine; ornithine to lysine; ornithine to phenylalanine; ornithine to valine; pyruvic acid to phenylalanine; serine to isoleucine; serine to leucine; serine to valine; or xanthine to hydroxyproline.

The ratio of concentrations may be a group of ratios of a first metabolite, such as an amino-containing compound, to branched amino acids, in which the branched chain amino acid are isoleucine, leucine, or valine. For example, the group of ratios of concentrations may be (A) glutamine to isoleucine; glutamine to leucine; and glutamine to valine, (B) glycine to isoleucine; glycine to leucine; and glycine to valine, (C) ornithine to isoleucine; ornithine to leucine; and ornithine to valine, (D) serine to isoleucine; serine to leucine; and serine to valine, or (E) hypoxanthine to uric acid; and xanthine to uric acid. Other groups include ratios of concentrations in which the first analyte in each ratio is the same and the second analyte in each ratio is different, i.e., groups of the general formula X:A, X:B, X:C, etc. Such groups may include two, three, four, five, or more ratios. The second analytes in such groups may have a common feature or be members of a common class of compounds. For example, the second analytes in such groups may be branched chain amino acids, hydrophobic amino acids, polar amino acids, negatively charged amino acids, positively charged amino acids, or metabolites in a common metabolic pathway, e.g., the citric acid cycle or fatty acid oxidation.

In certain embodiments, the metabolic pathway is purine degradation, and the metabolites are two or more of hypoxanthine, inosine, taurine, uric acid, and xanthine. In certain embodiments, the metabolic pathway is purine degradation, and the ratio is xanthine to uric acid.

In certain embodiments, the metabolic pathway is a mitochondrial energy homeostasis pathway, and the metabolites are two or more of alanine, lactate, phenylalanine, pyruvate, succinate, and α-ketoglutarate. In certain embodiments, the metabolic pathway is a mitochondrial energy homeostasis pathway, and the ratio is α-ketoglutarate to alanine; α-ketoglutarate to lactate, lactate to alanine; or lactate to phenylalanine.

In certain embodiments, the metabolic pathway is an amine metabolic pathway, a neurotransmission pathway, or a neurotransmitter synthesis pathway, and the metabolites are two or more of asparagine, glycine, histidine, kynurenine, leucine, lysine, ornithine, and phenylalanine. In certain embodiments, the metabolic pathway is an amine metabolic pathway, a neurotransmission pathway, or a neurotransmitter synthesis pathway, and the ratio is asparagine to glycine; glycine to phenylalanine; histidine to leucine; kynurenine to ornithine; or lysine to ornithine.

The reference population may be a subset of autism spectrum disorder (ASD) subjects. The subset may include subjects that have an alteration in a metabolic pathway in comparison to other ASD subjects, typically developing subjects, or in both. In such embodiments, a similar metabolic alteration in the subject may indicate that the subject from whom the sample was obtained has or is likely to develop ASD, and the absence of such an alteration may indicate that the subject from whom the sample was obtained does not have or is not likely to develop ASD. The reference population may include typically developing subjects. In such embodiments, a metabolic similarity or lack of alteration between the subject and the reference may indicate that the subject from whom the sample was obtained does not have or is not likely to develop ASD, and metabolic dissimilarity or alteration may indicate that the subject from whom the sample was obtained has or is likely to develop ASD. The reference population may include subjects that have a non-ASD developmental disorder.

The sample may be a body fluid sample. For example, the body fluid may be blood, plasma, urine, sweat, tears, or saliva.

The method may include receiving the sample from the subject. The method may include performing the assay. The assay may include mass spectrometry.

The results may include additional data about the subject. The additional data may include the medical history of the subject, medical history of a family member of the subject, or genetic data from the subject.

The methods may include distinguishing whether a subject has an ASD and or a non-ASD developmental disorder. Thus, the methods may include comparing a metabolic profile or metabotype of the subject with the metabolic profile or metabotype of a reference population of subjects that have a non-ASD developmental disorder. In such embodiments, a similar metabolic profile or metabotype may indicate that the subject has or is likely to develop a non-ASD developmental disorder or that the subject does not have or is not likely to develop an ASD developmental disorder. Conversely, in such embodiments, a dissimilar metabolic profile or metabotype may indicate that the subject has or is likely to develop an ASD developmental disorder.

The guidance may include a recommendation for a dietary modification, a drug, a medical grade food, or a supplement for the subject. The dietary modification may include supplementation with a source of metabolites or amino acids. The dietary modification may include supplementation with specific amino acids. For example, the dietary modification may include supplementation with one or more branched chain amino acids, such as isoleucine, leucine, or valine. The dietary modification may include supplementation with a source of metabolites or amino acids that is substantially free of phenylalanine, such as glycomacropeptide. The dietary modification may include decreasing the intake of specific metabolites or amino acids, such as phenylalanine.

The guidance may include a recommendation that the subject consult with a specialist, such as a neurodevelopment specialist or nutritionist.

The guidance may be provided in a report. The report may contain additional information about the subject, such as age, sex, weight, height, genetic data, genomic data, and dietary preferences.

The subject may be a human. The test subject may be a child. For example, the test subject may be a child of less than about 18 years of age, less than about 16 years of age, less than about 14 years of age, less than about 13 years of age, less than about 12 years of age, less than about 10 years of age, less than about 9 years of age, less than about 8 years of age, a child of less than about 7 years of age, a child of less than about 6 years of age, a child of less than about 5 years of age, a child of less than about 4 years of age, a child of less than about 3 years of age, a child of less than about 2 years of age, a child of less than about 18 months of age, a child of less than about 12 months of age, a child of less than about 9 months of age, a child of less than about 6 months of age, or a child of less than about 3 months of age.

In another aspect, the invention provides methods of determining whether a test subject has or is at risk of developing a neurodevelopmental disorder. The methods include receiving a sample from a test subject; conducting a mass spectrometry analysis on the sample to generate mass spectral data; performing, via a computer, one or more algorithmic analyses on the mass spectral data to determine concentrations of two or more metabolites in the sample; generating, via the computer, a test ratio of the concentrations of the at least two metabolites in the sample from the test subject; identifying a metabolic pathway containing at least one of the metabolites; and outputting the test ratio to a report that includes a reference ratio of concentrations of the metabolites in one or more samples from one or more typically developing subjects and indicates if the metabolic pathway is altered in the test subject compared to typically developing subject.

The neurodevelopmental disorder may be an autism spectrum disorder. For example, the neurodevelopmental disorder may be autism, Asperger syndrome, pervasive developmental disorder not otherwise specified (PDD-NOS), or childhood disintegrative disorder.

The methods may include the use of multiple test ratios and multiple reference ratios. The test and reference ratios of concentrations of metabolites may be one or more of ethanolamine to (glutamate and kynurenine); glutamine to isoleucine; glutamine to leucine; glutamine to valine; glycine to Asparagine; glycine to glutamate; glycine to isoleucine; glycine to leucine; glycine to lysine; glycine to phenylalanine; glycine to valine; His to leucine; hypoxanthine to uric acid; lactic acid to phenylalanine; ornithine to isoleucine; ornithine to kynurenine; ornithine to leucine; ornithine to lysine; ornithine to phenylalanine; ornithine to valine; pyruvic acid to phenylalanine; serine to isoleucine; serine to leucine; serine to valine; xanthine to hydroxyproline; and xanthine to uric acid.

The ratio of concentrations may be a group of ratios of a first metabolite to branched amino acids, in which the branched chain amino acid are isoleucine, leucine, or valine. For example, the group of ratios of concentrations may be (A) glutamine to isoleucine; glutamine to leucine; and glutamine to valine, (B) glycine to isoleucine; glycine to leucine; and glycine to valine, (C) ornithine to isoleucine; ornithine to leucine; and ornithine to valine, (D) serine to isoleucine; serine to leucine; and serine to valine, or (E) hypoxanthine to uric acid; and xanthine to uric acid. Other groups include ratios of concentrations in which the first analyte in each ratio is the same and the second analyte in each ratio is different, i.e., groups of the general formula X:A, X:B, X:C, etc. Such groups may include two, three, four, five, or more ratios. The second analytes in such groups may have a common feature or be members of a common class of compounds. For example, the second analytes in such groups may be branched chain amino acids, hydrophobic amino acids, polar amino acids, negatively charged amino acids, positively charged amino acids, or metabolites in a common metabolic pathway, e.g., the citric acid cycle or fatty acid oxidation.

The reference ratio may be defined in relation to a subset of autism spectrum disorder (ASD) subjects. The subset may include subjects that have a ratio of concentrations of two or more metabolites that is different from the ratio of concentrations of the two or more metabolites in other ASD subjects, in typically developing subjects, or in both. The reference ratio may be representative of subjects in the subset. In such embodiments, a match between the ratio obtained from the sample and the reference ratio may indicate that the subject from whom the sample was obtained has or is likely to develop ASD, and a mismatch may indicate that the subject from whom the sample was obtained does not have or is not likely to develop ASD. Alternatively, the reference ratio may be representative of typically developing subjects. In such embodiments, a match between the ratio obtained from the sample and the reference ratio may indicate that the subject from whom the sample was obtained does not have or is not likely to develop ASD, and a mismatch may indicate that the subject from whom the sample was obtained has or is likely to develop ASD.

The reference subject or population may be selected to have one or more characteristics the same as, similar to, or different from, those of the test subject. For example, the reference subject or population may be the same as, similar to, or different from, the test subject in age, sex, weight, height, genetic profile, or genomic profile.

A reference ratio may be or include an average value or a range of values. Thus, a match may be present if the ratio of concentration of metabolites in a sample obtained from a subject (1) falls above or below a threshold defined by the reference ratio, (2) falls within a range defined by a reference ratio, or (3) is otherwise similar to the ratio by some quantitative measure, and a mismatch may be present if the ratio of concentration of metabolites in sample obtained from a subject (1) does not fall above or below a threshold defined by the reference ratio, (2) does not fall within a range defined by a reference ratio, or (3) is otherwise different from the ratio by some quantitative measure. Likewise, two ratios may be deemed similar to each other by the same criteria for determining matching ratios, and two ratios may be different from each other by the same criteria for determining mismatched ratios.

The methods may include identifying whether a subject has metabolic dysregulation. For example, a subject may be identified as having metabolic dysregulation if results indicate an imbalance in one or more of the following ratios of concentrations: ethanolamine to (glutamate and kynurenine); glutamine to isoleucine; glutamine to leucine; glutamine to valine; glycine to Asparagine; glycine to glutamate; glycine to isoleucine; glycine to leucine; glycine to lysine; glycine to phenylalanine; glycine to valine; histidine to leucine; hypoxanthine to uric acid; lactic acid to phenylalanine; ornithine to isoleucine; ornithine to kynurenine; ornithine to leucine; ornithine to lysine; ornithine to phenylalanine; ornithine to valine; pyruvic acid to phenylalanine; serine to isoleucine; serine to leucine; serine to valine; xanthine to hydroxyproline; and xanthine to uric acid.

The ratio of concentrations may be a group of ratios of a first metabolite, such as an amine-containing compound, to branched amino acids, in which the branched chain amino acid are isoleucine, leucine, or valine. For example, the group of ratios of concentrations may be (A) glutamine to isoleucine; glutamine to leucine; and glutamine to valine, (B) glycine to isoleucine; glycine to leucine; and glycine to valine, (C) ornithine to isoleucine; ornithine to leucine; and ornithine to valine, (D) serine to isoleucine; serine to leucine; and serine to valine, or (E) hypoxanthine to uric acid; and xanthine to uric acid. Other groups include ratios of concentrations in which the first analyte in each ratio is the same and the second analyte in each ratio is different, i.e., groups of the general formula X:A, X:B, X:C, etc. Such groups may include two, three, four, five, or more ratios. The second analytes in such groups may have a common feature or be members of a common class of compounds. For example, the second analytes in such groups may be branched chain amino acids, hydrophobic amino acids, polar amino acids, negatively charged amino acids, positively charged amino acids, or metabolites in a common metabolic pathway, e.g., the citric acid cycle or fatty acid oxidation.

The methods may include distinguishing whether a subject has an ASD and or a non-ASD developmental disorder. Thus, the methods may include comparing the ratio of concentrations of the two or more metabolites in the sample obtained from the subject with a ratio of concentrations of the two or more metabolites in samples from subjects that have a non-ASD development disorder. Thus, a ratio of concentrations of the two or more metabolites in the sample obtained from the subject that is different from, or does not match, a ratio of concentrations of the two or more metabolites in samples from subjects that have a non-ASD developmental disorder may indicate that the subject from whom the sample was obtained has or is likely to develop ASD and/or that the subject from whom the sample was obtained does not have or is not likely to develop a developmental disorder. Conversely, a ratio of concentrations of the two or more metabolites in the sample obtained from the subject that is similar to, or matches, a ratio of concentrations of the two or more metabolites in samples from subjects that have a non-ASD developmental disorders may indicate that the subject from whom the sample was obtained does not have or is not likely to develop ASD and/or that the subject from whom the sample was obtained has or is likely to develop a non-ASD developmental disorder. For example, the results may indicate that the subject has or is likely to develop an ASD developmental disorder if each of the ratios in one of the following groups of ratios indicates an imbalance: (A) glutamine to isoleucine; glutamine to leucine; and glutamine to valine, (B) glycine to isoleucine; glycine to leucine; and glycine to valine, (C) ornithine to isoleucine; ornithine to leucine; and ornithine to valine, (D) serine to isoleucine; serine to leucine; and serine to valine, or (E) hypoxanthine to uric acid; and xanthine to uric acid.

The report may indicate that the test subject has or is at risk of developing a neurodevelopmental disorder if the test ratio is imbalanced compared to the reference ratio. The report may indicate a likelihood or probability that the test subject will develop a neurodevelopmental disorder. The report may indicate a likelihood or probability that the test subject will develop a neurodevelopmental disorder if the test subject goes untreated. The report may indicate a likelihood or probability that the test subject will develop a neurodevelopmental disorder if the test subject undergoes a particular course of treatment, such as a dietary modification.

The report may include guidance for treating the subject. The guidance may include a recommendation for a dietary modification for the subject, such as one or more of the dietary modifications described above. The guidance may include a recommendation for a drug, a medical grade food, or a supplement.

The guidance may include a recommendation that the subject consult with a specialist, such as a neurodevelopment specialist or nutritionist.

The report may contain additional information about the subject, such as age, sex, weight, height, genetic data, genomic data, and dietary preferences.

The sample may be a body fluid sample. For example, the body fluid may be blood, plasma, urine, sweat, tears, or saliva.

### BRIEF DESCRIPTION OF THE DRAWINGS

FIG. 1 is an outline of computational procedures utilized to set diagnostic thresholds and to evaluate diagnostic performance

FIG. 2 is a heat map with hierarchical clustering dendrograms from pairwise Pearson correlations of metabolite abundances for the training set ASD subjects

FIG. 3 is a scatter plot of the training set's transformed amine concentration values.

FIG. 4 shows scatter plots of ratios of levels of glutamine to various branched chain amino acids (BCAAs) in subjects with Autism Spectrum Disorder (ASD) and in typically developing subjects (TYP).

FIG. 5 shows scatter plots of levels of individual amino acids in ASD subjects and TYP subjects.

FIG. 6 is a Venn diagram of metabotype-positive subjects identified by the three ratios used for AADM_{glutamine}.

FIG. 7 shows scatter plots of ratios of levels of glycine to various branched chain amino acids in ASD subjects and TYP subjects.

FIG. 8 shows scatter plots of levels of individual amino acids in ASD subjects and TYP subjects.

FIG. 9 is a Venn diagram of metabotype-positive subjects identified by the three ratios used for AADM_{glycine}.

FIG. 10 shows scatter plots of ratios of levels of ornithine to various branched chain amino acids in ASD subjects and TYP subjects.

FIG. 11 shows scatter plots of levels of individual amino acids in ASD subjects and TYP subjects.

FIG. 12 is a Venn diagram of metabotype-positive subjects identified by the three ratios used for AADMₒᵣₙᵢₜₕᵢₙₑ.

FIG. 13 shows scatter plots of ratios of levels of alanine to various branched chain amino acids in ASD subjects and TYP subjects.

FIG. 14 shows scatter plots of levels of individual amino acids in ASD subjects and TYP subjects.

FIG. 15 is a Venn diagram of metabotype-positive subjects identified by the three ratios used for AADMₐₗₐₙᵢₙₑ.

FIG. 16 shows scatter plots of ratios of levels of homoserine to various branched chain amino acids in ASD subjects and TYP subjects.

FIG. 17 shows scatter plots of levels of individual amino acids in ASD subjects and TYP subjects. Red points represent AADMₕₒₘₒₛₑᵣᵢₙₑ positive subjects, and black points represent AADMₕₒₘₒₛₑᵣᵢₙₑ negative subjects.

FIG. 18 is a Venn diagram of metabotype-positive subjects identified by the three ratios used for AADMₕₒₘₒₛₑᵣᵢₙₑ.

FIG. 19 shows scatter plots of ratios of levels of serine to various branched chain amino acids in ASD subjects and TYP subjects.

FIG. 20 shows scatter plots of levels of individual amino acids in ASD subjects and TYP subjects.

FIG. 21 is a Venn diagram of metabotype-positive subjects identified by the three ratios used for AADMₛₑᵣᵢₙₑ.

FIG. 22 shows scatter plots of ratios of levels of 4-hydroxyproline to various branched chain amino acids in ASD subjects and TYP subjects.

FIG. 23 shows scatter plots of levels of individual amino acids in ASD subjects and TYP subjects.

FIG. 24 is a Venn diagram of metabotype-positive subjects identified by the three ratios used for AADM_{hydroxproline}.

FIG. 25 shows a Venn diagram of the 92 AADMₜₒₜₐₗ subjects identified by each of the AADMs.

FIG. 26 is graph showing the principal comment analysis of the metabolite ratios used in the metabolic signature of the reproducible AADMs creating the AADMₜₒₜₐₗ estimates in the CAMP study population.

FIG. 27 shows scatter plots of the ratios of levels of metabolites and levels of individual metabolites utilized in identification of AADMs. Red points are AADMₜₒₜₐₗ positive subjects, and black points are AADMₜₒₜₐₗ negative subjects.

FIG. 28 is a graph of ratios of concentrations of lysine to leucine obtained from the Quest analysis of subjects from the CAMP study.

FIG. 29 is a graph of ratios of concentrations of lysine to leucine obtained from the NeuroPoint diagnostic analysis of the 195 male subjects from the CAMP study.

FIG. 30 is a graph of ratios of concentrations of lysine to leucine obtained from the Quest diagnostic analysis of the 195 male subjects from the CAMP study.

FIG. 31 is a graph of ratios of concentrations of lysine to leucine obtained from the NeuroPoint diagnostic analysis of the 195 male subjects from the CAMP study.

FIG. 32 is a series of graphs showing of concentrations of taurine and homocitrulline and ratios of concentrations of taurine to homocitrulline obtained from the NeuroPoint diagnostic analysis of the 195 male subjects from the CAMP study.

FIG. 33 is a graph showing ratios of concentrations of alanine to tyrosine obtained from the NeuroPoint diagnostic analysis of the 195 male subjects from the CAMP study.

FIG. 34 is a graph showing concentrations of alanine obtained from the NeuroPoint diagnostic analysis of the 195 male subjects from the CAMP study.

FIG. 35 is a graph showing concentrations of tyrosine obtained from the NeuroPoint diagnostic analysis of the 195 male subjects from the CAMP study.

FIG. 36 is a graph showing ratios of concentrations of histidine to glutamine obtained from the NeuroPoint diagnostic analysis of the 195 male subjects from the CAMP study.

FIG. 37 is a graph showing concentrations of glutamine obtained from the NeuroPoint diagnostic analysis of the 195 male subjects from the CAMP study.

FIG. 38 is a graph showing concentrations of histidine obtained from the NeuroPoint diagnostic analysis of the 195 male subjects from the CAMP study.

FIG. 39 is a graph showing ratios of concentrations of taurine to homocitrulline obtained from the NeuroPoint diagnostic analysis of the 195 male subjects from the CAMP study.

FIG. 40 is a graph showing ratios of concentrations of taurine to homocitrulline obtained from the NeuroPoint diagnostic analysis of the 195 male subjects from the CAMP study.

FIG. 41 is a graph showing ratios of concentrations of alanine to tyrosine obtained from the NeuroPoint diagnostic analysis of the 195 male subjects from the CAMP study.

FIG. 42 is a graph showing ratios of concentrations of taurine to homocitrulline obtained from the NeuroPoint diagnostic analysis of the 195 male subjects from the CAMP study.

FIG. 43 is a graph showing the top 50 amino acid ratios according to the multivariate analysis.

FIG. 44 is a flow chart illustrating the analysis of data from MIND II study.

FIG. 45 is a graph showing hydrophilic interaction liquid chromatography electrospray ionization-negative (HILIC-ESIneg) and C8 electrospray ionization-negative (C8-ESIneg) data from subjects in the MIND II study.

FIG. 46 is a graph showing hydrophilic interaction liquid chromatography electrospray ionization-negative (HILIC-ESIneg) and C8 electrospray ionization-negative (C8-ESIneg) data from subjects in the ACHRI study.

FIG. 47 is a graph showing concentrations of CMPF obtained from analysis of subjects from the CAMP study.

FIG. 48 is a graph showing mass spectrometric peaks in a sample following NeuroPoint diagnostic analysis.

FIG. 49 is a graph showing ratios of concentrations of lysing to leucine obtained from the NeuroPoint diagnostic analysis of the 195 male subjects from the CAMP study.

FIG. 50 is a Venn diagram showing relationship of subjects having positive scores based on ratios of concentrations of glycine to isoleucine, glycine to leucine, and glycine to valine.

FIG. 51 is a graph showing ratios of concentrations of glycine to leucine obtained from the NeuroPoint diagnostic analysis of subjects from the CAMP study.

FIG. 52 is a graph showing ratios of concentrations of glycine to isoleucine obtained from the NeuroPoint diagnostic analysis of subjects from the CAMP study.

FIG. 53 is a graph showing ratios of concentrations of glycine to valine obtained from the NeuroPoint diagnostic analysis of subjects from the CAMP study.

FIG. 54 is a graph showing diagnostic value of ratios of concentrations of xanthine to uric acid obtained from diagnostic analysis of subjects from the CAMP study.

FIG. 55 is a graph showing diagnostic value of concentrations of uric acid obtained from diagnostic analysis of subjects from the CAMP study.

FIG. 56 is a graph showing diagnostic value of concentrations of xanthine obtained from diagnostic analysis of subjects from the CAMP study.

FIG. 57 is a Venn diagram showing the number of subjects having alterations in various metabolic pathways.

### DETAILED DESCRIPTION

The invention provides methods of diagnosing and treating autism spectrum disorders (ASD) by identification of altered metabolic pathways in such individuals. The invention recognizes that alterations in metabolic pathways can be identified from the ratios of concentrations of selected metabolites in a sample, such as a blood or plasma sample, from an individual. ASD, such as autism, Asperger syndrome, pervasive developmental disorder not otherwise specified (PDD-NOS), includes neurodevelopmental disorders that impair an individual's social and communication skills. Children with ASD are typically not diagnosed until 2-4 years of age, the age range at which their deficiencies in such skills become apparent. Although evidence suggests that certain environmental and genetic factors contribute to the development of ASD, no specific cause has yet been identified. Consequently, it is currently difficult to predict whether a given individual will develop as ASD prior to the onset of symptoms.

The invention is based on the insight that some ASD are associated with alterations in specific metabolic pathways, such as those involved in purine degradation, mitochondrial energy homeostasis, and neurotransmission and/or neurotransmitter synthesis. By analyzing ratios of concentrations of metabolites in such pathways, metabolic alterations can be detected at a very early age, well before the manifestation of behavioral symptoms. The early detection of an alteration in a metabolic pathway allows the dysregulation to be to treated or mitigated before it leads to neurodevelopmental abnormalities. Consequently, the effects of environmental and genetic factors that put individuals at risk of developing ASD can be mitigated, allowing normal or near-normal development in at-risk individuals.

### Autism Spectrum Disorders (ASD)

The autism spectrum includes a range of neurodevelopmental disorders associated with problems with social communication, social interaction, and restrict, repetitive patterns of behavior, interests, or activities. Disorders on the autism spectrum include autism, Asperger syndrome, pervasive developmental disorder not otherwise specified (PDD-NOS), and childhood disintegrative disorder.

The specific causes of ASD are not known. However, risk factors that contribute the development of ASD have been identified. For example, genetic factors play a role in the heritability of ASD, particular genetic conditions include variants of PTEN and SHANK3 and fragile X syndrome. Nonetheless, ASD cannot be attributed to specific mutations, and it is believed that a confluence of genetic variants is required for development of ASD. Advanced parental age is also associated with ASD. Other factors include gestational diabetes, bleeding after the first trimester of pregnancy, and the use of prescription medication such valproate during pregnancy.

ASD displays elevated rates of comorbidity with other disorders, such as seizure disorder, epilepsy, tuberous sclerosis, fragile X syndrome, Down syndrome, Prader-Willi and Angelman syndromes, Williams syndrome, learning disabilities, anxiety disorders, depression, and sensory processing disorder.

Metabolic Pathways Associated with ASD and Neurodevelopmental Disorders

The invention provides methods of identifying aberrant metabolic pathways associated with ASD and neurodevelopmental disorder by analysis of metabotypes. Ratios of concentrations of metabolites in a particular pathway may reveal alterations in activity of that pathways. Thus, any pathway that is dysregulated in an ASD or neurodevelopmental disorder may manifest a metabotype that can be used for methods of diagnosis and/or treatment.

One metabolic pathway that can be altered in ASD subjects is the purine degradation pathway. Flow of metabolites through the purine degradation pathway is shown below:

Xanthine oxidoreductase (XOR) is required for catabolism of purines. XOR catalyzes conversion of hypoxanthine to xanthine and xanthine to uric acid.

Changes in mitochondrial energy production pathways may also be associated with ASD subjects. Mitochondrial energy production pathways include the citric acid cycle (also called the tricarboxylic acid cycle or Krebs cycle) and oxidative phosphorylation (also called the electron transport chain). Key metabolites in these pathways include α-ketoglutarate, lactate, pyruvate, glutamate, and alanine, which are interconverted according to relation shown below:
Alterations in amine synthesis pathways are also associated with ASD subjects. Amine synthesis pathways are involved in synthesis of neurotransmitters, such as glutamate, aspartate, γ-aminobutyric acid (GABA), and glycine. Thus, defects in neurotransmitter synthesis or neurotransmission may be contribute to the clinical symptoms of ASD.

Dysregulation of other metabolic pathways may be associated with ASD subjects. For example, changes in metabolic pathways related to the gut microbiome or reactive oxidative species may also be identified in ASD subjects.

### Metabotypes

Various metabolites, such as amino acids, may be used in a ratio with another metabolite (for example, a “biological normalizer”). Amino acids represent a class of amine containing metabolites that include both proteinogenic and non-proteinogenic compounds. Each metabolite may be used in combination with one or more additional metabolites. For example, in some embodiments, one or more of the metabolites may be used in combination with one additional metabolite, two additional metabolites, three additional metabolites, four additional metabolites, five additional metabolites, six additional metabolites, seven additional metabolites, eight additional metabolites, nine additional metabolites, ten additional metabolites, or more.

For example, when the relationship of the metabolites of the ASD subjects was evaluated by correlation analysis and hierarchical clustering, two reproducible clusters of amine compounds were identified following permutation based analysis of the hierarchical clustering. Two independent clusters were identified through this analysis. Cluster one includes moderately positively correlated amine compounds such as serine, glycine, ornithine, 4-hydroxyproline, alanine, glutamine, homoscrine, and proline. Cluster two is a cluster with high positive correlation coefficients containing the branched chain amino acids (BCAAs) leucine, isoleucine, and valine. The ratios of metabolites in cluster 1 with the BCAAs of cluster 2 identified metabotypes related to ASD. Diagnostic thresholds applied to tcahese ratios define metabotypes associated with ASD uncovered potential metabotypes described by, for example, glutamine, glycine, homoserine, ornithine, and serine in ratios with BCAAs. Combinations of glutamine, glycine, and ornithine ratios with BCAAs identified a metabotype that is present in 15.4% of the CAMP ASD subjects and is detectable with a PPV of 93%.

Any naturally occurring amine containing compound or metabolite may be used as an analyte. For example and without limitation, potential amine-containing analytes include 4-hydroxyproline, alanine, arginine, asparagine, aspartic acid, beta-alanine, beta-aminoisobutyric acid, citrulline, ethanolamine, gamma-aminobutyric acid, glutamic acid, glutamine, glycine, histidine, homocitrulline, homoserine, hypoxanthine, inosine, isoleucine, kynurenine, leucine, lysine, methionine, ornithine, phenylalanine, proline, sarcosine, serine, serotonin, taurine, threonine, tryptophan, tyrosine, uric acid, valine, and xanthine. Metabolites that do not contain amines may also be used as analytes. For example and without limitation, potential analytes include alpha-ketoglutaric acid, lactic acid, pyruvic acid, and succinic acid.

Metabotypes include ratios of levels of specific metabolites. The ratios may be ratios of levels of individual metabolites, or the ratio may include the level of a class of metabolites, such as branched chain amino acids, e.g., leucine, isoleucine, and valine. Representative metabotypes are indicated in Table 1.

TABLE 1

| | |
|---|---|
| Metabotype 1 | Metabotype 1: An imbalance between the plasma |
| (GLN/ILE) | concentrations of Glutamine and Isoleucine was detected. |
| | This imbalance includes above average Glutamine and |
| | below average Isoleucine. |
| Metabotype 2 | Metabotype 2: An imbalance between the plasma |
| (GLN/LEU) | concentrations of Glutamine and Leucine was detected. |
| | This imbalance includes above average Glutamine and |
| | below average Leucine. |
| Metabotype 3 | Metabotype 3: An imbalance between the plasma |
| (GLY/ASN) | concentrations of Glycine and Asparagine was detected. |
| | This imbalance includes above average Glycine. |
| Metabotype 4 | Metabotype 4: An imbalance between the plasma |
| (GLY/GLU) | concentrations of Glycine and Glutamic Acid was |
| | detected. This imbalance includes above average |
| | Glycine and below average Glutamic Acid. |
| Metabotype 5 | Metabotype 5: An imbalance between the plasma |
| (GLY/ILE) | concentrations of Glycine and Isoleucine was detected. |
| | This imbalance includes above average Glycine and |
| | below average Isoleucine. |
| Metabotype 6 | Metabotype 6: An imbalance between the plasma |
| (GLY/LEU) | concentrations of Glycine and Leucine was detected. |
| | This imbalance includes above average Glycine and |
| | below average Leucine. |
| Metabotype 7 | Metabotype 7: An imbalance between the plasma |
| (GLY/VAL) | concentrations of Glycine and Valine was detected. |
| | This imbalance includes above average Glycine and |
| | below average Valine. |
| Metabotype 8 | Metabotype 8: An imbalance between the plasma |
| (ORN/PHE) | concentrations of Ornithine and Phenylalanine was |
| | detected. This imbalance includes above average |
| | Ornithine. |
| Metabotype 9 | Metabotype 9: An imbalance between the plasma |
| (ORN/VAL) | concentrations of Ornithine and Valine was detected. |
| | This imbalance includes above average Ornithine and |
| | below average Valine. |
| Metabotype 10 | Metabotype 10: An imbalance between the plasma |
| (GLN/BCAA) | concentrations of Glutamine and branched chain amino |
| | acids (BCAA) was detected. This imbalance includes |
| | above average Glutamine and below average BCAA. |
| Metabotype 11 | Metabotype 11: An imbalance between the plasma |
| (GLY/BCAA) | concentrations of Glycine and branched chain amino |
| | acids (BCAA) was detected. This imbalance includes |
| | above average Glycine and below average BCAA. |
| Metabotype 12 | Metabotype 12: An imbalance between the plasma |
| (ORN/BCAA) | concentrations of Ornithine and branched chain amino |
| | acids (BCAA) was detected. This imbalance includes |
| | above average Ornithine and below average BCAA. |
| Metabotype 13 | Metabotype 13: An imbalance between the plasma |
| (GLY/LYS) | concentrations of Glycine and Lysine was detected. |
| | This imbalance includes above average Glycine and |
| | below average Lysine. |
| Metabotype 14 | Metabotype 14: An imbalance between the plasma |
| (GLY/PHE) | concentrations of Glycine and Phenylalanine was |
| | detected. This imbalance includes above average |
| | Glycine and Phenylalanine below average. |
| Metabotype 15 | Metabotype 15: An imbalance between the plasma |
| (ORN/KY) | concentrations of Ornithine and Kynurenine was |
| | detected. This imbalance generally indicates plasma |
| | concentrations of Ornithine which are above average |
| | and kynurenine which is below average. |
| Metabotype 16 | Metabotype 16: An imbalance between the plasma |
| (ORN/LYS) | concentrations of Ornithine and Lysine was detected. |
| | This imbalance generally indicates plasma |
| | concentrations of Ornithine which are above average. |
| Metabotype 17 | Metabotype 17: An imbalance between the plasma |
| (SER/BCAA) | concentrations of Serine and the branched chain |
| | amino acids (BCAA) was detected. This imbalance |
| | includes above average Serine and below average BCAA. |
| Metabotype 18 | Metabotype 18: An imbalance between the plasma |
| (KYN2) | concentrations of Ethanolamine, Glutamic Acid, and |
| | Kynurenine. This imbalance includes elevated Glutamic |
| | Acid and decreased Kynurenine. |

Metabotypes are described in co-owned, co-pending International Application No. PCT/US2019/015673, the contents of which are incorporated herein by reference.

### Sample Collection

Samples may be obtained from any of a variety of mammalian subjects. In preferred embodiments, a sample is from a human subject.

A sample may be from an individual clinically diagnosed with ASD. ASD may be diagnosed by any of a variety of well-known clinical criteria. For example, diagnosis of autism spectrum disorder may be based on the DSM-IV criteria determined by an experienced neuropsychologist and/or the Autism Diagnostic Observation Schedule-Generic (ADOS-G) which provides observation of a child's communication, reciprocal social interaction, and stereotyped behavior including an algorithm with cutoffs for autism and autism spectrum disorders. A sample may be obtained from an individual previously diagnosed with autism spectrum disorder (ASD) and/or is undergoing treatment.

A sample may be obtained from an individual with a neurodevelopmental disorder.

A sample may be obtained from an individual determined to be developmentally delayed (DD), for example, demonstrating impairment in physical learning, language, and/or behavior

A sample may be obtained from an individual determined to be at some risk for ASD (for example by family history) with little or no current ASD symptoms. A sample may be a suitable reference or control sample from an individual not suffering from ASD with or without a family history of ASD. A sample may be obtained from a typically developing (TD) individual.

A sample may be obtained from a member of a subset of ASD subjects. The subset of ASD subjects may have a metabotype that is different from that of other ASD subjects, typically developing subjects, or both. The subset of ASD ratios may have a ratio of amine containing compounds that are different from ratios in other ASD subjects, in typically developing subjects, or in both.

A sample may be obtained from a phenotypic subpopulation of autism subjects, such as, for example, high functioning autism (HFA) or low functioning autism (LFA). A sample may be from an adult subject. A sample may be from a teenager. A sample may be from a child. A subject may be less than about 18 years of age, less than about 16 years of age, less than about 14 years of age, less than about 13 years of age, less than about 12 years of age, less than about 10 years of age, less than about 9 years of age, less than about 8 years of age, a child of less than about 7 years of age, a child of less than about 6 years of age, a child of less than about 5 years of age, a child of less than about 4 years of age, a child of less than about 3 years of age, a child of less than about 2 years of age, a child of less than about 18 months of age, a child of less than about 12 months of age, a child of less than about 9 months of age, a child of less than about 6 months of age, or a child of less than about 3 months of age, about 1 to about 6 years of age, about 1 to about 5 years of age, about 1 to about 4 years of age, about 1 to about 2 years of age, about 2 to about 6 years of age, about 2 to about 4 years of age, or about 4 to about 6 years of age.

In accordance with the methods disclosed herein, any type of biological sample that originates from anywhere within the body of a subject may be tested, including, but not limited to, blood (including, but no limited to serum or plasma), cerebrospinal fluid (CSF), pleural fluid, urine, stool, sweat, tears, breath condensate, saliva, vitreous humour, a tissue sample, amniotic fluid, a chorionic villus sampling, brain tissue, a biopsy of any solid tissue including tumor, adjacent normal, smooth and skeletal muscle, adipose tissue, liver, skin, hair, brain, kidney, pancreas, lung, colon, stomach, or the like may be used. A blood sample may include, for example, a whole blood sample, a blood serum sample, a blood plasma sample, or other blood components, such as, for example, a subfraction or an isolated cellular subpopulation of whole blood. In some aspects a sample may be a cellular membrane preparation. A sample may be from a live subject. In some applications, samples may be collected post mortem. A sample includes for example, cerebrospinal fluid, brain tissue, amniotic fluid, blood, serum, plasma, amniotic fluid, urine, breath condensate, sweat, saliva, tears, hair, cell membranes, and/or vitreous humour. In some aspects, a sample includes plasma.

When a blood sample is drawn from a subject, it can be processed in any of many known ways. The range of processing can be from little to none (such as, for example, frozen whole blood) or as complex as the isolation of a particular cell type. Common and routine procedures include the preparation of either serum or plasma from whole blood. All blood sample processing methods, including spotting of blood samples onto solid-phase supports, such as filter paper or other immobile materials, are contemplated by the present invention.

Samples may be collected repeatedly from a subject. For example, samples may be collected according to a schedule or at defined intervals, such as daily, weekly, biweekly, monthly, every two months, every three months, every four months, every six months, or annually.

Samples may be collected after a wash-out period, i.e., a period following a change in diet, medication, or other therapeutic program. The wash-out period allows the body to adapt to a new course of treatment and manifest an effect of the new treatment. The wash-out period may be about one day, about two days, about three days, about four days, about five days, about one week, about two weeks, about three weeks, about four weeks, about six weeks, about eight weeks, about twelve weeks, or more

### Analysis of Samples

With the preparation of samples for analysis, metabolites may be extracted from their biological source using any number of extraction/clean-up procedures that are typically used in quantitative analytical chemistry.

The metabolic markers and signatures described herein may be utilized in tests, assays, methods, kits for diagnosing, predicting, modulating, or monitoring ASD, including ongoing assessment, monitoring, susceptibility assessment, carrier testing and prenatal diagnosis.

Metabolic biomarkers may be identified by their unique molecular mass and consistency, thus the actual identity of the underlying compound that corresponds to the biomarker is not required for the practice of this invention. Biomarkers may be identified using, for example, Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis mass spectrometry, nuclear magnetic resonance spectrometry, tandem mass spectrometry (e.g., MS/MS. MS/MS/MS, ESI-MS/MS etc.), secondary ion mass spectrometry (SIMS), and/or ion mobility spectrometry (e.g. GC-IMS, IMS-MS, LC-IMS, LC-IMS-MS etc.).

Metabolites as set forth herein can be detected using any of the methods described herein. Metabolites, as set forth herein, can be detected using alternative spectrometry methods or other methods known in the art, in addition to any of those described herein.

In some aspects, the determination of a metabolite may be by a methodology other than a physical separation method, such as for example, a colorimetric, enzymatic, immunological methodology, and gene expression analysis, including, for example, real-time PCR, RT-PCR, Northern analysis, and in situ hybridization.

In some aspects, the quantification of one or more small molecule metabolites of a metabolic signature of autism may be assayed using a physical separation method, such as, for example, one or more methodologies selected from gas chromatography mass spectrometry (GCMS), C8 liquid chromatography coupled to electrospray ionization in positive ion polarity (C8ₚₒₛ). C8 liquid chromatography coupled to electrospray ionization in negative ion polarity (C8_{neg}), hydrophilic interaction liquid chromatography coupled to electrospray ionization in positive ion polarity (HILICₚₒₛ), and/or hydrophilic interaction liquid chromatography coupled to electrospray ionization in negative ion polarity (HILIC_{neg}).

With any of the methods described herein, any combination of one or more gas chromatography-mass spectrometry (GC-MS) methodologies and/or one or more liquid chromatography-high resolution mass spectrometry (LC-HRMS) methodologies may be used. In some aspects, a GC-MS method may be targeted. In some aspects, a LC-HRMS method may be untargeted. Subsequently, in some embodiments, tandem mass spectrometry (MS-MS) methods may be employed for the structural confirmation of metabolites. LC-HRMS methodologies may include C8 chromatography and/or Hydrophilic Interaction Liquid Chromatography (HILIC) chromatography. Either of C8 chromatography or HILIC chromatography may be coupled to electrospray ionization in both positive and negative ion polarities, resulting in multiple data acquisitions per sample.

In some aspects of the methods described herein, concentrations of one or more metabolites, including, but not limited to CMPF, may be determined using C18 (reverse phase) LC coupled with a triple quadrupole (QqQ) MS using electrospray ionization in the positive ion mode with analyte detection in the multiple reaction monitoring (MRM) mode. This may include a stable label internal standard and CMPF concentrations are measured distributed over a linear range of 0.05 to 100 μM.

In some embodiments, levels of amine containing compounds or metabolites are measured by mass spectrometry, optionally in combination with liquid chromatography. Molecules may be ionized for mass spectrometry by any method known in the art, such as ambient ionization, chemical ionization (CI), desorption electrospray ionization (DESI), electron impact (EI), electrospray ionization (ESI), fast-atom bombardment (FAB), field ionization, laser ionization (LIMS), matrix-assisted laser desorption ionization (MALDI), paper spray ionization, plasma and glow discharge, plasma-desorption ionization (PD), resonance ionization (RIMS), secondary ionization (SIMS), spark source, or thermal ionization (TIMS). Methods of mass spectrometry are known in the art and described in, for example, U.S. Pat. Nos. 8,895,918; 9,546,979; 9,761,426; Hoffman and Stroobant, Mass Spectrometry: Principles and Applications (2nd ed.). John Wiley and Sons (2001), ISBN 0-471-48566-7; Dass, Principles and practice of biological mass spectrometry, New York: John Wiley (2001) ISBN 0-471-33053-1; and Lee, ed., Mass Spectrometry Handbook, John Wiley and Sons, (2012) ISBN: 978-0-470-53673-5, the contents of each of which are incorporated herein by reference.

In certain embodiments, a sample can be directly ionized without the need for use of a separation system. In other embodiments, mass spectrometry is performed in conjunction with a method for resolving and identifying ionic species. Suitable methods include chromatography, capillary electrophoresis-mass spectrometry, and ion mobility. Chromatographic methods include gas chromatography, liquid chromatography (LC), high-pressure liquid chromatography (HPLC), and reversed-phase liquid chromatography (RPLC). In a preferred embodiment, liquid chromatography-mass spectrometry (LC-MS) is used. Methods of coupling chromatography and mass spectrometry are known in the art and described in, for example, Holcapek and Brydwell, eds. Handbook of Advanced Chromatography/Mass Spectrometry Techniques, Academic Press and AOCS Press (2017), ISBN 9780128117323; Pitt, Principles and Applications of Liquid Chromatography-Mass Spectrometry in Clinical Biochemistry, The Clinical Biochemist Reviews. 30(1): 19-34 (2017) ISSN 0159-8090; Niessen, Liquid Chromatography-Mass Spectrometry, Third Edition. Boca Raton: CRC Taylor & Francis. pp. 50-90. (2006) ISBN 9780824740825; Ohnesorge et al., Quantitation in capillary electrophoresis-mass spectrometry, Electrophoresis. 26 (21): 3973-87 (2005) doi: 10.1002/elps.200500398; Kolch et al., Capillary electrophoresis-mass spectrometry as a powerful tool in clinical diagnosis and biomarker discovery, Mass Spectrom Rev. 24 (6): 959-77. (2005) doi: 10.1002/mas.20051; Kanu et al., Ion mobility-mass spectrometry, Journal of Mass Spectrometry, 43 (1): 1-22 (2008) doi: 10.1002/jms.1383, the contents of which are incorporated herein by reference.

### Computer Systems

In some embodiments of the assays and/or methods described herein, the assay/method comprises or consists essentially of a system for doing one or more of the following steps: analyzing data, such as mass spectrometry data, to determine levels of amine containing compounds or metabolites in a sample; determining a ratio of two or more levels; and comparing a ratio from a sample to a reference ratio. If the comparison system, which may be a computer implemented system, indicates that the ratio in the sample is statistically higher or lower than the reference ratio, the subject from which the sample is collected may be identified as having or likely to develop an ASD.

The computer systems of the invention may include one or more of the following: (a) at least one memory containing at least one computer program adapted to control the operation of the computer system to implement a method that includes (i) a determination module configured to measure the levels of two or more amine containing compounds or metabolites in a test sample obtained from a subject; (ii) a storage module configured to store output data from the determination module; (iii) a computing module adapted to identify from the output data whether the ratio of levels in the test sample is statistically different from a reference ratio, and to provide a retrieved content; (iv) a display module for displaying for retrieved content (e.g., the ratio in the test sample and whether the test ratio is higher or lower than the reference ratio in a statistically significant manner); and (b) at least one processor for executing the computer program.

Embodiments may be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions may be carried out by the execution of various code portions stored on various media and executed at various times. Furthermore, it should be appreciated that the modules may perform other functions, thus the modules are not limited to having any particular functions or set of functions.

The computer-readable storage media may be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon may be loaded onto any computer resource to implement the aspects of the technology discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the technology described herein. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buchler, Bioinformatics Basics: Application in Biological Science and Medicine (CRC Press, London, 2000); and Ouclette and Bzevanis Bioinformatics: A Practical Guide for Analysis of Gene and Proteins (Wiley & Sons, Inc., 2nd ed., 2001), the contents of each of which are incorporated herein by reference.

The functional modules of certain embodiments may include at minimum a determination module, a storage module, a computing module, and a display module. The functional modules may be executed on one, or multiple, computers, or by using one, or multiple, computer networks. The determination module has computer executable instructions to provide e.g., levels of expression products in computer readable form.

The determination module may comprise any system for detecting a signal resulting from the ratio of levels of amine containing compounds or metabolites in a biological sample. In some embodiments, such systems may include an instrument, e.g., a plate reader for measuring absorbance. In some embodiments, such systems may include an instrument, e.g., the Cell Biosciences NANOPRO 1000™ System (Protein Simple; Santa Clara, CA) for quantitative measurement of proteins.

The information determined in the determination system may be read by the storage module. As used herein the “storage module” is intended to include any suitable computing or processing apparatus or other device configured or adapted for storing data or information. Examples of electronic apparatus suitable for use with the technology described herein include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, patient name, and numerical value of the ratio. Such information may be provided in digital form that may be transmitted and read electronically, e.g., via the Internet, on diskette, via USB (universal serial bus) or via any other suitable mode of communication. Those skilled in the art can readily adopt any of the presently known methods for recording information on known media to generate manufactures comprising expression level information.

In one embodiment of any of the systems described herein, the storage module stores the output data from the determination module. In additional embodiments, the storage module stores the reference information such as ratios of levels of amine containing compounds or metabolites samples obtained from typically developing subjects. In some embodiments, the storage module stores the information such as ratios of levels of amine containing compounds or metabolites in samples obtained from the same subject in earlier time points.

The computing module may use a variety of available software programs and formats for computing the ratios of levels of amine containing compounds or metabolites. Such algorithms are well established in the art. A skilled artisan is readily able to determine the appropriate algorithms based on the size and quality of the sample and type of data. The data analysis may be implemented in the computing module. In one embodiment, the computing module further comprises a comparison module, which compares the ratios of levels of amine containing compounds or metabolites in the test sample obtained from a subject as described herein with the reference ratio. For example, when the ratio in the test sample obtained from a subject is determined, a comparison module may compare or match the output data, e.g. with the reference ratio. In certain embodiments, the reference level has been pre-stored in the storage module. During the comparison or matching process, the comparison module may determine whether the ratio in the test sample obtained from a subject is higher or lower than the reference ratio to a statistically significant degree. In various embodiments, the comparison module may be configured using existing commercially-available or freely-available software for comparison purpose, and may be optimized for particular data comparisons that are conducted.

The computing and/or comparison module, or any other module, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware, as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment, users can directly access data (via Hypertext links for example) residing on Internet databases using a HTML interface provided by Web browsers and Web servers.

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module may be the relative ratio in the test sample obtained from a subject as compared to a reference ratio. In certain embodiments, the content displayed on the display module may indicate whether the ratio is found to be statistically significantly higher in the test sample obtained from a subject as compared to a reference ratio. In some embodiments, the content displayed on the display module may show the ratios from the subject measured at multiple time points, e.g., in the form of a graph. In some embodiments, the content displayed on the display module may indicate whether the subject has an ASD. In certain embodiments, the content displayed on the display module may indicate whether the subject is in need of a treatment for an ASD.

In one embodiment, the content based on the computing and/or comparison result is displayed on a computer monitor. In one embodiment, the content based on the computing and/or comparison result is displayed through printable media. The display module may be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, California, or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules may be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the technology relating to determining the ratios of level of amine containing compounds or metabolites, and therefore are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Data Analysis

In some aspects, the minimum percentage sensitivity required for the determination of a hypothetical diagnostic includes about 3%, about 4%, about 5%, about 7%, about 8%, about 9%, about 10%, about 11%, about 12%, about 13%, about 14%, about 15%, about 16%, about 17%, about 18%, about 19%, or about 20%, rather than about 6%; the ratio diagnostics perform with greater than at least about 95% specificity, at least about 96% specificity, at least about 97% specificity, at least about 98% specificity, or at least about 99% specificity; and/or the ratio diagnostics perform with at least about 75% specificity, at least about 80% specificity, at least about 85% specificity, at least about 86% specificity, at least about 87% specificity, at least about 88% specificity, or at least about 89% specificity, rather than greater than about 90% specificity.

Data collected during analysis may be quantified for one or more than one metabolite. Quantifying data may be obtained by measuring the levels or intensities of specific metabolites present in a sample. The quantifying data may be compared to corresponding data from one or more than one reference sample. For example, a reference sample may be a sample from a control individual, i.e., a person not suffering from ASD with or without a family history of ASD (also referred to herein as a “typically developing individual” (TD), or “normal” counterpart). A reference sample may also be a sample obtained from a patient clinically diagnosed with ASD. A reference sample may be a sample from a member of a subset of ASD subjects. Subjects in the subset may have a ratio of concentrations of two or more amine containing compounds that is different from the ratio of concentrations of the two or more amine containing compounds in other ASD subjects, in typically developing subjects, or in both. For example, the subset may include ASD subjects of a particular metabotype. As would be understood by a person of skill in the art, more than one reference sample may be used for comparison to the quantifying data.

Sensitivity and specificity are statistical measures of the performance of a binary classification test. Sensitivity measures the proportion of actual positives which are correctly identified as such (e.g. the percentage of sick people who are correctly identified as having the condition). Specificity measures the proportion of negatives which are correctly identified (e.g. the percentage of healthy people who are correctly identified as not having the condition). These two measures are closely related to the concepts of type I and type II errors. A theoretical, optimal prediction can achieve 100% sensitivity (i.e. predict all people from the sick group as sick) and 100% specificity (i.e. not predict anyone from the healthy group as sick). A specificity of 100% means that the test recognizes all actual negatives—for example, in a test for a certain disease, all disease free people will be recognized as disease free. A sensitivity of 100% means that the test recognizes all actual positives—for example, all sick people are recognized as being ill. Thus, in contrast to a high specificity test, negative results in a high sensitivity test are used to rule out the disease. A positive result in a high specificity test can confirm the presence of disease. However, from a theoretical point of view, a 100%-specific test standard can also be ascribed to a ‘bogus’ test kit whereby the test simply always indicates negative. Therefore, the specificity alone does not tell us how well the test recognizes positive cases. Knowledge of sensitivity is also required. For any test, there is usually a trade-off between the measures. For example, in a diagnostic assay in which one is testing for people who have a certain condition, the assay may be set to overlook a certain percentage of sick people who are correctly identified as having the condition (low specificity), in order to reduce the risk of missing the percentage of typically developing people who are correctly identified as not having the condition (high sensitivity). Eliminating the systematic error improves accuracy but does not change precision. This trade off can be represented graphically using a receiver operating characteristic (ROC) curve.

The accuracy of a measurement system is the degree of closeness of measurements of a quantity to its actual (true) value. The “precision” of a measurement system, also called reproducibility or repeatability, is the degree to which repeated measurements under unchanged conditions show the same results. Although the two words can be synonymous in colloquial use, they are deliberately contrasted in the context of the scientific method. A measurement system can be accurate but not precise, precise but not accurate, neither, or both. For example, if an experiment contains a systematic error, then increasing the sample size generally increases precision but does not improve accuracy.

Predictability (also called banality) is the degree to which a correct prediction or forecast of a system's state can be made either qualitatively or quantitatively. Perfect predictability implies strict determinism, but lack of predictability does not necessarily imply lack of determinism. Limitations on predictability could be caused by factors such as a lack of information or excessive complexity.

In some embodiments, the invention discloses a method for diagnosing autism with at least about 80% accuracy, at least about 81% accuracy, at least about 82% accuracy, at least about 83% accuracy, at least about 84% accuracy, at least about 85% accuracy, at least about 86% accuracy, at least about 87% accuracy, at least about 88% accuracy, at least about 89% accuracy, at least about 90% accuracy, at least about 91% accuracy, at least about 92% accuracy, at least about 93% accuracy, at least about 94% accuracy, at least about 95% accuracy, at least about 96% accuracy, at least about 97% accuracy, at least about 98% accuracy, or at least about 99% accuracy.

In some embodiments, the invention discloses a method for diagnosing autism with at least about 80% sensitivity, at least about 81% sensitivity, at least about 82% sensitivity, at least about 83% sensitivity, at least about 84% sensitivity, at least about 85% sensitivity, at least about 86% sensitivity, at least about 87% sensitivity, at least about 88% sensitivity, at least about 89% sensitivity, at least about 90% sensitivity, at least about 91% sensitivity, at least about 92% sensitivity, at least about 93% sensitivity, at least about 94% sensitivity, at least about 95% sensitivity, at least about 96% sensitivity, at least about 97% sensitivity, at least about 98% sensitivity, or at least about 99% sensitivity.

In some embodiments, the invention discloses a method for diagnosing autism with at least about 75% specificity, at least about 80% specificity, at least about 81% specificity, at least about 82% specificity, at least about 83% specificity, at least about 84% specificity, at least about 85% specificity, at least about 86% specificity, at least about 87% specificity, at least about 88% specificity, at least about 89% specificity, at least about 90% specificity, at least about 91% specificity, at least about 92% specificity, at least about 93% specificity, at least about 94% specificity, at least about 95% specificity, at least about 96% specificity, at least about 97% specificity, at least about 98% specificity, or at least about 99% specificity.

Data analysis may include comparing a ratio of levels of amine containing compounds or other metabolites in a sample from a test subject to a corresponding ratio from one or more reference subjects. The latter number may be referred to as a reference ratio. The reference ratio may be defined based on clinical trials that determine the ratio of levels of amine containing compounds or metabolites that that optimally defines a cut-off point above which the likelihood of occurrence of an ASD is high and below which the likelihood of occurrence of an ASD is low.

The reference ratio may be defined by a statistic describing the distribution of ratios in typically developing subjects. The reference ratio may be above the highest observed ratio in a sample from a typically developing subject or a population of typically developing subjects, or the reference ratio may be below the lowest observed ratio in a sample from a typically developing subject or a population of typically developing subjects. Any ratio above or below the reference ratio may be deemed to be significantly different from the average ratio in a sample from a typically developing subject or a population of typically developing subjects. The reference ratio may be greater than 95% of the ratios observed in samples from a typically developing subject or a population of typically developing subjects, or it may be above the lower limit of the highest decile, quartile or tertile of the ratios observed in samples from a typically developing subject or a population of typically developing subjects. Alternatively, the reference ratio may be less than 95% of the ratios observed in samples from a typically developing subject or a population of typically developing subjects, or it may be lower than the upper limit of the lowest decile, quartile or tertile of the ratios observed in samples from a typically developing subject or a population of typically developing subjects.

The reference ratio may be defined by a statistic describing the distribution of ratios in a subset of ASD subjects. The reference ratio may be above the highest observed ratio in a sample from a subset of ASD subjects, or the reference ratio may be below the lowest observed ratio in a sample from a subset of ASD subjects. Any ratio above or below the reference ratio may be deemed to be significantly different from the average ratio in a sample from a member of a subset of ASD subjects. The reference ratio may be greater than 95% of the ratios observed in samples from a subset of ASD subjects, or it may be above the lower limit of the highest decile, quartile or tertile of the ratios observed in samples from a subset of ASD subjects. Alternatively, the reference ratio may be less than 95% of the ratios observed in samples from a subset of ASD subjects, or it may be lower than the upper limit of the lowest decile, quartile or tertile of the ratios observed in samples from a subset of ASD subjects.

The reference ratio may be at least one standard deviation, at least two standard deviations, or at least three standard deviations above or below the average ratio in a sample from a typically developing subject or a population of typically developing subjects. Any ratio above or below the reference ratio may be deemed to be significantly different from the average ratio in a sample from a typically developing subject or a population of typically developing subjects.

The reference ratio may be at least one standard deviation, at least two standard deviations, or at least three standard deviations above or below the average ratio in a sample from a subset of ASD subjects. Any ratio above or below the reference ratio may be deemed to be significantly different from the average ratio in a sample from a subset of subjects.

The reference ratio may be a ratio in a sample of the same subject measured at an earlier time point. The reference level may be a ratio in a sample obtained from the same subject before commencement of a therapeutic program, such as an altered diet and/or course of medication. The reference ratio may be from a sample obtained 1 hour, 2 hours, 4 hours, 6 hours, 8 hours, 12 hours, 24 hours, 36 hours, 2 days, 3 days, 4 days, 5 days, 6 days, 7 days or more before commencing the course of therapy.

The reference ratio may be at least one standard deviation, at least two standard deviations, or at least three standard deviations above or below a ratio in a sample obtained from the same subject at an earlier time point. Any ratio above or below the reference ratio may be deemed to be significantly different from the ratio in the earlier sample.

In some embodiments, the ratio of level of amine containing compounds or metabolites measured in a sample from a subject identified as having an ASD may be at least 5%, at least 10%, at least 20%, at least 30%, at least 40%, at least 50%, at least 60%, at least 70%, at least 80%, at least 90%, at least 100%, at least 150%, at least 200%, or at least 300% higher or lower than the reference ratio.

The reference ratio may be adjusted to account for variables such as sample type, gender, age, weight, and ethnicity. Thus, reference ratios accounting for these and other variables may provide added accuracy for the methods described herein.

### Guidance for Treatment

With any of the methods described herein, the method may further include providing guidance for individualized treatment to the one or more individuals identified as belonging to an autism subpopulation. In some aspects, individualized treatment includes modified diet, dietary supplements, probiotic therapy, medical grade food, and/or pharmacological therapy. In some aspects, the level of the one or more metabolites indicative of ASD and/or an ASD subset returns to TD levels after initiation of treatment. The methods may include providing the treatment to the subject.

The dietary modification may include supplementation with a source of amine containing compounds. For example, the dietary modification may be a protein-rich diet. The dietary modification may include supplementation with specific amine containing compounds or amino acids. For example, the dietary modification may include supplementation with one or more branched chain amino acids, such as isoleucine, leucine, or valine. Providing additional branched chain amino acids in the diet may alter ratios of levels of amino acids that are associated with ASD and therefore may prevent development of an ASD or mitigate the severity of an ASD.

The dietary modification may include a source of amine containing compounds or amino acids that is substantially free of phenylalanine. For example, patients with phenylketonuria are unable to metabolize phenylalanine, which can lead to intellectual disability, seizures, behavioral problems, and mental disorders. The milk peptide casein glycomacropeptide (CGMP) is naturally free of pure phenylalanine. Therefore, dietary supplementation with glycomacropeptide provides other amine containing compounds or amino acids s but not phenylalanine. The use of glycomacropeptide for preparation of medical foods is known in the art and described in, for example, U.S. Pat. Nos. 5,968,586; 6,168,823; and 8,604,168, the contents of each of which are incorporated herein by reference.

The guidance may provide recommendations for dietary modification. For example, the guidance may include specific formulations, such as beverages, powder, mixes, protein shakes, and the like, to provide one or more amine containing compounds or amino acids. The guidance may include a recommended quantity of one or more therapeutic dietary supplements.

The guidance may provide recommendations for specific medications to treat the ASD or one or more symptoms associated with an ASD.

The guidance may include a recommended schedule for a course of treatment, such as a dietary modification or medication regimen. For example, the guidance may recommend taking a supplement or medication once per day, twice per day, three times per day, or more. The guidance may include a recommended duration for a course of treatment. The duration may be one week, two weeks, three weeks, one month, two months, three months, four months, six months, one year, two years, three years, four year, five years, or more.

The guidance may include a recommendation that the subject be evaluated by a specialist. For example, the guidance may include a recommendation that the subject consult with a neurodevelopment specialist or nutritionist.

The guidance may include metrics or criteria for evaluating developmental progress of the subject. For example and without limitation, the metrics may include measures of growth, such as height and weight, hyperactivity, irritability, communication skills, socialization, or academic performance of the subject.

The guidance may be communicated in any suitable manner. For example, the guidance may be provided in a written report. The guidance may be shown on a display device, such as a computer monitor, telephone, portable electronic device, or the like.

The report may contain additional information about the subject, such as age, sex, weight, height, genetic data, genomic data, and dietary preferences.

The report may indicate that the test subject has or is at risk of developing a neurodevelopmental disorder if the test ratio is imbalanced compared to the reference ratio. The report may indicate a likelihood or probability that the test subject will develop a neurodevelopmental disorder. The report may indicate a likelihood or probability that the test subject will develop a neurodevelopmental disorder if the test subject goes untreated. The report may indicate a likelihood or probability that the test subject will develop a neurodevelopmental disorder if the test subject undergoes a particular course of treatment, such as a dietary modification.

### Kits

The present invention includes kits for identifying and/or measuring one or more metabolites associated with a subset of ASD. In some aspects, the kit may be for the determination of a metabolite by a physical separation method. In some aspects, the kit may be for the determination of a metabolite by a methodology other than a physical separation method, such as for example, a colorimetric, enzymatic, immunological methodology. In some aspects, an assay kit may also include one or more appropriate negative controls and/or positive controls. Kits of the present invention may include other reagents such as buffers and solutions needed to practice the invention are also included. Optionally associated with such container(s) can be a notice or printed instructions. As used herein, the phrase “packaging material” refers to one or more physical structures used to house the contents of the kit. The packaging material is constructed by well-known methods, preferably to provide a sterile, contaminant-free environment. As used herein, the term “package” refers to a solid matrix or material such as glass, plastic, paper, foil, and the like. Kits of the present invention may also include instructions for use. Instructions for use typically include a tangible expression describing the reagent concentration or at least one assay method parameter, such as the relative amounts of reagent and sample to be admixed, maintenance time periods for reagent/sample admixtures, temperature, buffer conditions, and the like. In some aspects, a kit may be a packaged combination including the basic elements of a first container including, in solid form, a specific set of one or more purified metabolites, as described herein, and a second container including a physiologically suitable buffer for resuspending or dissolving the specific subset of purified metabolites. Such a kit may be used by a medical specialist to determine whether or not a subject is at risk for ASD. Appropriate therapeutic intervention may be prescribed or initiated upon the determination of a risk of ASD. One or more of the metabolites described herein may be present in a kit.

### EXAMPLES

### Example 1: Introduction

Autism Spectrum Disorder (ASD) is characterized by core symptoms of altered social communication and repetitive behaviors or circumscribed interests (1) and has a prevalence of 1:59 children in the United States. Affected individuals vary enormously in the severity of their autistic characteristics as well as in the occurrence of many co-morbid conditions. Co-morbid conditions include intellectual disability which affects at least 40% of individuals with autism (2-4); anxiety in approximately 50% (5); epilepsy in approximately 25% (3, 4); and gastrointestinal disorders in approximately 25% of autistic individuals (6). Twin studies (7, 8) have indicated that genetic factors play a prominent role in the etiology of ASD although the genetics of autism appears to be extremely complex. There has been enormous progress in establishing the genetic architecture of ASD and there are at least 100 genes known to confer risk of ASD (9, 10). There is also increasingly strong evidence that environmental factors, alone or in conjunction with genotype, can contribute to the risk for ASD (11). These findings have led to a widespread consensus that there are different biological forms of ASD that may necessitate different diagnostic, preventative, and treatment strategies.

ASD is currently diagnosed based on behavioral characteristics exhibited by an affected child (12). While specialist clinicians are able to confidently diagnose children as young as 24 months (13), the average age of diagnosis in the United States is over 4 years (2, 14). Families often experience long waits to receive a definitive diagnosis due to the paucity of trained clinicians able to perform diagnostic assessment. Early diagnosis is important because intensive behavioral therapies are not only effective in reducing disability in many children with autism (15-17), but the benefit of early intervention is greater the earlier the intervention is started.

Unfortunately, there is currently no reliable biomarker that can be used to identify children at risk for ASD (18). Because of the genetic complexity of ASD, there is currently no clinically meaningful genotyping carried out to detect ASD. There have been recent intriguing neuroimaging studies indicating that alterations of brain function or structure as early as 6 months may be valuable indicators of a higher risk for autism (19, 20). However, it is unlikely that comprehensive structural and functional magnetic resonance imaging is a practical approach to detecting ASD in young children. Other, more cost effective and widely applicable biomarker strategies must be discovered.

We previously demonstrated that a metabolomics approach for the detection of autism risk holds substantial promise (21). In our preliminary study, we identified a subset of 179 features that classified ASD and TYP children with 81% accuracy. Metabolism-based analysis has the merit of being sensitive to interactions between the genome, gut microbiome, diet, and environmental factors that contribute to the unique metabolic signature of an individual (22). Metabolic testing can provide important biomarkers toward identifying the perturbations of biological processes underlying an individual's ASD. Past studies have been underpowered to identify metabolic perturbations that lead to actionable metabolic subtypes (23).

To test for metabolic imbalances that can reveal subtypes of ASD subjects, we conducted the Children's Autism Metabolome Project (CAMP, ClinicalTrials.gov Identifier NCT02548442). CAMP recruited 1,100 young children (18 to 48 months) with ASD, intellectual disability or typical development. Research reliable clinicians confirmed the child's diagnosis and blood samples were collected under protocols designed specifically for metabolomics analyses. The CAMP study is the largest metabolomics study of ASD to date.

The current study was motivated by observations of AA dysregulation in West et al. (21) and in preliminary analysis of the CAMP samples. The relevance of AA dysregulation to ASD is reinforced by Novarino (24) who demonstrated loss of function mutations in the gene BCKDK (Branched Chain Ketoacid Dehydrogenase Kinase) resulting in reductions of BCKDK messenger RNA and protein, Ela phosphorylation, and plasma branched-chain AAs in consanguineous families with autism, epilepsy, and intellectual disability. Follow on studies by Tarlungeanu (25) demonstrated that altered transport of BCAAs across the blood brain barrier led to dysregulation of AA levels and neurological impairments. We sought to determine whether dysregulation of AAs was a more pervasive phenomenon in individuals with ASD. Thus, we set out to identify metabotypes indicating the dysregulation of AAs in individuals with ASD and to determine whether these metabotypes might be diagnostic of subsets of individuals. A metabotype is a subpopulation defined by a common metabolic signature that can be differentiated from other members of the study population (26). Metabotypes of ASD can be useful in stratifying the broad autism spectrum into more biochemically homogeneous and clinically significant subtypes. Stratification of ASD based on distinct metabolism can inform pharmacological and dietary interventions that prevent or ameliorate clinical symptoms within a metabotype.

### Example 2: Methods and Materials

### CAMP Participants

The CAMP study recruited children, ages 18 to 48 months, from 8 centers across the United States as shown in Table 2.

TABLE 2

| Clinical Site | Location |
|---|---|
| MIND Institute, University of California at Davis | Sacramento, CA |
| Nationwide Children's Hospital | Columbus, OH |
| The Melmed Center | Scottsdale, AZ |
| Vanderbilt University Medical Center | Nashville, TN |
| The University of Arkansas for Medical Sciences | Little Rock, AR |
| Cincinnati Children's Hospital | Cincinnati, OH |
| Children's Hospital of Philadelphia | Philadelphia, PA |
| Massachusetts General Hospital | Lexington, MA |

Informed consent of a parent or legal guardian was obtained for each participant. The study protocol was approved and monitored by local IRBs at each of the sites. Enrollment was limited to one child per household to minimize genetic or family environmental effects. Children participating in other clinical studies could not have used any investigational agent within 30 days of participation. Children were excluded from the study if they were previously diagnosed with a genetic condition such as fragile X syndrome, Rett syndrome, Down syndrome, tuberous sclerosis, or inborn errors of metabolism. Subjects that had fetal alcohol syndrome, or other serious neurological, metabolic, psychiatric, cardiovascular, or endocrine system disorders were also excluded. In addition, children exhibiting signs of illness within 2 weeks of enrollment, including vomiting, diarrhea, fever, cough, or ear infection were rescheduled. Each participant underwent physical, neurological and behavioral examinations. Metadata was obtained about the children's birth, developmental, medical and immunization histories, dietary supplements and medications. Parents' medical histories were also obtained.

The Autism Diagnostic Observations Schedule-Second Version (ADOS-2) was performed by research reliable clinicians to confirm ASD diagnoses. The Mullen Scales of Early Learning (MSEL) was administered to establish a developmental quotient (DQ) for all children in the study. A prior ADOS-2 or MSEL was accepted if performed within 90 days of enrollment by qualified personnel. Children without ASD receiving a clinical diagnosis of developmental delay were not included in the current study. Children entering the study as TYP were not routinely administered the ADOS-2. The Social Communications Questionnaire (SCQ) was administered for a subset of 65 TYP children as a screen for ASD. Of these, 9 were referred for subsequent ADOS-2 evaluation. Four received a diagnosis of ASD (and were included in this study) and 5 received a diagnosis of TYP.

### Training and Test Sets

A training set was used to identify metabotypes associated with ASD and a test set was used to evaluate the reproducibility of the metabotypes. The sample size of the training set was designed to detect metabotypes with a sensitivity (metabotype prevalence)>3% and specificity >85% with a power of 0.90. The power analysis and minimum sample size requirements for metabotype identification are shown in Table 3.

TABLE 3

| Type I and II Error | | | Sensitivity Sample Size Requirements | | | |
|---|---|---|---|---|---|---|
| Alpha | Tails | Minimum | Expected | Lower | Lower | Subject |
| | | Power | Sensitivity | Confidence Limit | Sensitivity | Number |
| | | | | Sensitivity | Limit | ASD |
| 0.05 | 1 | 0.9 | 0.08 | 0.05 | 0.03 | 252 |

| Specificity Sample Size Requirements | | | |
|---|---|---|---|
| Expected | Lower | Lower | Subject |
| Specificity | Confidence Limit | Specificity | Number |
| | Specificity | Limit | TYP |
| 0.95 | 0.1 | 0.85 | 87 |

Sample sizes were determined using equation A1 in Autism, Developmental Disabilities Monitoring Network Surveillance Year Principal I, Centers for Disease C, Prevention (2012): Prevalence of autism spectrum disorders-Autism and Developmental Disabilities Monitoring Network, 14 sites, United States, 2008. MMWR Surveill Summ. 61: 1-19, the contents of which are incorporated herein by reference
Abbreviations: ASD, autism spectrum disorder; TYP, typically developing.

The training set (N=338, ASD=253, TYP-85) was established and analyzed, then as recruitment continued, the test set (N=342, ASD=263, TYP=79) was established when sufficient subjects were available to match the training set demographics. Subject composition of training and tests are shown in Table 4.

TABLE 4

| Metric | Training Set | Test Set | Combined Sets |
|---|---|---|---|
| ASD Children | 253 | 263 | 516 |
| TYP Children | 85 | 79 | 164 |
| ASD Prevalence (%) | 74.9 | 76.9 | 75.9 |
| ASD % Male | 77.9 | 79.5 | 78.7 |
| TYP % Male | 64.7 | 59.5 | 62.2 |
| ASD Age (Months) | 35.9 +/− 7.5 | 34.5 +/− 7.9 | 35.2 +/− 7.8 |
| TYP Age (Months) | 32.6 +/− 8.5 | 31.3 +/− 8.8 | 32 +/− 8.7 |
| Age (range) | 18 to 48 | 18 to 48 | 18 to 48 |
| DQ ASD | 62 +/− 17.8 | 63.5 +/− 17.7 | 62.8 +/− 17.8 |
| DQ TYP | 98.5 +/− 14.7 | 101.8 +/− 18.2 | 100.1 +/− 16.5 |

Values are means +/− standard deviation.
Abbreviations: TYP, typically developing; ASD, autism spectrum disorder; DQ, developmental quotient.

### Phlebotomy Procedures

Blood was collected from subjects after at least a 12 hour fast by venipuncture into 6 ml sodium heparin tubes on wet ice. A minimum of a 2 ml blood draw was required for sample inclusion in the computational analyses. The plasma was obtained by centrifugation (1200×G for 10 minutes) and frozen to −70° C. within 1 hour.

### Triple Quadrupole LC-MS/MS Method for Quantitative Analysis of Biological Amines

The Waters AccQTag™ Ultra kit (Waters Corporation, Milford, MA), which employs derivatization of amine moieties with 6-aminoquinolyl-N-hydroxysuccinimidyl carbamate was employed for all samples prior to multiple reaction monitoring (MRM) on a liquid chromatography (LC) mass spectrometry (MS) system consisting of an Agilent 1290 ultra-high performance liquid chromatograph (UHPLC) coupled to an Agilent G6490 Triple Quadrupole Mass Spectrometer (Agilent Technologies Santa Clara, CA). Endogenous metabolites chemical reference standards, ions used for quantitation, and the stable isotope labeled (SIL) internal standard used for normalization are shown in Table 5.

TABLE 5

| | Stable Isotope Labeled | | | | |
|---|---|---|---|---|---|
| | Standard Used for | Precursor | Product | Ret Time | |
| Compound Name | Quantification | Ion | Ion | (min) | Product Information |
| 4-hydroxyproline | Arginine-13C6, 15N4 | 302.0 | 171.1 | 1.70 | A9906. Sigma-Aldrich, St. Louis MO |
| Alanine | Alanine-13C6, 15N | 260.1 | 116.1 | 3.65 | A9906, 05129. Sigma-Aldrich, St. |
| | | | | | Louis MO |
| Arginine | Arginine-13C6, 15N4 | 345.1 | 171.1 | 1.98 | A9906. Sigma-Aldrich, St. Louis MO |
| Asparagine | Asparagine-D3 | 303.1 | 171.1 | 1.98 | A0884. Sigma-Aldrich, St. Louis MO |
| Aspartic Acid | Aspartic Acid-13C4, 15N | 304.1 | 171.1 | 2.80 | A9906. Sigma-Aldrich, St. Louis MO |
| Beta-Alanine | Alanine-13C6, 15N | 260.1 | 116.1 | 3.20 | A9906. Sigma-Aldrich, St. Louis MO |
| Beta-Aminoisobutyric acid | Glycine-13C2, 15N | 274.1 | 171.1 | 3.99 | A9906. Sigma-Aldrich, St. Louis MO |
| Citrulline | Citrulline-D4 | 346.2 | 171.1 | 2.90 | A9906. Sigma-Aldrich, St. Louis MO |
| Ethanolamine | Ethanolamine -D7 | 232.1 | 171.1 | 2.51 | A9906. Sigma-Aldrich, St. Louis MO |
| Gamma-Aminobutyric Acid | Alanine-13C6, 15N | 274.1 | 171.1 | 3.68 | A9906. Sigma-Aldrich, St. Louis MO |
| Glutamic Acid | Glutamic Acid-13C5, 15N | 318.1 | 171.1 | 3.05 | A9906. Sigma-Aldrich, St. Louis MO |
| Glutamine | Glutamine-13C5 | 317.1 | 171.1 | 2.33 | G8540. Sigma-Aldrich, St. Louis MO |
| Glycine | Glycine-13C2, 15N | 246.1 | 171.1 | 2.61 | A9906, 76524. Sigma-Aldrich, St. |
| | | | | | Louis MO |
| Histidine | Histidine-13C6, 15N3 | 326.1 | 156.1 | 0.99 | A9906. Sigma-Aldrich, St. Louis MO |
| Homocitrulline | Citrulline-D4 | 360.2 | 171.1 | 3.65 | H590900. Toronto Research |
| | | | | | Chemicals, North York ON Canada |
| Homoserine | Serine-13C3, 15N | 290.1 | 171.1 | 2.52 | H6515. Sigma-Aldrich, St. Louis MO |
| Isoleucine | Isoleucine-13C6, 15N | 302.1 | 171.1 | 5.49 | A9906. Sigma-Aldrich, St. Louis MO |
| Kynurenine | Kynurenine-D6 | 379.2 | 171.1 | 5.55 | K8625. Sigma-Aldrich, St. Louis MO |
| Leucine | Leucine-13C6, 15N | 302.1 | 171.1 | 5.56 | A9906. Sigma-Aldrich, St. Louis MO |
| Lysine | Lysine-13C6, 15N | 244.2 | 171.1 | 4.31 | A9906. Sigma-Aldrich, St. Louis MO |
| Methionine | Methionine-13C5, 15N | 320.1 | 171.1 | 4.83 | A9906. Sigma-Aldrich, St. Louis MO |
| Ornithine | Ornithine-D7 | 303.1 | 171.1 | 4.08 | A9906. Sigma-Aldrich, St. Louis MO |
| Phenylalanine | Phenylalanine-13C9, 15N | 336.1 | 171.1 | 5.70 | A9906. Sigma-Aldrich, St. Louis MO |
| Proline | Proline-13C5, 15N | 286.1 | 171.1 | 3.98 | A9906. Sigma-Aldrich, St. Louis MO |
| Sarcosine | Sarcosine-D3 | 260.1 | 116.1 | 2.95 | A9906. Sigma-Aldrich, St. Louis MO |
| Serine | Serine-13C3, 15N | 276.1 | 171.1 | 2.34 | A9906. Sigma-Aldrich, St. Louis MO |
| Serotonin | Serotonin-D4 | 347.2 | 171.1 | 4.95 | 14927. Sigma-Aldrich, St. Louis MO |
| Taurine | Taurine-D4 | 296.1 | 171.1 | 2.31 | A9906. Sigma-Aldrich, St. Louis MO |
| Threonine | Threonine-13C4, 15N | 290.1 | 171.1 | 3.26 | A9906. Sigma-Aldrich, St. Louis MO |
| Tryptophan | Tryptophan-D3 | 375.1 | 171.1 | 5.77 | A9906. Sigma-Aldrich, St. Louis MO |
| Tyrosine | Tyrosine-13C9, 15N | 352.1 | 171.1 | 4.70 | A9906. Sigma-Aldrich, St. Louis MO |
| Valine | Valine-13C5, 15N | 288.1 | 171.1 | 4.88 | A9906. Sigma-Aldrich, St. Louis MO |

Stable isotope labeled (SIL) chemical reference standards and ions used for quantification are shown in Table 6.

TABLE 6

| | Precursor | Product | Ret Time | |
|---|---|---|---|---|
| Compound Name | Ion | Ion | (min) | Product Information |
| Alanine-13C6, 15N | 264.1 | 116.1 | 3.65 | MSK-A2-S. Cambridge Isotope Laboratories, Inc., Andover MA |
| Arginine-13C6, 15N4 | 355.1 | 171.1 | 1.98 | MSK-A2-S. Cambridge Isotope Laboratories, Inc., Andover MA |
| Asparagine-D3 | 306.1 | 171.1 | 1.98 | A790007. Toronto Research Chemicals, North York ON Canada |
| Aspartic Acid-13C4, 15N | 309.1 | 171.1 | 2.80 | MSK-A2-S. Cambridge Isotope Laboratories, Inc., Andover MA |
| Citrulline-D4 | 350.2 | 171.1 | 2.89 | DLM-6039-0. Cambridge Isotope Laboratories, Inc., Andover MA |
| Ethanolamine -D7 | 239.1 | 171.1 | 2.51 | D-6816. CDN Isotopes, Pointe-Claire, QC Canada |
| Glutamic Acid-13C5, 15N | 324.1 | 171.1 | 3.05 | MSK-A2-S. Cambridge Isotope Laboratories, Inc., Andover MA |
| Glutamine-13C5 | 322.1 | 171.1 | 2.33 | G597001. Toronto Research Chemicals, North York ON Canada |
| Glycine-13C2, 15N | 249.1 | 171.1 | 2.61 | MSK-A2-S. Cambridge Isotope Laboratories, Inc., Andover MA |
| Histidine-13C6, 15N3 | 335.1 | 165.1 | 0.99 | MSK-A2-S. Cambridge Isotope Laboratories, Inc., Andover MA |
| Isoleucine-13C6, 15N | 309.1 | 171.1 | 5.49 | MSK-A2-S. Cambridge Isotope Laboratories, Inc., Andover MA |
| Kynurenine-D6 | 385.2 | 171.1 | 5.55 | DLM-7842-0. Cambridge Isotope Laboratories, Inc., Andover MA |
| Leucine-13C6, 15N | 309.1 | 171.1 | 5.56 | MSK-A2-S. Cambridge Isotope Laboratories, Inc., Andover MA |
| Lysine-13C6, 15N | 248.1 | 171.1 | 4.31 | MSK-A2-S. Cambridge Isotope Laboratories, Inc., Andover MA |
| Methionine-13C5, 15N | 326.1 | 171.1 | 4.84 | MSK-A2-S. Cambridge Isotope Laboratories, Inc., Andover MA |
| Ornithine-D7 | 310.1 | 171.1 | 4.08 | D-7319. CDN Isotopes, Pointe-Claire, QC Canada |
| Phenylalanine-13C9, 15N | 346.1 | 171.1 | 5.70 | MSK-A2-S. Cambridge Isotope Laboratories, Inc., Andover MA |
| Proline-13C5, 15N | 292.1 | 171.1 | 3.98 | MSK-A2-S. Cambridge Isotope Laboratories, Inc., Andover MA |
| Sarcosine-D3 | 263.1 | 116.1 | 2.95 | S140502. Toronto Research Chemicals, North York ON Canada |
| Serine-13C3, 15N | 280.1 | 171.1 | 2.34 | MSK-A2-S. Cambridge Isotope Laboratories, Inc., Andover MA |
| Serotonin-D4 | 351.2 | 171.1 | 4.95 | S277982. Toronto Research Chemicals, North York ON Canada |
| Taurine-D4 | 300.1 | 171.1 | 2.31 | T007852. Toronto Research Chemicals, North York ON Canada |
| Threonine-13C4, 15N | 295.1 | 171.1 | 3.26 | MSK-A2-S. Cambridge Isotope Laboratories, Inc., Andover MA |
| Tryptophan-D3 | 378.1 | 171.1 | 5.79 | T947213. Toronto Research Chemicals, North York ON Canada |
| Tyrosine-13C9, 15N | 362.1 | 171.1 | 4.70 | MSK-A2-S. Cambridge Isotope Laboratories, Inc., Andover MA |
| Valine-13C5, 15N | 294.1 | 171.1 | 4.88 | MSK-A2-S. Cambridge Isotope Laboratories, Inc., Andover MA |

### Bioinformatic Analysis

The concentration values of each metabolite were log base 2 transformed and Z-score normalized prior to analyses. Analysis of covariance (ANCOVA) and pairwise Pearson correlation analysis were performed on each amine compound. False discovery rates were controlled for multiple testing using the Benjamini and Hochberg (27) method of p-value correction. A comparison was considered significant if the corrected p-value was less than 0.05. Dissimilarity measurements of 1−the absolute value of the Pearson correlation coefficients (p) was used to calculate distances for clustering. Wards' method was utilized for hierarchical clustering. Bootstrap analysis of the clustering result was performed using the pvclust package in R (28). Clusters were considered significant when the unbiased p-value was ≥0.95. The non-linear iterative partial least squares (NIPALS) algorithm was used for principal component analysis (PCA) and confidence intervals drawn at 95th percentile of the PCA scores using Hotelling's T2 statistic using the package PCAmethods (29). Welch T-tests were used to test for differences in study populations. The independence of subject metadata relative to the metabotypes was tested using the Fisher Exact test statistic with an alpha of 0.05 to reject the null hypothesis. These analyses were conducted with R (version 3.4.3).

### Establishing and Assessing Diagnostic Thresholds

A heuristic algorithm was developed to identify individual biomarkers able to discriminate ASD subpopulations, indicative of a metabotype, using a threshold for metabolite abundance or ratios. Diagnostic thresholds were established in the training set to generate a subpopulation with at least 10% of the ASD population while minimizing the number of TYP subjects in the subpopulation. A subject exceeding the diagnostic threshold was scored as a metabotype-positive ASD subject and the remaining subjects as metabotype-negative. Diagnostic performance metrics of specificity (detection of TYP), sensitivity (detection of ASD) and positive predictive value (PPV, percent of metabotype-positives that are ASD) were calculated based on metabotype status (positive or negative) and ADOS-2 diagnosis (ASD or TYP).

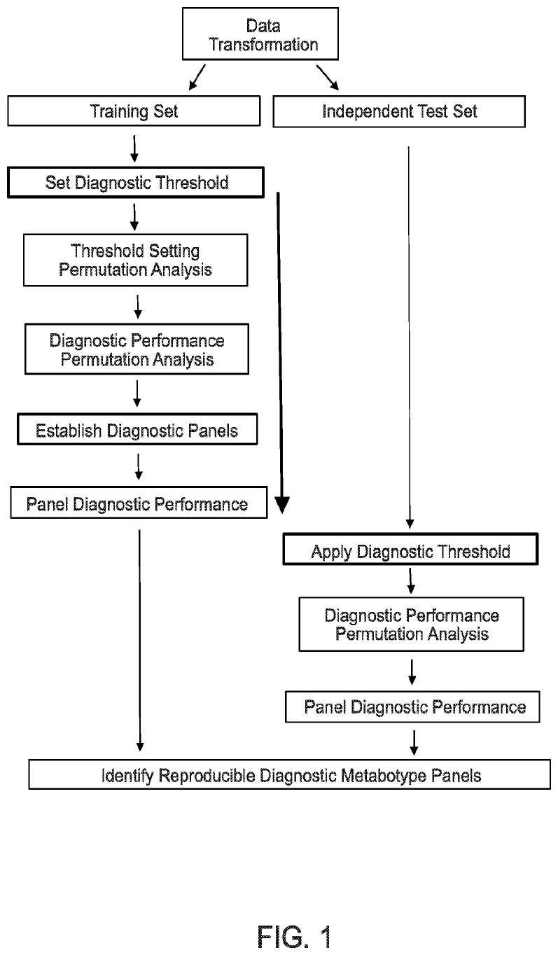

FIG. 1 is an outline of computational procedures utilized to set diagnostic thresholds and to evaluate diagnostic performance. Diagnostic thresholds were set for each metabolite or metabolite ratio in the training set and the threshold was applied to the training or test set to identify the affected subpopulation and determine the observed diagnostic performance. Permutation analysis was performed to evaluate the frequency at which the observed diagnostic performance occurred by chance. A diagnostic test was considered to identify a relevant metabolic subpopulation if the observed performance metrics occurred in less than 5% of 1000 iterations of random permutations of the subjects' diagnoses.

Permutation analysis was performed to test the probability that the observed diagnostic performance values from threshold setting and subpopulation prediction could be due to chance. Chance was assessed using 1000 permutations of subject diagnoses in the training set for threshold setting and subpopulation prediction or test set following subpopulation prediction. In both permutation procedures, the probability that observed biomarker performance metrics were due to chance was calculated based on the frequency that the observed sensitivity, specificity, and PPV were met or exceeded in the random permutation set.

When the diagnostic ratios were combined into panels of ratios to test for ASD associated metabotypes, the minimum performance required to consider a metabotype as reproducible were a sensitivity ≥5%, a specificity ≥95%, and a PPV≥90% in both training and test sets.

### Example 3: Results

### Children's Autism Metabolome Project (CAMP) Study Demographics

The training and test sets of subjects were chosen with appropriate power and randomization. The ASD prevalence, DQ, and gender composition between the training and test sets are equivalent (p value>0.05). However, the ASD population contains 16.5% more males than the TYP population (p value<0.01). The ASD population is slightly older than the TYP by 3.3 months (p value<0.01) and the ASD subjects within the training set are 1.4 months older than ASD subjects in the test set (p value<0.01).

### Analysis of Amine-Containing Metabolites Between ASD and TYP Study Populations

Analysis of covariance (ANCOVA) was performed on 31 amine containing metabolites in the training set of subjects to test the effect of gender or diagnoses controlling for subject age on metabolite means. No significant differences were identified in metabolite abundance values for diagnosis, age, sex, gender or their interactions. Analysis of covariance (ANCOVA) of diagnosis and sex controlling for subject age is shown in Table 7.

TABLE 7

| | Fold | | | | Age x | Age x | | Age x |
|---|---|---|---|---|---|---|---|---|
| | Change | Age | Diagnosis | Sex | Diagnosis | Sex | Diagnosis x | Diagnosis x |
| Metabolite | (ASD/TYP) | FDR | FDR | FDR | FDR | FDR | Sex FDR | Sex FDR |
| 4-Hydroxyproline (Hyp) | 0.992 | 0.814 | 0.991 | 1.000 | 0.984 | 0.978 | 0.954 | 0.980 |
| Alanine (Ala) | 1.104 | 0.987 | 0.898 | 0.982 | 0.984 | 0.978 | 0.963 | 0.980 |
| Arginine (Arg) | 1.013 | 0.920 | 0.898 | 0.982 | 0.984 | 0.978 | 0.954 | 0.980 |
| Asparagine (Asn) | 1.058 | 0.920 | 0.856 | 0.982 | 0.937 | 0.978 | 0.990 | 0.980 |
| Aspartate (Asp) | 1.123 | 0.215 | 0.605 | 0.908 | 0.937 | 0.871 | 0.954 | 0.980 |
| Citrulline (Cit) | 1.037 | 0.722 | 0.898 | 0.926 | 0.984 | 0.871 | 0.963 | 0.980 |
| Ethanolamine (ETA) | 1.102 | 0.920 | 0.605 | 0.908 | 0.768 | 0.906 | 0.954 | 0.980 |
| Glutamate (Glu) | 1.151 | 0.662 | 0.898 | 0.982 | 0.984 | 0.978 | 0.963 | 0.980 |
| Glutamine (Gln) | 1.019 | 0.978 | 0.889 | 0.982 | 0.982 | 0.978 | 0.954 | 0.980 |
| Glycine (Gly) | 1.1 | 0.920 | 0.898 | 0.982 | 0.984 | 0.978 | 0.963 | 0.980 |
| Histidine (His) | 1.026 | 0.536 | 0.266 | 0.908 | 0.456 | 0.871 | 0.954 | 0.980 |
| Homocitrulline (Hci) | 0.999 | 0.536 | 0.918 | 0.908 | 0.984 | 0.871 | 0.998 | 0.980 |
| Homoserine (Hse) | 1.03 | 0.814 | 0.898 | 1.000 | 0.982 | 0.978 | 0.954 | 0.980 |
| Isoleucine (Ile) | 1.019 | 0.920 | 0.898 | 0.982 | 0.984 | 0.978 | 0.954 | 0.980 |
| Kynurenine (Kyn) | 0.987 | 0.536 | 0.898 | 0.908 | 0.982 | 0.871 | 0.963 | 0.980 |
| Leucine (Leu) | 1.01 | 0.978 | 0.898 | 0.982 | 0.984 | 0.978 | 0.954 | 0.980 |
| Lysine (Lys) | 1.018 | 0.920 | 0.918 | 0.982 | 0.984 | 0.978 | 0.963 | 0.980 |
| Methionine (Met) | 1.032 | 0.920 | 0.889 | 0.982 | 0.937 | 0.978 | 0.963 | 0.980 |
| Ornithine (Orn) | 1.09 | 0.536 | 0.898 | 0.982 | 0.984 | 0.871 | 0.963 | 0.980 |
| Phenylalanine (Phe) | 0.995 | 0.920 | 0.898 | 0.908 | 0.984 | 0.871 | 0.954 | 0.980 |
| Proline (Pro) | 1.06 | 0.987 | 0.898 | 0.908 | 0.984 | 0.871 | 0.954 | 0.980 |
| Sarcosine (Sar) | 1.071 | 0.215 | 0.266 | 0.908 | 0.456 | 0.871 | 0.954 | 0.980 |
| Serine (Ser) | 1.044 | 0.665 | 0.575 | 0.908 | 0.768 | 0.950 | 0.954 | 0.980 |
| Taruine (Tau) | 1.206 | 0.920 | 0.838 | 0.908 | 0.982 | 0.871 | 0.954 | 0.980 |
| Threonine (Thr) | 1.028 | 0.662 | 0.898 | 0.908 | 0.982 | 0.871 | 0.954 | 0.980 |
| Tryptophan (Trp) | 1.022 | 0.536 | 0.889 | 0.929 | 0.937 | 0.871 | 0.963 | 0.980 |
| Tyrosine (Tyr) | 0.976 | 0.673 | 0.898 | 1.000 | 0.984 | 0.978 | 0.963 | 0.980 |
| Valine (Val) | 1.002 | 0.920 | 0.918 | 0.982 | 0.984 | 0.978 | 0.954 | 0.980 |
| β-alanine (bAla) | 1.048 | 0.920 | 0.898 | 0.908 | 0.984 | 0.871 | 0.963 | 0.980 |
| β-Aminoisobutyric Acid (BAIBA) | 0.982 | 0.536 | 0.918 | 0.929 | 0.984 | 0.871 | 0.954 | 0.980 |
| γ-Aminobutyric acid (GABA) | 1.087 | 0.150 | 0.266 | 0.908 | 0.456 | 0.871 | 0.710 | 0.346 |

Abbreviations: TYP, Typically Developing; ASD Autism Spectrum Disorder.
FDR, false discovery rate corrected p-value from ANCOVA analysis.
FDR < 0.05 considered statistically significant.
An “x” in a column indicates an interaction of factors.

These results suggest that within the demographic ranges in this study, the differences in subject age or sex have little impact on metabolite abundance. Therefore, the differences in the composition of ASD and TYP study populations are unlikely to have significant impact on study results.
Metabolite Correlations within ASD Reveal Distinct Clusters of Amine Metabolites

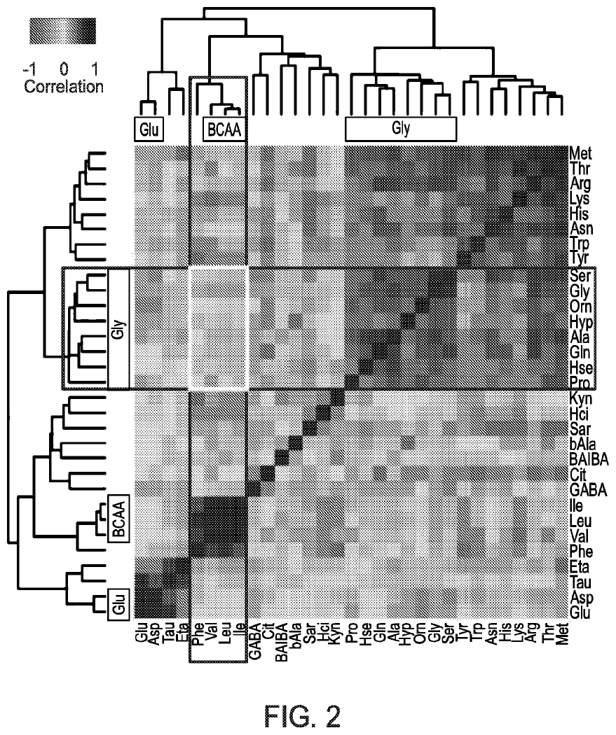

We then examined the relationship among the amine metabolites in the training set of ASD subjects by pairwise Pearson correlation analysis and hierarchical clustering to identify metabolites with co-regulated metabolism. Three clusters of metabolites with positive correlations were identified. Cluster 1 contains the metabolites serine, glycine, ornithine, 4-hydroxyproline, alanine, glutamine, homoserine, and proline (i.e., the glycine cluster—mean ρ 0.45±0.02). Cluster 2 contains the BCAAs (leucine, isoleucine, and valine) and phenylalanine where the BCAAs are more highly correlated with each other (mean pairwise p of 0.86±0.02) than the BCAAs are with phenylalanine (mean pairwise p of 0.56±0.02) (i.e., the BCAA cluster red boxes, FIG. 2. Cluster 3 contains glutamate and aspartate (i.e., the glutamate cluster—p of 0.78, FIG. 2. The intersection of the glycine and BCAA clusters yielded a block of negative correlations (FIG. 2, intersection of boxes). We decided to focus our analysis on the glycine cluster metabolites that are negatively correlated with BCAA metabolites. Proline was removed from further analysis because it was not negatively correlated with the BCAAs. Phenylalanine was removed because it is not a BCAA metabolite.

FIG. 2 is a heat map with hierarchical clustering dendrograms from pairwise Pearson correlations of metabolite abundances for the training set ASD subjects. Red filled boxes associated with the dendrograms identify statistically significant clusters following bootstrap resampling. The names of these clusters appear within the red boxes. The green open boxes highlight the BCAA cluster in the columns and the glycine cluster in the rows. The intersection of the two green boxes, marked by a yellow open rectangle, identifies the block of negative correlations shared by the glycine and BCAA clusters. Abbreviations: BCAA, branched chain amino acids; BAIBA, β-Aminoisobutyric Acid; GABA, γ-Aminobutyric acid, bAla, β-alanine; Hci, Homocitrulline; Hse, Homoserine; ETA, Ethanolamine; Sar, Sarcosine; Tau, Taurine; Hyp, 4-Hydroxyproline; Cit, Citrulline

Identification of Amino Acid:BCAA Imbalance Metabotypes Associated with ASD

The negative correlation between the BCAA and glycine cluster led us to evaluate ratios of these AA as predictors of ASD diagnosis. Ratios can uncover biological properties not evident with individual metabolites and increase the signal when two metabolites with a negative correlation are evaluated. This strategy, for example, formed the basis of the standard phenylketonuria (PKU) diagnostic using a ratio of phenylalanine and tyrosine (30). Based on analysis of boxplots, we created ratios with one of the BCAAs in the denominator and one of the glycine cluster metabolites in the numerator. Thresholds for the ratios were set in the training set and evaluated in the test set of subjects. The BCAA ratios of glutamine, glycine, ornithine and serine identified subpopulations of subjects associated with an ASD diagnosis at a rate higher than chance in both training and test sets. Diagnostic performance of the branched chain amino acid (BCAA) ratios used in development of the Amino Acid Dysregulation Metabotypes (AADM) diagnostic panels as measured in the training set of subjects are shown in Table 8.

TABLE 8

| Metabolite | Observed Training Set Confusion Matrix Metrics | | | | | | | | Permutation | |
|---|---|---|---|---|---|---|---|---|---|---|
| Ratio | TP | FP | TN | FN | N | SEN | SPEC | PPV | Thresh | Pred |
| Ala:Ile | 51 | 9 | 76 | 202 | 338 | 0.202 | 0.894 | 0.850 | 1.22E−02 | 3.40E−02 |
| Ala:Leu | 62 | 9 | 76 | 191 | 338 | 0.245 | 0.894 | 0.873 | 4.00E−04 | 2.00E−03 |
| Ala:Val | 45 | 7 | 78 | 208 | 338 | 0.178 | 0.918 | 0.865 | 1.04E−02 | 2.20E−02 |
| Gln:Ile | 32 | 2 | 83 | 221 | 338 | 0.126 | 0.976 | 0.941 | 1.60E−03 | 3.00E−03 |
| Gln:Leu | 26 | 2 | 83 | 227 | 338 | 0.103 | 0.976 | 0.929 | 1.42E−02 | 9.00E−03 |
| Gln:Val | 33 | 2 | 83 | 220 | 338 | 0.130 | 0.976 | 0.943 | 1.20E−03 | 1.00E−03 |
| Gly:Ile | 44 | 4 | 81 | 209 | 338 | 0.174 | 0.953 | 0.917 | 1.40E−03 | 0.00E+00 |
| Gly:Leu | 37 | 4 | 81 | 216 | 338 | 0.146 | 0.953 | 0.902 | 5.80E−03 | 8.00E−03 |
| Gly:Val | 32 | 2 | 83 | 221 | 338 | 0.126 | 0.976 | 0.941 | 2.80E−03 | 3.00E−03 |
| Hse:Ile | 16 | 2 | 83 | 237 | 338 | 0.063 | 0.976 | 0.889 | 9.34E−02 | 1.16E−01 |
| Hse:Leu | 27 | 4 | 81 | 226 | 338 | 0.107 | 0.953 | 0.871 | 7.44E−02 | 8.20E−02 |
| Hse:Val | 17 | 3 | 82 | 236 | 338 | 0.067 | 0.965 | 0.850 | 2.16E−01 | 2.09E−01 |
| Orn:Ile | 29 | 3 | 82 | 224 | 338 | 0.115 | 0.965 | 0.906 | 1.48E−02 | 2.10E−02 |
| Orn:Leu | 26 | 3 | 82 | 227 | 338 | 0.103 | 0.965 | 0.897 | 3.78E−02 | 3.70E−02 |
| Orn:Val | 30 | 4 | 81 | 223 | 338 | 0.119 | 0.953 | 0.882 | 3.16E−02 | 3.70E−02 |
| Ser:Ile | 33 | 4 | 81 | 220 | 338 | 0.130 | 0.953 | 0.892 | 1.40E−02 | 2.00E−02 |
| Ser:Leu | 35 | 5 | 80 | 218 | 338 | 0.138 | 0.941 | 0.875 | 2.16E−02 | 3.00E−02 |
| Ser:Val | 48 | 5 | 80 | 205 | 338 | 0.190 | 0.941 | 0.906 | 6.00E−04 | 1.00E−03 |
| Hyp:Ile | 28 | 5 | 80 | 225 | 338 | 0.111 | 0.941 | 0.848 | 1.16E−01 | 1.14E−01 |
| Hyp:Leu | 29 | 7 | 78 | 224 | 338 | 0.115 | 0.918 | 0.806 | 2.82E−01 | 2.69E−01 |
| Hyp:Val | 61 | 11 | 74 | 192 | 338 | 0.241 | 0.871 | 0.847 | 5.40E−03 | 1.70E−02 |

The diagnostic thresholds were set in the training set of samples. For each ratio permutation columns contain the frequency that the observed training set performance metrics of sensitivity, specificity, and positive predictive value were exceeding in 1000 random permutations of the subjects' diagnoses. The Thresh column contains frequency a diagnostic threshold set in permutation analysis met or exceed the performance metrics observed with the threshold set in training set. The Pred column contains the frequency that the diagnostic threshold set in the 5 training set identified a subpopulation in permutation analysis that met or exceeded the performance metrics observed in the training set.
Abbreviations: TP, True positive; FP, False Negative; TN, True Negative; N, Total Subjects, SEN, Sensitivity; SPEC, Specificity; PPV, Positive predictive value.
Performance metrics of the diagnostic branched chain amino acid (BCAA) ratios used to identify subpopulations of ASD in the test set of subjects are shown in Table 9.

TABLE 9

| | | | | | | | | | Permuta- |
|---|---|---|---|---|---|---|---|---|---|
| Metabolite | Observed Test Set Confusion Matrix Metrics | | | | | | | | tion |
| Ratio | TP | FP | TN | FN | N | SEN | SPEC | PPV | Pred |
| Ala:Ile | 43 | 5 | 74 | 220 | 342 | 0.163 | 0.937 | 0.896 | 1.30E−02 |
| Ala:Leu | 57 | 9 | 70 | 206 | 342 | 0.217 | 0.886 | 0.864 | 2.40E−02 |
| Ala:Val | 49 | 9 | 70 | 214 | 342 | 0.186 | 0.886 | 0.845 | 9.90E−02 |
| Gln:Ile | 40 | 3 | 76 | 223 | 342 | 0.152 | 0.962 | 0.930 | 2.00E−03 |
| Gln:Leu | 39 | 1 | 78 | 224 | 342 | 0.148 | 0.987 | 0.975 | 0.00E+00 |
| Gln:Val | 42 | 5 | 74 | 221 | 342 | 0.160 | 0.937 | 0.894 | 1.50E−02 |
| Gly:Ile | 46 | 5 | 74 | 217 | 342 | 0.175 | 0.937 | 0.902 | 9.00E−03 |
| Gly:Leu | 35 | 5 | 74 | 228 | 342 | 0.133 | 0.937 | 0.875 | 5.50E−02 |
| Gly:Val | 30 | 6 | 73 | 233 | 342 | 0.114 | 0.924 | 0.833 | 2.22E−01 |
| Hse:Ile | 27 | 1 | 78 | 236 | 342 | 0.103 | 0.987 | 0.964 | 9.00E−03 |
| Hse:Leu | 53 | 4 | 75 | 210 | 342 | 0.202 | 0.949 | 0.930 | 0.00E+00 |
| Hse:Val | 37 | 5 | 74 | 226 | 342 | 0.141 | 0.937 | 0.881 | 4.30E−02 |
| Orn:Ile | 36 | 4 | 75 | 227 | 342 | 0.137 | 0.949 | 0.900 | 2.50E−02 |
| Orn:Leu | 32 | 2 | 77 | 231 | 342 | 0.122 | 0.975 | 0.941 | 3.00E−03 |
| Orn:Val | 42 | 3 | 76 | 221 | 342 | 0.160 | 0.962 | 0.933 | 4.00E−03 |
| Ser:Ile | 34 | 4 | 75 | 229 | 342 | 0.129 | 0.949 | 0.895 | 2.20E−02 |
| Ser:Leu | 44 | 6 | 73 | 219 | 342 | 0.167 | 0.924 | 0.880 | 3.80E−02 |
| Ser:Val | 60 | 10 | 69 | 203 | 342 | 0.228 | 0.873 | 0.857 | 3.90E−02 |
| Hyp:Ile | 23 | 7 | 72 | 240 | 342 | 0.087 | 0.911 | 0.767 | 6.03E−01 |
| Hyp:Leu | 25 | 8 | 71 | 238 | 342 | 0.095 | 0.899 | 0.758 | 6.64E−01 |
| Hyp:Val | 56 | 18 | 61 | 207 | 342 | 0.213 | 0.772 | 0.757 | 6.64E−01 |

Diagnostic thresholds were set in the training set subjects. For each ratio, permutation columns contain the frequency that the observed training set performance metrics of sensitivity, specificity, and positive predictive value were met or exceeded in 1000 random permutations of the subjects' diagnoses. The Pred column contains the frequency that the diagnostic threshold set in the training set identified a subpopulation in the permutation analysis that met or exceeded the performance metrics observed in the training set.
Abbreviations: TP. True positive; FP, False positive; TN, True Negative; FN, False Negative; N, Total Subjects; SEN, Sensitivity; SPEC, Specificity; PPV, Positive Predictive Value.

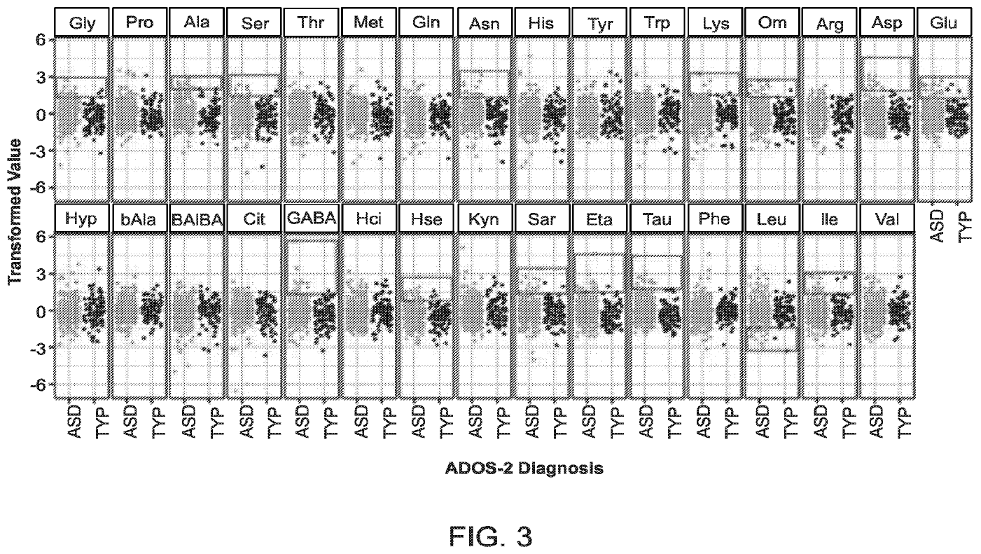

FIG. 3 is a scatter plot of the training set's transformed amine concentration values. Blue boxes indicate groups that are comprised of greater than 90% ASD subjects (90% PPV). These groups include at least 5% of the training set of ASD subjects (5% sensitivity). Glycine, alanine, asparagine, aspartic acid, GABA, glutamic acid, homoserine, ethanolamine, sarcosine, serine and taurine exhibited elevated metabolite levels in ASD subjects, while leucine exhibited decreased metabolite levels in ASD subjects. Red=ASD, Black=TYP; TYP, Typically Developing; ASD, Autism Spectrum Disorder. BAIBA, β-Aminoisobutyric Acid; GABA, γ-Aminobutyric acid, bAla, β-alanine; Hci, Homocitrulline; Hse, Homoserine; ETA, Ethanolamine; Sar, Sarcosine; Tau, Taurine; Hyp, 4-Hydroxyproline; Cit, Citrulline.

Table 10 shows diagnostic performance metrics of amine ratios to discriminate subpopulations of ASD subjects in the training and test sets of subjects.

TABLE 10

| | Sensitivity | | Specificity | | Pos. Pred. Value | | Permutation Test | |
|---|---|---|---|---|---|---|---|---|
| Ratio | Train | Test | Train | Test | Train | Test | Train | Test |

Ratios used to create AADM_{Alanine}

| | | | | | | | | |
|---|---|---|---|---|---|---|---|---|
| Ala:Ile | 0.202 | 0.163 | 0.894 | 0.937 | 0.850 | 0.896 | 3.40E−02 | 1.30E−02 |
| Ala:Leu | 0.245 | 0.217 | 0.894 | 0.886 | 0.873 | 0.864 | 2.00E−03 | 2.40E−02 |
| Ala:Val | 0.178 | 0.186 | 0.918 | 0.886 | 0.865 | 0.845 | 2.20E−02 | 9.90E−02 |

Ratios used to create AADM_{Glutamine}

| | | | | | | | | |
|---|---|---|---|---|---|---|---|---|
| Gln:Ile^{a} | 0.126 | 0.152 | 0.976 | 0.962 | 0.941 | 0.930 | 3.00E−03 | 2.00E−03 |
| Gln:Leu^{a} | 0.103 | 0.148 | 0.976 | 0.987 | 0.929 | 0.975 | 9.00E−03 | 0.00E+00 |
| Gln:Val | 0.130 | 0.160 | 0.976 | 0.937 | 0.943 | 0.894 | 1.00E−03 | 1.50E−02 |

Ratios used to create AADM_{Glycine}

| | | | | | | | | |
|---|---|---|---|---|---|---|---|---|
| Gly:Ile^{a} | 0.174 | 0.175 | 0.953 | 0.937 | 0.917 | 0.902 | 0.00E+00 | 9.00E−03 |
| Gly:Leu | 0.146 | 0.133 | 0.953 | 0.937 | 0.902 | 0.875 | 8.00E−03 | 5.50E−02 |
| Gly:Val | 0.126 | 0.114 | 0.976 | 0.924 | 0.941 | 0.833 | 3.00E−03 | 2.22E−01 |

Ratios used to create AADM_{Homoserine}

| | | | | | | | | |
|---|---|---|---|---|---|---|---|---|
| Hse:Ile | 0.063 | 0.103 | 0.976 | 0.949 | 0.889 | 0.964 | 1.16E−01 | 9.00E−03 |
| Hse:Leu | 0.107 | 0.202 | 0.953 | 0.975 | 0.871 | 0.930 | 8.20E−02 | 0.00E+00 |
| Hse:Val | 0.067 | 0.141 | 0.965 | 0.962 | 0.850 | 0.881 | 2.09E−01 | 4.30E−02 |

Ratios used to create AADM_{Ornithine}

| | | | | | | | | |
|---|---|---|---|---|---|---|---|---|
| Orn:Ile^{a} | 0.115 | 0.137 | 0.965 | 0.949 | 0.906 | 0.900 | 2.10E−02 | 2.50E−02 |
| Orn:Leu^{a} | 0.103 | 0.122 | 0.965 | 0.975 | 0.897 | 0.941 | 3.70E−02 | 3.00E−03 |
| Orn:Val | 0.119 | 0.160 | 0.953 | 0.962 | 0.882 | 0.933 | 3.70E−02 | 4.00E−03 |

Ratios used to create AADM_{Serine}

| | | | | | | | | |
|---|---|---|---|---|---|---|---|---|
| Ser:Ile | 0.130 | 0.129 | 0.953 | 0.949 | 0.892 | 0.895 | 2.00E−02 | 2.20E−02 |
| Ser:Leu | 0.138 | 0.167 | 0.941 | 0.924 | 0.875 | 0.880 | 3.00E−02 | 3.80E−02 |
| Ser:Val | 0.190 | 0.228 | 0.941 | 0.873 | 0.906 | 0.857 | 1.00E−03 | 3.90E−02 |

Ratios used to create AADM_{Hydroxyproline}

| | | | | | | | | |
|---|---|---|---|---|---|---|---|---|
| Hyp:Ile | 0.111 | 0.087 | 0.941 | 0.911 | 0.848 | 0.767 | 1.14E−01 | 6.03E−01 |
| Hyp:Leu | 0.115 | 0.095 | 0.918 | 0.899 | 0.806 | 0.758 | 2.69E−01 | 6.64E−01 |
| Hyp:Val | 0.241 | 0.213 | 0.871 | 0.772 | 0.847 | 0.757 | 1.70E−02 | 6.64E−01 |

The ratios all include branched chain amino acid (BCAA) values in the denominators and negatively correlated Gly-cluster metabolites in the numerator.
Abbreviations: Pos., positive, Pred., predictive; AADM, amino acid dysregulation metabotype; ASD, autism spectrum disorder; Train, training set; Test, test set.
^{a}The observed diagnostic performance occurred in less than 5% of 1000 permutations of subject diagnosis in both training and test sets.

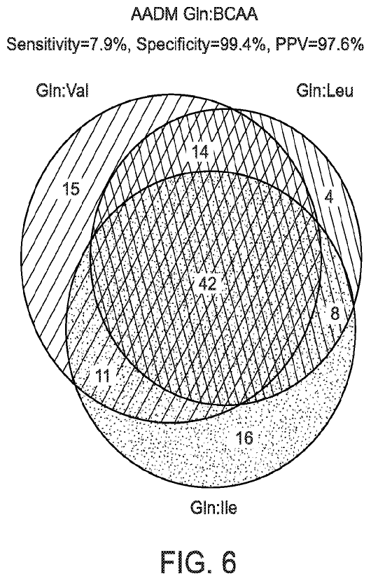

The correlation of the BCAAs with each other (p=0.86±0.02) and the overlap of affected-subjects (FIGS. 6, 9, and 12) identified by the AA:BCAA ratios suggested that a combination of ratios containing a single numerator and each of the three BCAAs as denominators could uncover BCAA metabolic dysregulation. Exploiting the positive correlation among the BCAAs in this way improves the specificity and PPV. For example, each of the Glycine:BCAA ratios (i.e. glycine:leucine or glycine:valine or glycine:isoleucine) results in a specificity of 94.1% and PPV of 91.1%. Comparison of the confusion matrix performance metrics of branched chain amino acid (BCAA) metabotypes created in the training set is shown in Table 11.

TABLE 11

| Amino Acid | | | | | | |
|---|---|---|---|---|---|---|
| Dysregulation | | | | | | |
| Metabotype | SEN | | SPEC | | PPV | |
| Diagnostic | Inter | Union | Inter | Union | Inter | Union |
| Ala:BCAA | 0.150 | 0.265 | 0.929 | 0.882 | 0.864 | 0.870 |
| Gln:BCAA | 0.079 | 0.174 | 0.988 | 0.965 | 0.952 | 0.936 |
| Gly:BCAA | 0.095 | 0.202 | 0.988 | 0.941 | 0.960 | 0.911 |
| Hse:BCAA | 0.036 | 0.123 | 0.988 | 0.941 | 0.900 | 0.861 |
| Orn:BCAA | 0.079 | 0.150 | 0.976 | 0.941 | 0.909 | 0.884 |
| Ser:BCAA | 0.091 | 0.237 | 0.965 | 0.918 | 0.885 | 0.896 |
| Hyp:BCAA | 0.087 | 0.245 | 0.965 | 0.871 | 0.880 | 0.849 |

Each metabotype used with the intersection or union of metabotype positive calls to predict as being metabotype positive.
Abbreviations: Inter, Intersection; SEN, Sensitivity; SPEC, Specificity; PPV, Positive predictive value.
However, requiring that the subject be positive for all three Glycine:BCAA ratios, results in a specificity of 98.8% and PPV of 96.0%. Through this process, we identified groups of subjects that exhibited an Amino Acid Dysregulation Metabotype (AADM). Subjects were identified by AADM when they exceeded an established threshold for all three AA:BCAA ratios. Since the nomenclature for these biomarkers can quickly become confusing, we have designated different AADMs using the numerator metabolite e.g. AADM_{glutamine}. Not all ratios of AAs to BCAA resulted in diagnostic differences between the ASD and TYP groups. We focused, therefore, on those AA:BCAA ratios that had the greatest predictive power including glutamine AADM (AADM_{glutamine}), glycine AADM (AADM_{glycine}) and ornithine AADM (AADMₒᵣₙᵢₜₕᵢₙₑ).

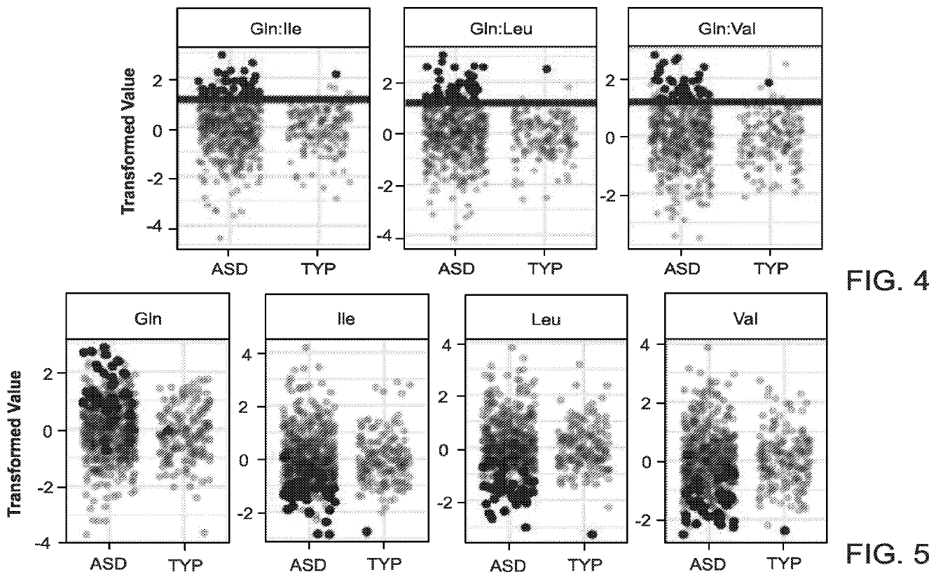

FIG. 4 shows scatter plots of ratios of levels of glutamine to various branched chain amino acids (BCAAs) in subjects with Autism Spectrum Disorder (ASD) and in typically developing subjects (TYP). Scatter plots of the ratios were used to create a glutamine amino acid dysregulation metabotype (AADM_{glutamine}). Red points represent AADM_{glutamine} positive subjects, and black points represent AADM_{glutamine} negative subjects. The red horizontal line is the diagnostic threshold set in the training set.

FIG. 5 shows scatter plots of levels of individual amino acids in ASD subjects and TYP subjects. Red points represent AADM_{glutamine} positive subjects, and black points represent AADM_{glutamine} negative subjects.

FIG. 6 is a Venn diagram of metabotype-positive subjects identified by the three ratios used for AADM_{glutamine}. Each circle represents the subjects identified by the diagnostic threshold for a given ratio. The intersection of the Venn diagram indicates the subjects called AADM_{glutamine} positive (red dots in scatter plots). Performance metrics above the Venn diagram represent entire study population (training and test sets).

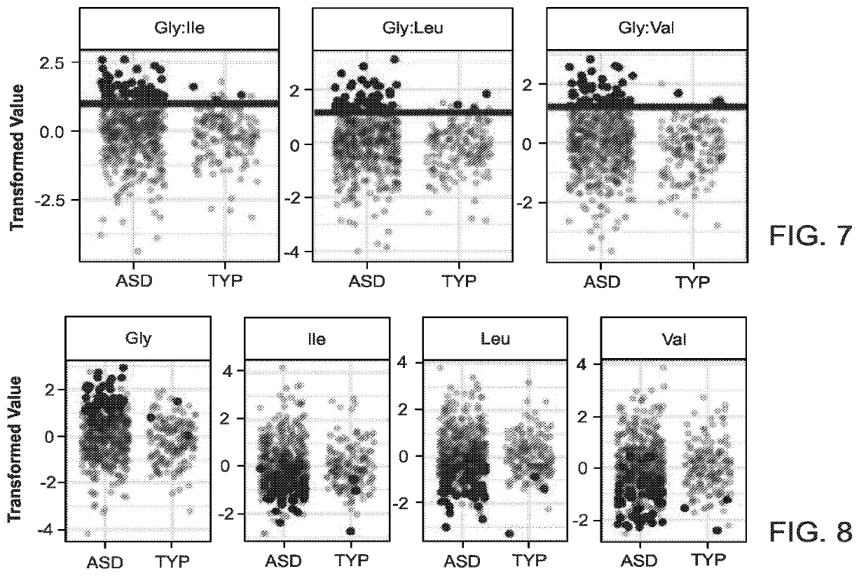

FIG. 7 shows scatter plots of ratios of levels of glycine to various branched chain amino acids in ASD subjects and TYP subjects. Scatter plots of the ratios were used to create an AADM_{glycine}. Red points represent AADM_{glycine} positive subjects, and black points represent AADM_{glycine} negative subjects. The red horizontal line is the diagnostic threshold set in the training set.

FIG. 8 shows scatter plots of levels of individual amino acids in ASD subjects and TYP subjects. Red points represent AADM_{glycine} positive subjects, and black points represent AADM_{glycine} negative subjects.

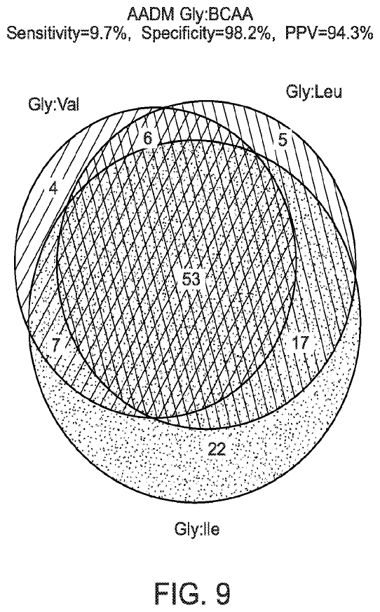

FIG. 9 is a Venn diagram of metabotype-positive subjects identified by the three ratios used for AADM_{glycine}. Each circle represents the subjects identified by the diagnostic threshold for a given ratio. The intersection of the Venn diagram indicates the subjects called AADM_{glycine} positive (red dots in scatter plots). Performance metrics above the Venn diagram represent entire study population (training and test sets).

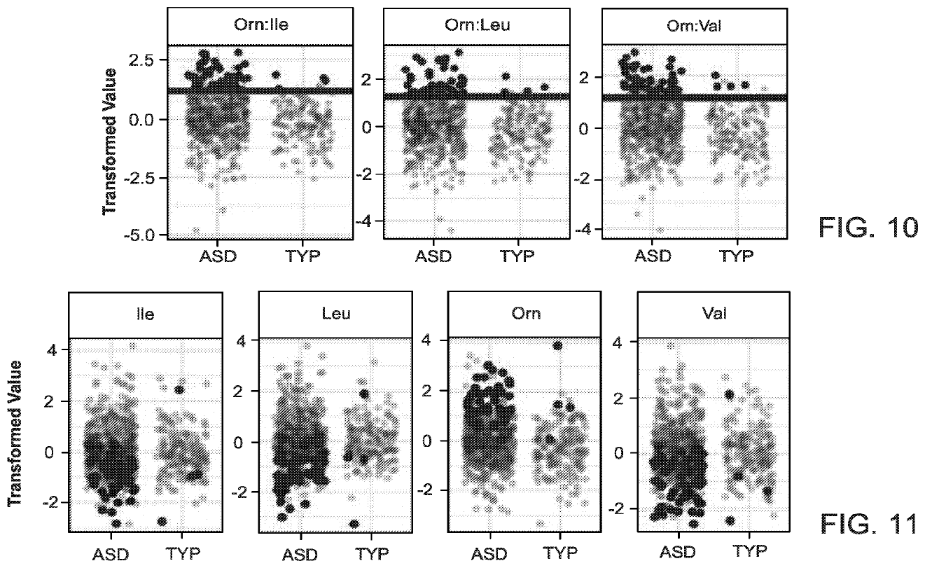

FIG. 10 shows scatter plots of ratios of levels of ornithine to various branched chain amino acids in ASD subjects and TYP subjects. Scatter plots of the ratios were used to create an AADMₒᵣₙᵢₜₕᵢₙₑ. Red points represent AADMₒᵣₙᵢₜₕᵢₙₑ positive subjects, and black points represent AADMₒᵣₙᵢₜₕᵢₙₑ negative subjects. The red horizontal line is the diagnostic threshold set in the training set.

FIG. 11 shows scatter plots of levels of individual amino acids in ASD subjects and TYP subjects. Red points represent AADMₒᵣₙᵢₜₕᵢₙₑ positive subjects, and black points represent AADMₒᵣₙᵢₜₕᵢₙₑ negative subjects.

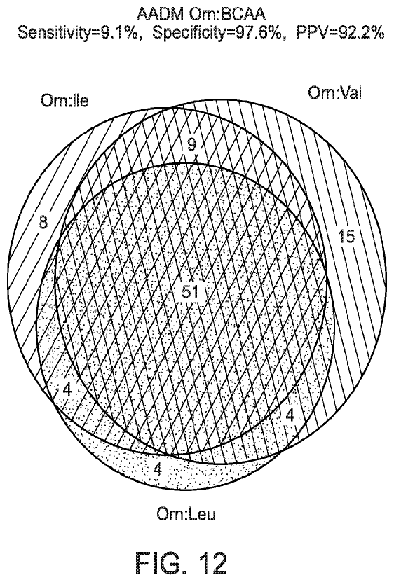

FIG. 12 is a Venn diagram of metabotype-positive subjects identified by the three ratios used for AADMₒᵣₙᵢₜₕᵢₙₑ. Each circle represents the subjects identified by the diagnostic threshold for a given ratio. The intersection of the Venn diagram indicates the subjects called AADMₒᵣₙᵢₜₕᵢₙₑ positive (red dots in scatter plots). Performance metrics above the Venn diagram represent entire study population (training and test sets).

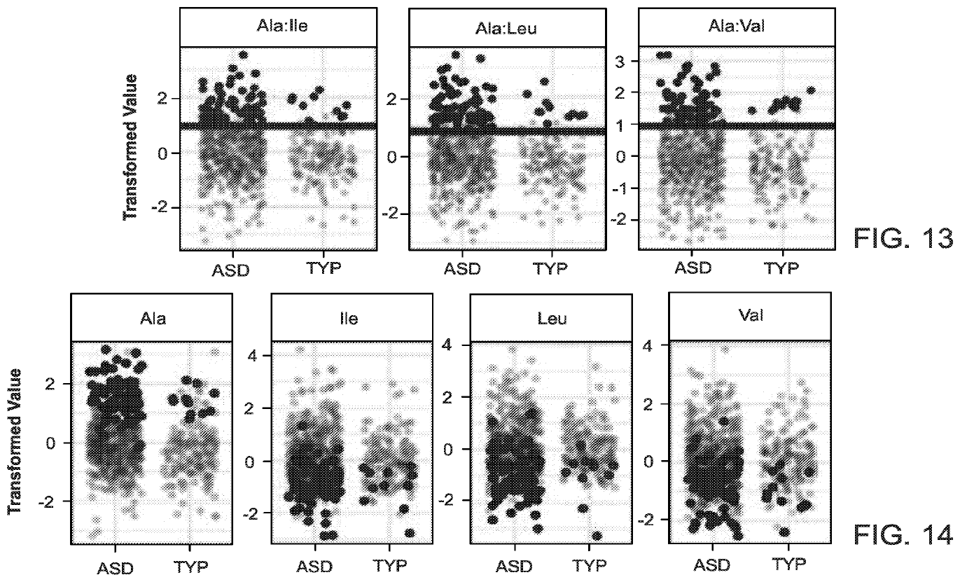

FIG. 13 shows scatter plots of ratios of levels of alanine to various branched chain amino acids in ASD subjects and TYP subjects. Scatter plots of the ratios were used to create an AADMₐₗₐₙᵢₙₑ. Red points represent AADMₐₗₐₙᵢₙₑ positive subjects, and black points represent AADMₐₗₐₙᵢₙₑ negative subjects. The red horizontal line is the diagnostic threshold set in the training set.

FIG. 14 shows scatter plots of levels of individual amino acids in ASD subjects and TYP subjects. Red points represent AADMₐₗₐₙᵢₙₑ positive subjects, and black points represent AADMₐₗₐₙᵢₙₑ negative subjects.

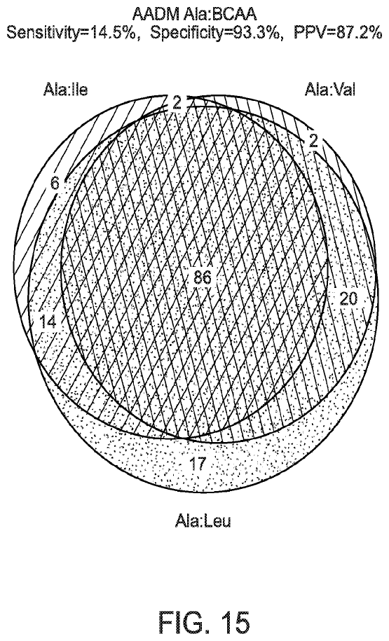

FIG. 15 is a Venn diagram of metabotype-positive subjects identified by the three ratios used for AADMₐₗₐₙᵢₙₑ. Each circle represents the subjects identified by the diagnostic threshold for a given ratio. The intersection of the Venn diagram indicates the subjects called AADMₐₗₐₙᵢₙₑ positive (red dots in scatter plots). Performance metrics above the Venn diagram represent entire study population (training and test sets).

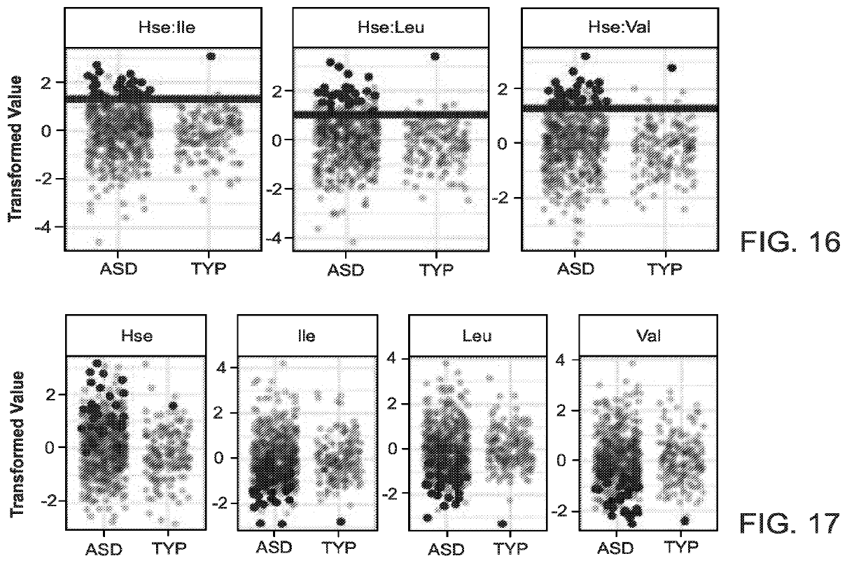

FIG. 16 shows scatter plots of ratios of levels of homoserine to various branched chain amino acids in ASD subjects and TYP subjects. Scatter plots of the ratios were used to create an AADMₕₒₘₒₛₑᵣᵢₙₑ. Red points represent AADMₕₒₘₒₛₑᵣᵢₙₑ positive subjects, and black points represent AADMₕₒₘₒₛₑᵣᵢₙₑ negative subjects. The red horizontal line is the diagnostic threshold set in the training set.

FIG. 17 shows scatter plots of levels of individual amino acids in ASD subjects and TYP subjects. Red points represent AADMₕₒₘₒₛₑᵣᵢₙₑ positive subjects, and black points represent AADMₕₒₘₒₛₑᵣᵢₙₑ negative subjects.

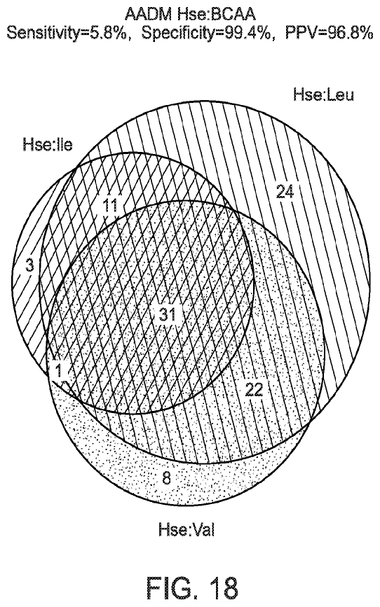

FIG. 18 is a Venn diagram of metabotype-positive subjects identified by the three ratios used for AADMₕₒₘₒₛₑᵣᵢₙₑ. Each circle represents the subjects identified by the diagnostic threshold for a given ratio. The intersection of the Venn diagram indicates the subjects called AADMₕₒₘₒₛₑᵣᵢₙₑ positive (red dots in scatter plots). Performance metrics above the Venn diagram represent entire study population (training and test sets).

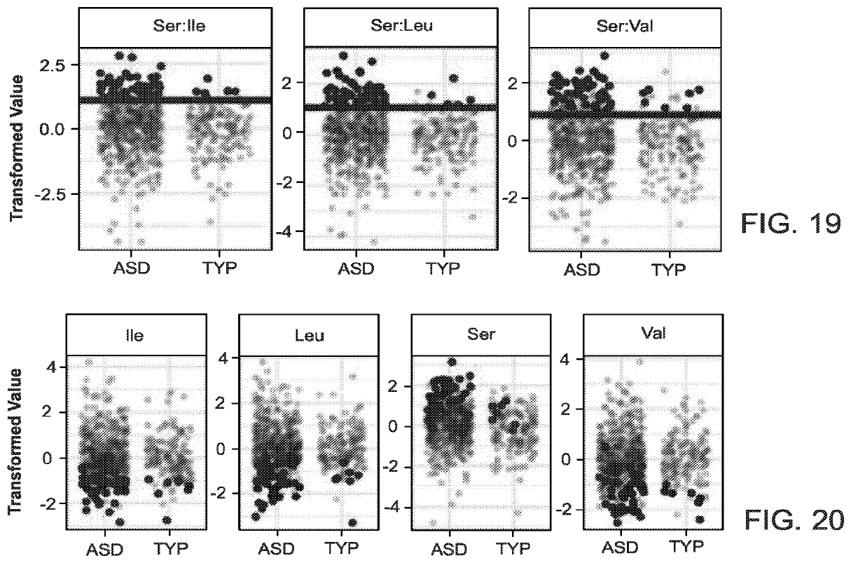

FIG. 19 shows scatter plots of ratios of levels of serine to various branched chain amino acids in ASD subjects and TYP subjects. Scatter plots of the ratios were used to create an AADMₛₑᵣᵢₙₑ. Red points represent AADMₛₑᵣᵢₙₑ positive subjects, and black points represent AADMₛₑᵣᵢₙₑ negative subjects. The red horizontal line is the diagnostic threshold set in the training set.

FIG. 20 shows scatter plots of levels of individual amino acids in ASD subjects and TYP subjects. Red points represent AADMₛₑᵣᵢₙₑ positive subjects, and black points represent AADMₛₑᵣᵢₙₑ negative subjects.

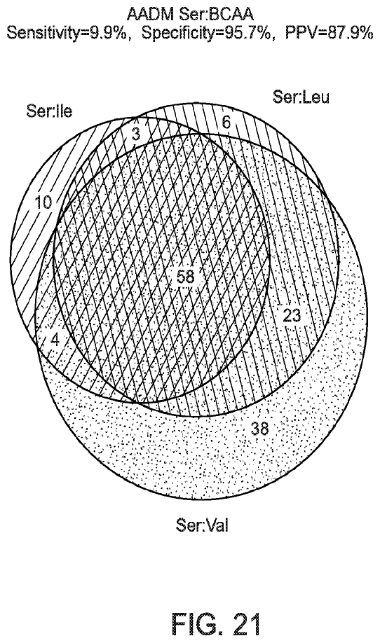

FIG. 21 is a Venn diagram of metabotype-positive subjects identified by the three ratios used for AADMₛₑᵣᵢₙₑ. Each circle represents the subjects identified by the diagnostic threshold for a given ratio. The intersection of the Venn diagram indicates the subjects called AADMₛₑᵣᵢₙₑ positive (red dots in scatter plots). Performance metrics above the Venn diagram represent entire study population (training and test sets).

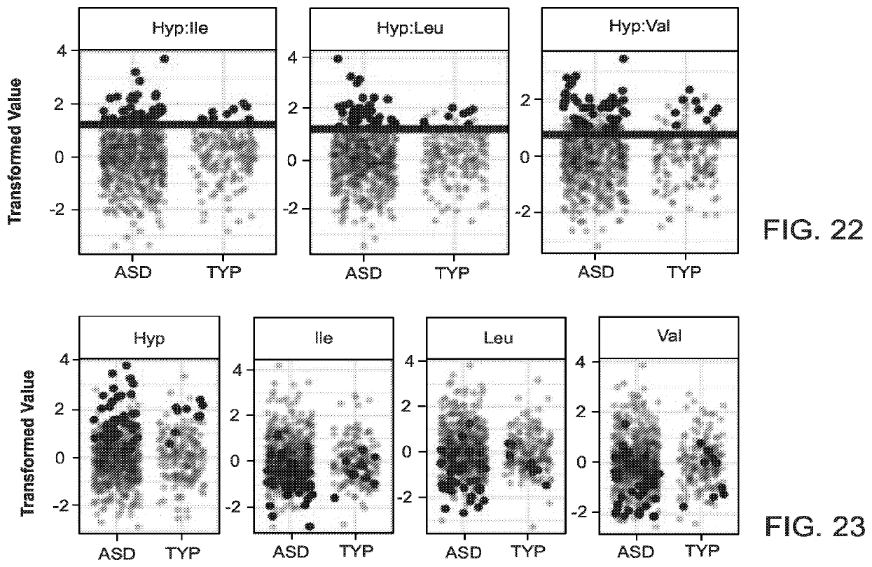

FIG. 22 shows scatter plots of ratios of levels of 4-hydroxyproline to various branched chain amino acids in ASD subjects and TYP subjects. Scatter plots of the ratios were used to create an AADM_{hydroxproline}. Red points represent AADM_{hydroxproline} positive subjects, and black points represent AADM_{hydroxproline} negative subjects. The red horizontal line is the diagnostic threshold set in the training set.

FIG. 23 shows scatter plots of levels of individual amino acids in ASD subjects and TYP subjects. Red points represent AADM_{hydroxproline} positive subjects, and black points represent AADM_{hydroxproline} negative subjects.

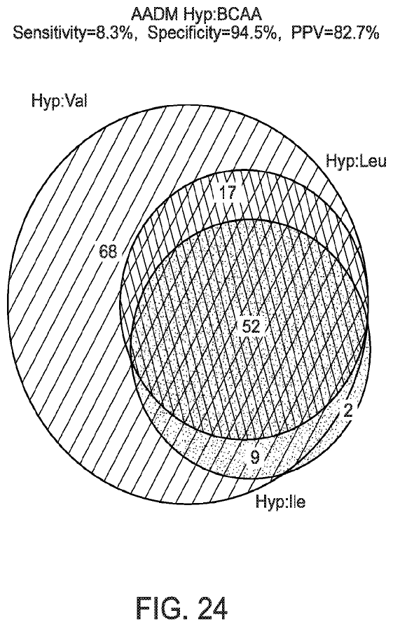

FIG. 24 is a Venn diagram of metabotype-positive subjects identified by the three ratios used for AADM hydroxproline. Each circle represents the subjects identified by the diagnostic threshold for a given ratio. The intersection of the Venn diagram indicates the subjects called AADM_{hydroxproline} positive (red dots in scatter plots). Performance metrics above the Venn diagram represent entire study population (training and test sets).

Table 12 shows diagnostic performance metrics of Amino Acid Dysregulation Metabotypes (AADM).

TABLE 12

| AADM | Sensitivity | | Specificity | | Pos. Pred. Value | |
|---|---|---|---|---|---|---|
| Diagnostic | Train | Test | Train | Test | Train | Test |
| Ala:BCAA | 0.150 | 0.141 | 0.929 | 0.937 | 0.884 | 0.881 |
| Gln:BCAA^{a} | 0.079 | 0.080 | 0.988 | 1.000 | 0.952 | 1.000 |
| Gly:BCAA^{a} | 0.095 | 0.099 | 0.988 | 0.975 | 0.960 | 0.929 |
| Hse:BCAA | 0.036 | 0.080 | 0.988 | 1.000 | 0.900 | 1.000 |
| Orn:BCAA^{a} | 0.078 | 0.103 | 0.976 | 0.975 | 0.909 | 0.931 |
| Ser:BCAA | 0.091 | 0.106 | 0.965 | 0.949 | 0.885 | 0.875 |
| Hyp:BCAA | 0.087 | 0.080 | 0.965 | 0.924 | 0.880 | 0.778 |

Each AADM consists of three ratios with a different branched chain amino acid (BCAA) in the denominator.
Abbreviations: Pos., positive, Pred., predictive; Train, training set; Test, test set; Hyp, 4-Hydroxyproline; Hse, Homoserine; Orn, Ornithine.
^{a}AADMs are a reproducible metabotype that is identified across training and test populations with a sensitivity greater than 5% and a positive predictive value greater than 90%.

AADMs Define a Diagnostic for BCAA Dysregulation Associated with ASD

The ASD subjects identified by each AADM were evaluated to assess the extent of overlap. We found that there is substantial overlap of the subjects identified by each of the metabotypes. However, each of the metabotypes also identifies a unique group of subjects. The AADM_{glutamine} identified 7.9% of the ASD subjects in the total CAMP population, AADM_{glycine} 9.7%, and the AADMₒᵣₙᵢₜₕᵢₙₑ 9.1%, with PPVs of 97.6%, 94.3% and 92.2% respectively. Combining all three AADM subtypes together (AADMₜₒₜₐₗ), identified 16.7% of ASD subjects in the CAMP population with a specificity of 96.3% and a PPV of 93.5%. Principal component analysis (PCA) of the metabolite ratios utilized in AADM_{glycine}, AADM_{glutamine}, and AADMₒᵣₙᵢₜₕᵢₙₑ was performed to test if an unsupervised method could identify subjects with AA dysregulation. A majority (80%, 74/92) of the AADM-positive subjects were separated from the unaffected subjects.

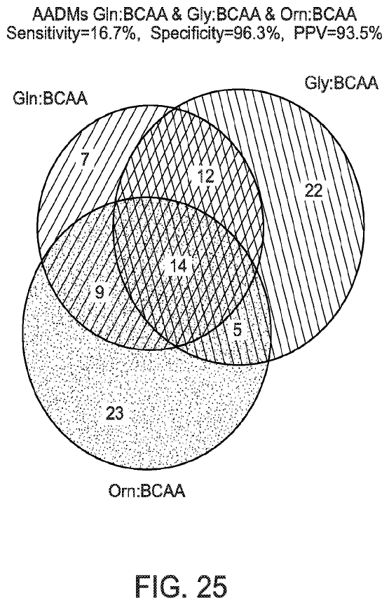

FIG. 25 shows a Venn diagram of the 92 AADMₜₒₜₐₗ subjects identified by each of the AADMs. At least 50% of the subjects identified by one AADM were identified by the other 2 AADMs. The AADMₜₒₜₐₗ population is composed of 86 ASD and 6 TYP subjects. The overall prevalence of metabolic dysregulation in the CAMP ASD population is 16.7% (86 AADMₜₒₜₐₗ ASD/516 CAMP ASD), specificity 96.3% (158 AADM-negative TYP/164 CAMP TYP), PPV 93.5% (86 AADMₜₒₜₐₗ ASD/92 AADMₜₒₜₐₗ).

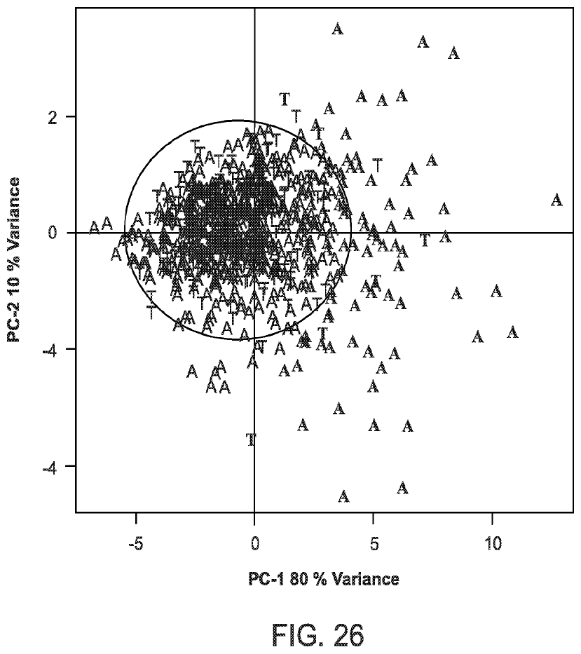

FIG. 26 is graph showing the principal comment analysis of the metabolite ratios used in the metabolic signature of the reproducible AADMs creating the AADMₜₒₜₐₗ estimates in the CAMP study population. Black circle is the 95% confidence interval from the Hotelling's T2. Red letters are AADMₜₒₜₐₗ positive (N=92), Black letters are AADMₜₒₜₐₗ negative (N=588). A=ASD and T=TYP.

AADM_{Ornithine} and AADM_{Glutamine} are More Sensitive at Detecting Females with ASD.

Since the composition of subject sex and age differed between the ASD and TYP populations, the impact of these variables was evaluated in the AADM positive and negative populations. Differential analysis of reproducible AADM positive and negative subjects' metabolite levels with respect to age or sex did not identify statistically significant changes in abundance. Differential analysis of age bins and correlation of age in the branched chain amino acid (BCAA) metabotype-positive population from the entire study population is shown in Table 13.

TABLE 13

| AADM | Metabolite | Pearson | Correlation | Correlation | ANOVA | Anova |
|---|---|---|---|---|---|---|
| Diagnostic | or Ratio | Correlation | p−value | FDR | p−value | FDR |
| Gln:BCAA | Gln:Ile | −0.122 | 0.443 | 0.588 | 0.889 | 0.984 |
| Gln:BCAA | Gln:Leu | −0.099 | 0.532 | 0.650 | 0.725 | 0.984 |
| Gln:BCAA | Gln:Val | 0.008 | 0.959 | 0.975 | 0.384 | 0.984 |
| Gln:BCAA | Gln | 0.200 | 0.205 | 0.454 | 0.771 | 0.984 |
| Gln:BCAA | Ile | 0.281 | 0.071 | 0.328 | 0.701 | 0.984 |
| Gln:BCAA | Leu | 0.273 | 0.080 | 0.328 | 0.552 | 0.984 |
| Gln:BCAA | Val | 0.191 | 0.226 | 0.483 | 0.628 | 0.984 |
| Gly:BCAA | Gly:Ile | −0.173 | 0.215 | 0.417 | 0.838 | 0.991 |
| Gly:BCAA | Gly:Leu | −0.128 | 0.359 | 0.550 | 0.659 | 0.991 |
| Gly:BCAA | Gly:Val | −0.076 | 0.591 | 0.731 | 0.249 | 0.991 |
| Gly:BCAA | Gly | −0.010 | 0.944 | 0.959 | 0.814 | 0.991 |
| Gly:BCAA | Ile | 0.180 | 0.198 | 0.396 | 0.967 | 0.999 |
| Gly:BCAA | Leu | 0.131 | 0.350 | 0.550 | 0.803 | 0.991 |
| Gly:BCAA | Val | 0.066 | 0.640 | 0.758 | 0.169 | 0.991 |
| Orn:BCAA | Ile | 0.180 | 0.198 | 0.396 | 0.967 | 0.999 |
| Orn:BCAA | Leu | 0.131 | 0.350 | 0.550 | 0.803 | 0.991 |
| Orn:BCAA | Orn:Leu | −0.053 | 0.712 | 0.761 | 0.746 | 0.761 |
| Orn:BCAA | Orn:Ile | 0.277 | 0.045 | 0.174 | 0.861 | 0.991 |
| Orn:BCAA | Orn:Val | 0.271 | 0.050 | 0.181 | 1.000 | 1.000 |
| Orn:BCAA | Orn | 0.340 | 0.013 | 0.127 | 0.289 | 0.991 |
| Orn:BCAA | Val | 0.066 | 0.640 | 0.758 | 0.169 | 0.991 |
| Total | Gln:Ile | −0.025 | 0.814 | 0.849 | 0.768 | 0.977 |
| Total | Gln:Leu | −0.024 | 0.821 | 0.849 | 0.772 | 0.977 |
| Total | Gln:Val | 0.027 | 0.796 | 0.849 | 0.164 | 0.977 |
| Total | Gln | 0.199 | 0.057 | 0.169 | 0.930 | 0.983 |
| Total | Gly:Ile | −0.266 | 0.011 | 0.081 | 0.739 | 0.977 |
| Total | Gly:Leu | −0.241 | 0.021 | 0.108 | 0.756 | 0.977 |
| Total | Gly:Val | −0.202 | 0.054 | 0.166 | 0.259 | 0.977 |
| Total | Gly | −0.147 | 0.162 | 0.310 | 0.882 | 0.977 |
| Total | Ile | 0.187 | 0.074 | 0.198 | 0.784 | 0.977 |
| Total | Leu | 0.191 | 0.068 | 0.193 | 0.770 | 0.977 |
| Total | Orn:Ile | 0.143 | 0.173 | 0.310 | 0.677 | 0.977 |
| Total | Orn:Leu | 0.143 | 0.175 | 0.310 | 0.683 | 0.977 |
| Total | Orn:Val | 0.205 | 0.050 | 0.165 | 0.596 | 0.977 |
| Total | Orn | 0.255 | 0.014 | 0.093 | 0.559 | 0.977 |
| Total | Val | 0.127 | 0.227 | 0.371 | 0.125 | 0.977 |

Analysis of variance of metabolite and ratio values was performed using the 6 month age bins (18-24,24-30,30-36,36-42,42-48) as the main effect. Pearson correlations are between the metabolite or ratio value and the subject's age in months.
Abbreviations: AADM, Amino Acid Dysregulation Metabotype; ANOVA, Analysis of variance; FDR, false discovery rate corrected ANOVA p-values.

### Significant at FDR<0.05.

Differential analysis of the metabolite and ratio abundance values comparing Amino Acid Dysregulation Metabotypes (AADMtotal)-positive and AADMtotal-negative populations from the entire study population is shown in Table 14.

TABLE 14

| Metabolite | Fold | | Count | | |
|---|---|---|---|---|---|
| or | Change | Welch | Nega- | Count | |
| Ratio | (POS/NEG) | p-value | tive | Positive | FDR |
| Gln | 1.159 | 7.16996E−16 | 588 | 92 | 8.23E−16 |
| Gly | 1.381 | 4.14595E−25 | 588 | 92 | 6.12E−25 |
| Ile | 0.771 | 1.03268E−23 | 588 | 92 | 1.46E−23 |
| Leu | 0.739 | 9.40425E−30 | 588 | 92 | 1.62E−29 |
| Orn | 1.351 | 6.88894E−17 | 588 | 92 | 8.21E−17 |
| Val | 0.76 | 8.21497E−26 | 588 | 92 | 1.27E−25 |
| Gln:Ile | 1.467 | 7.2817E−36 | 588 | 92 | 2.05E−35 |
| Gln:Leu | 1.542 | 2.81544E−37 | 588 | 92 | 9.7E−37 |
| Gln:Val | 1.496 | 2.12406E−37 | 588 | 92 | 8.23E−37 |
| Gly:Ile | 1.728 | 8.1802E−44 | 588 | 92 | 1.27E−42 |
| Gly:Leu | 1.82 | 3.95539E−42 | 588 | 92 | 3.07E−41 |
| Gly:Val | 1.775 | 5.00064E−40 | 588 | 92 | 3.1E−39 |
| Orn:Ile | 1.7 | 2.01991E−39 | 588 | 92 | 1.04E−38 |
| Orn:Leu | 1.78 | 7.70795E−43 | 588 | 92 | 7.96E−42 |
| Orn:Val | 1.727 | 1.06689E−45 | 588 | 92 | 3.31E−44 |

### Significant at FDR<0.05.

Abbreviations: NEG, AADM-Negative; POS, AADM-positive; FDR, false discovery rate corrected p-value from Welch T-Tests.
Females with ASD were 2.1 fold (odds ratio 2.8, p value 0.002) more likely to be identified by AADMₒᵣₙᵢₜₕᵢₙₑ and AADM_{glutamine} than would be expected by chance.
Fisher exact test for gender bias in each panel and across reproducible Amino Acid Dysregulation Metabotypes (AADMs) is shown in Table 15.

TABLE 15

| | Odds | p- | Exp | Obs |
|---|---|---|---|---|
| AADM | Ratio | value | Freq | Freq |
| AADM_{Glutamine} | 2.902 | 0.002 | 22% | 41% |
| AADM_{Glycine} | 1.498 | 0.274 | 22% | 28% |
| AADM_{Ornithine} | 2.812 | 0.002 | 22% | 40% |
| AADM_{Total} | 2.339 | 0.001 | 23% | 35% |

“Exp Freq” is the expected frequency of females in the metabotype and “Obs Freq” is the observed frequency of females in the metabotype. AADMₜₒₜₐₗ indicates all subjects identified by one or more of the metabotypes, AADMₒᵣₙᵢₜₕᵢₙₑ, AADM_{glutamine} or AADM_{glycine}. Abbreviations: AADM, amino acid dysregulation metabotype.
The AADM_{glycine} did not demonstrate a predictive sex bias.

### Example 4: Discussion

CAMP is the largest study of the metabolism of children with autism spectrum disorder and age-matched typically developing children carried out to date. Metabolomics offers the opportunity to examine associations between small molecule abundance levels and the presence of a disorder such as ASD as well as influences such as sex, severity of the disorder, comorbid conditions, diet, supplements and other environmental factors. Given the known heterogeneity of ASD, the size of CAMP offers the prospect of identifying metabolically defined subtypes (or metabotypes) that can identify groups with a prevalence as low as 5%. Diagnostic tests for metabotypes of ASD create an opportunity for earlier diagnosis and the potential to inform more targeted treatment.

Our goal is to analyze data from the CAMP population to identify metabotypes associated with ASD that could enable stratification of the disorder based on shared metabolic characteristics. Based on our own observations and growing literature (23-25, 31, 32) reporting a dysregulation of amino acid metabolism associated with ASD, we began our analysis by studying free plasma amine levels. A simple analysis of the mean concentrations of free plasma amines did not reveal meaningful differences between the ASD and TYP populations of children. However, scatterplots of amine levels indicated that there were subsets of children with ASD with amine levels at the extreme upper or lower end of the abundance distribution. Moreover, correlation analyses revealed two negatively correlated clusters of related metabolites. We tested if ratios of these metabolites could identify subpopulations that exhibit dysregulation of AA metabolism associated with ASD. Diagnostic thresholds established in the training set of subjects using ratios of glutamine, glycine, ornithine and serine with leucine, isoleucine and valine (BCAAs) reproducibly detected subpopulations in an independent test set. Three AADMs based on an imbalance of glutamine, glycine, or ornithine with the BCAAs were reproduced across training and test sets of subjects. Separately, each AADM identified ASD subjects with 7-10% sensitivity and 92-98% PPVs. Taken together, all AADMs identified an altered metabolic phenotype of imbalanced BCAA metabolism in 16.7% of CAMP ASD subjects with a specificity of 96.3% and PPV of 93.5%.

Identification of ASD children with altered AADMs represents an important step toward understanding the etiology of one form of ASD. Imbalances in BCAAs in plasma have been shown to alter not only brain levels of BCAAs, but also other amino acids important for key metabolic processes including intermediary metabolism, protein synthesis, and neurotransmission. For example, when plasma BCAA levels are reduced due to a rare genetic defect in branched chain ketoacid dehydrogenase kinase (BCKDK) (24) leading to accelerated BCAA degradation, the transporters that are normally responsible for their import into the brain transport an excess of other amino acids instead. And, this condition is associated with ASD (24). Similarly, Tarlungeanu (25) demonstrated that rare disruption of amino acid transport associated with defects in the LAT1 transporter reduced the uptake of BCAAs into the brain; again this was associated with ASD-like symptoms. Interestingly, neither study reported elevated plasma levels of glycine, ornithine, or glutamine. The imbalance of amino acid levels in CAMP strongly suggests that other perturbations in BCAA metabolism may be a risk factor for the development of ASD. Importantly, the metabolomic results reported here provide a mechanism for stratifying the larger group of children with ASD into an AADM positive subgroup to enable a more targeted approach to understanding the etiology of this form of ASD. For example, the AADMs we identified may reveal a disruption of the mTORC1 system which could be an underlying reason for lower free plasma BCAA levels. Cellular levels of BCAA as well as other amino acids are maintained through signaling associated mTORC1 and the transcription factor ATF4 (33). Dysregulation of the mTOR pathway is an underlying cause of amino acid dysregulation that is associated with ASD and tuberous sclerosis (34).

The AADMs provide one pathway to much earlier diagnosis of a substantial subset of children with ASD. Earlier diagnosis may also provide the opportunity for earlier biological intervention. BCAA supplementation or high protein diet has been used in mouse models (24) and human patients (31) with BCKDK deficiency to successfully reduce ASD symptoms and improve cognitive function. Defining a group of AADM positive children may enable stratification of the autistic population as a precursor to targeted intervention through dietary supplementation or specialized diet. Currently, clinical trials of common therapies such as vitamin and mineral supplements, carnitine and gluten-free casein-free diets, apply these therapies to all participants. Metabotyping subjects prior to treatment and monitoring metabolite levels provides the opportunity to assess patient compliance and response, and to make adjustments to treatment based on objective measurement of the metabolic profile of the individual subject. It is likely that this strategy would substantially improve positive treatment outcomes.

This study does have some limitations. The levels of blood plasma amine metabolites are not directly relatable to brain levels (35) making direct association of changes in plasma levels to changes in brain levels difficult. The CAMP study focused on recruitment of a large sample of children with ASD and age-matched typically developing controls. Logistical and financial constraints precluded our ability to recruit a large enough sample of children with developmental delays without ASD. Thus, the specificity of ADDM for ASD relative to other neurodevelopmental disorders is currently unclear. This is an important issue that will need to be resolved in future studies. In addition, longitudinal samples are not available to analyze whether AADMs are stable over time. Finally, this study lacks animal models or tissue samples that could be used to dissect enzymatic and expression analysis to identify the molecular mechanisms underlying AADM. While we cannot explain the alterations in metabolism, we have demonstrated that our approach provides stratification of subjects for which future studies and perhaps targeted treatments could be carried out.

This study demonstrates one approach to analyzing the metabolism of ASD to successfully identify reproducible metabotypes. Analysis of the CAMP study samples is ongoing and there will be additional metabotypes which will be diagnostic for subsets of children with ASD. Stratifying ASD based on metabotypes offers an opportunity to identify efficacious interventions within metabotypes that can lead to more precise and individualized treatment. The hope is that by combining the established metabotypes into a more comprehensive diagnostic system, that a substantial percentage of children at risk for ASD will be identifiable at a very early age.

### Example 5: Supplemental Methods

### Mass Spectrometry

Mass spectroscopy (MS) was performed using electrospray ionization in positive ion mode with an Agilent QqQ 6490 triple quadrupole mass spectrometer. Analyte selectivity used a combination of product/precursor mass transitions and retention time. Agilent MassHunter Quantitative Analysis software (version B.06.00) was used for quantitation of liquid chromatography (LC) MS/MS data. Dynamic Multiple Reaction Monitoring (MRM) was utilized to assign optimal dwell times for each analyte.

Stable isotope labeled (SIL) internal standards were used to normalize the signal for each analyte to account for variations in the matrix and sample preparation. For analytes in which no SIL internal standard was available, a surrogate SIL internal standard was chosen based on the work of Gray et al. (1) using a structurally similar analyte.

Chromatographic separation was performed using reverse-phase chromatography on a HSS T3 2.1×150 mm, 1.8 μm column (Waters). Column temperature was maintained at 45° C. The mobile phase was composed of 0.1% formic acid in water and 0.1% formic acid in acetonitrile. A gradient elution was performed which separates the analytes over the course of 7.5 minutes per injection using a flow rate of 0.6 ml/min.

Samples were evaluated relative to calibration standards measured in each analysis batch. Samples that measured below the lowest concentration level of the calibration standard were reported as having a concentration of 0.00 μM. Samples with an analyte(s) that quantified above the highest concentration level calibration standard were diluted and reanalyzed to obtain a measurement within the range of valid quantification for that analyte.

### Example 7: Supplemental Results

Abundance of Metabolite Ratios and Metabolites Used in the Ratios are not Changed in AADM Positive and AADM Negative Subjects with Respect to Age and Sex

The mean levels of metabolite ratios or metabolites in the AADM positive and ADDM negative populations were not different (FDR>0.05) in males and females indicating that the sex bias in detection of the AADM_{glutamine}, AADMₒᵣₙᵢₜₕᵢₙₑ and AADMₜₒₜₐₗ positive populations is not evident in the levels of metabolites within AADM positive and negative populations. Differential analysis of subject sex in the Amino Acid Dysregulation Metabotypes (AADM)-positive population from the entire study population is shown in Table 16.

TABLE 16

| AADM | Metabolite | Fold Change | Welch | Count | Count | Welch |
|---|---|---|---|---|---|---|
| Diagnostic | or Ratio | (Female/Male) | p-value | Male | Female | FDR |
| Gln:BCAA | Gln:Ile | 0.992 | 0.70936 | 25 | 17 | 0.7854 |
| Gln:BCAA | Gln:Leu | 0.965 | 0.46069 | 25 | 17 | 0.7757 |
| Gln:BCAA | Gln:Val | 1.012 | 0.6506 | 25 | 17 | 0.7757 |
| Gln:BCAA | Gln | 1.034 | 0.37278 | 25 | 17 | 0.7756 |
| Gln:BCAA | Ile | 1.056 | 0.27118 | 25 | 17 | 0.6908 |
| Gln:BCAA | Leu | 1.067 | 0.13127 | 25 | 17 | 0.5813 |
| Gln:BCAA | Val | 1.023 | 0.64978 | 25 | 17 | 0.7757 |
| Gly:BCAA | Gly:Ile | 0.983 | 0.77244 | 38 | 15 | 0.9861 |
| Gly:BCAA | Gly:Leu | 0.996 | 0.86762 | 38 | 15 | 0.9861 |
| Gly:BCAA | Gly:Val | 1.077 | 0.09774 | 38 | 15 | 0.4661 |
| Gly:BCAA | Gly | 0.997 | 0.9539 | 38 | 15 | 0.9936 |
| Gly:BCAA | Ile | 1.007 | 0.653 | 38 | 15 | 0.9201 |
| Gly:BCAA | Leu | 0.983 | 0.89071 | 38 | 15 | 0.9861 |
| Gly:BCAA | Val | 0.918 | 0.05788 | 38 | 15 | 0.3394 |
| Orn:BCAA | Ile | 1.114 | 0.07955 | 30 | 21 | 0.7194 |
| Orn:BCAA | Leu | 1.08 | 0.14541 | 30 | 21 | 0.7194 |
| Orn:BCAA | Orn:Ile | 1.009 | 0.8684 | 30 | 21 | 0.9446 |
| Orn:BCAA | Orn:Leu | 1.025 | 0.5839 | 30 | 21 | 0.8255 |
| Orn:BCAA | Orn:Val | 1.054 | 0.15999 | 30 | 21 | 0.7194 |
| Orn:BCAA | Orn | 1.127 | 0.06133 | 30 | 21 | 0.7194 |
| Orn:BCAA | Va | 1.063 | 0.36272 | 30 | 21 | 0.7755 |
| Total | Gln:Ile | 1.033 | 0.53384 | 59 | 33 | 0.8073 |
| Total | Gln:Leu | 1.026 | 0.4939 | 59 | 33 | 0.7852 |
| Total | Gln:Val | 1.056 | 0.23321 | 59 | 33 | 0.6702 |
| Total | Gln | 1.048 | 0.09376 | 59 | 33 | 0.4994 |
| Total | Gly:Ile | 0.918 | 0.13093 | 59 | 33 | 0.5018 |
| Total | Gly:Leu | 0.953 | 0.27204 | 59 | 33 | 0.6993 |
| Total | Gly:Val | 1.031 | 0.60514 | 59 | 33 | 0.8453 |
| Total | Gly | 0.926 | 0.1138 | 59 | 33 | 0.5018 |
| Total | Ile | 1.031 | 0.60514 | 59 | 33 | 0.8453 |
| Total | Leu | 1.02 | 0.63138 | 59 | 33 | 0.8453 |
| Total | Orn:Ile | 1.081 | 0.15733 | 59 | 33 | 0.5134 |
| Total | Orn:Leu | 1.082 | 0.13759 | 59 | 33 | 0.5018 |
| Total | Orn:Val | 1.106 | 0.03391 | 59 | 33 | 0.3804 |
| Total | Orn | 1.126 | 0.11793 | 59 | 33 | 0.5018 |
| Total | Val | 1.003 | 0.91603 | 59 | 33 | 0.931 |

T-tests were used to test for differences in mean abundance of male and female populations. Abbreviations: AADM, Amino Acid Dysregulation Metabotype; Welch, Welch T-test; FDR, false discovery rate corrected p-value from Welch T-Tests.

### Significant at FDR<0.05.

Since there are slight differences between the age of TYP and ASD subjects (3.3 months) and between the age of ASD subjects in the training and test set (1.4 months), we tested if the age of the subject within AADM positive population was associated with the ratio of metabolite abundance levels. No differences in mean (FDR>0.05) or correlations (FDR>0.05) of abundance levels of metabolite ratios or individual metabolites of AADM positive populations were found in association with the age of the subjects.

### Metabolite Ratios and Metabolites Used in the Ratios are Differentially Abundant in the AADM Total Positive Population

Differential analysis of the AADMₜₒₜₐₗ positive and negative populations was performed to test if differences in the metabolites are present. The mean levels of metabolite ratios used to identify the AADMₜₒₜₐₗ population were increased by 47-82% (FDR<0.001) in the AADMₜₒₜₐₗ positive population when compared to the AADMₜₒₜₐₗ negative population. The mean levels of numerator metabolites glutamine, glycine, and ornithine were increased by 16-38% (FDR<0.001) and BCAA metabolites were decreased by 23-26% (FDR<0.001) in the AADMₜₒₜₐₗ positive population compared to the AADMₜₒₜₐₗ negative population.

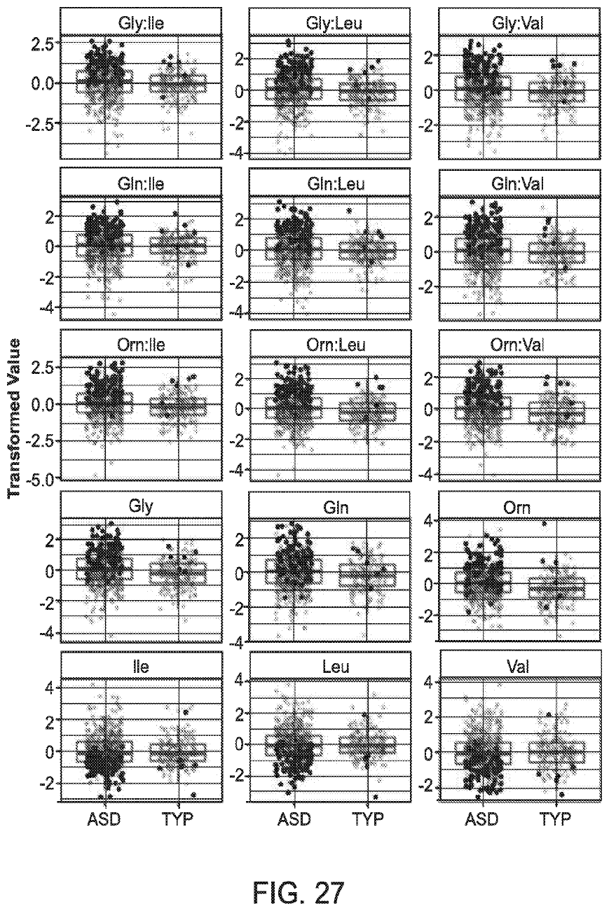

FIG. 27 shows scatter plots of the ratios of levels of metabolites and levels of individual metabolites utilized in identification of AADMs. Red points are AADMₜₒₜₐₗ positive subjects, and black points are AADMₜₒₜₐₗ negative subjects.

### Example 6: Sample Test Report

A sample test report is provided below.

### Patient and Sample Data

The following information about the patient and sample is provided: patient's name, patient's date of birth, patient's sex, specimen type (e.g., plasma), date of specimen collection, date specimen was received, test panel used, and date and time of test report.

### Results Summary

The algorithmic analysis indicates patient has form(s) of amino acid dysregulation associated with autism spectrum disorder (ASD). Specific details of findings are listed below.

Metabotype 8: An imbalance between the plasma concentrations of Ornithine and Phenylalanine was detected. This imbalance includes above average Ornithine.

Metabotype 12: An imbalance between the plasma concentrations of Ornithine and branched chain amino acids (BCAA) was detected. This imbalance includes above average Ornithine and below average BCAA.

Metabotype 15: An imbalance between the plasma concentrations of Ornithine and Kynurenine was detected. This imbalance generally indicates plasma concentrations of Ornithine which are above average and kynurenine which is below average.

Metabotype 16: An imbalance between the plasma concentrations of Ornithine and Lysine was detected. This imbalance generally indicates plasma concentrations of Ornithine which are above average.

Additional Findings: Levels of individual analytes tested are all within the normal range. The following analytes were tested: Alanine, Arginine, Asparagine, Aspartic Acid, ß-Alanine, ß-Aminoisobutyric Acid, Citrulline, Ethanolamine, β-Aminoisobutyric Acid, Glutamic Acid, Glutamine, Glycine, Histidine, Homocitrulline, Homoserine, Isoleucine, Kynurenine, Leucine, Lysine, Methionine, Ornithine, Phenylalanine, Proline, Sarcosine, Serine, Serotonin, Taurine, Threonine, Tryptophan, Tyrosine, Valine, and 4-Hydroxyproline. Levels of individual analytes are provided in Table 17.

TABLE 17
Amine Results Table: Individual measurements
of analytes by LC-MS/MS.

| | | Normal | | |
|---|---|---|---|---|
| # | Analyte | Range (μM) | Result (μM) | Flag |
| 1 | Alanine | 144-423 | 238 | Normal |
| 2 | Arginine | 44-100 | 42 | Low |
| 3 | Asparagine | 25-56 | 43 | Normal |
| 4 | Aspartic Acid | 1.5-4.9 | 1.8 | Normal |
| 5 | Beta-Alanine | 1.8-7.8 | 1.6 | Low |
| 6 | Beta-Aminoisobutyric Acid | 0.5-5 | 1.8 | Normal |
| 7 | Citrulline | 16-39 | 29 | Normal |
| 8 | Ethanolamine | 5-10 | 5.3 | Normal |
| 9 | Gamma-aminobutyric Acid | 0.18-0.42 | 0.45 | High |
| 10 | Glutamic Acid | 17-92 | 20 | Normal |
| 11 | Glutamine | 385-646 | 489 | Normal |
| 12 | Glycine | 137-334 | 241 | Normal |
| 13 | Histidine | 56-99 | 70 | Normal |
| 14 | Homocitrulline | 0.14-0.51 | 0.33 | Normal |
| 15 | Homoserine | 0.1-0.18 | 0.15 | Normal |
| 16 | Isoleucine | 35-83 | 43 | Normal |
| 17 | Kynurenine | 1.2-3.2 | 1.4 | Normal |
| 18 | Leucine | 67-138 | 86 | Normal |
| 19 | Lysine | 76-174 | 129 | Normal |
| 20 | Methionine | 12-30 | 20 | Normal |
| 21 | Ornithine | 20-54 | 80 | High |
| 22 | Phenylalanine | 37-64 | 35 | Low |
| 23 | Proline | 77-231 | 117 | Normal |
| 24 | Sarcosine | 0.7-2.2 | 5.9 | High |
| 25 | Serine | 70-137 | 140 | High |
| 26 | Serotonin | 0.04-0.56 | 0.08 | Normal |
| 27 | Taurine | 24-72 | 55 | Normal |
| 28 | Threonine | 51-157 | 81 | Normal |
| 29 | Tryptophan | 31-93 | 37 | Normal |
| 30 | Tyrosine | 38-100 | 41 | Normal |
| 31 | Valine | 138-323 | 164 | Normal |
| 32 | 4-Hydroxyproline | 12-41 | 29 | Normal |

Reference values are the 2.5-97.5 percentiles obtained from specially developing children (18-48 months old) in the CAMP-Q1 study.

### Exemplary Guidance

Recommend follow up with neurodevelopment/ASD specialist for further evaluation. Some studies indicate dietary modification may be beneficial for patients with metabolic dysregulation. May want to refer patient to a registered dietitian nutritionist (RDN) for an evaluation of his/her diet and supplement intake.

### Example 7: Results from Metabolomic Studies

110 subjects from the Children's Autism Metabolome Project (CAMP) study were sent to Quest for amino acid analysis.

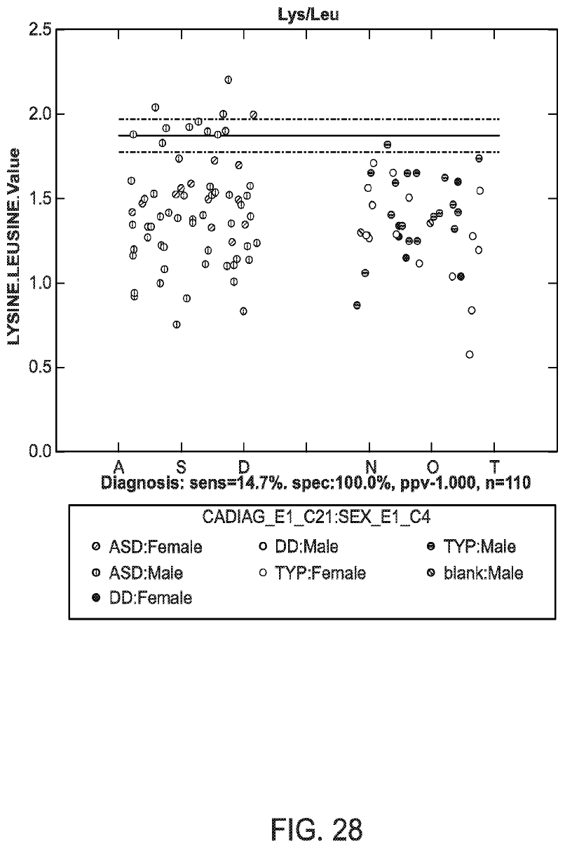

FIG. 28 is a graph of ratios of concentrations of lysine to leucine obtained from the Quest analysis of subjects from the CAMP study. Subjects were from the following classes: red circles, females with autism spectrum disorders (ASD); green circles, males with autism spectrum disorders; purple circles, females with developmental delay (DD); light blue circles, males with developmental delay; yellow circles, typically developing females; dark blue circles, typically developing males; a grey circles, male blanks. Ratios from subjects having autism spectrum disorders are shown on the left side of the graph, and ratios from subjects having developmental delay and from typically developing subjects are shown in the right side of the graph.

272 subjects from the CAMP study were analyzed using the NeuroPoint diagnostic panel. Of those subject, results from the 195 male subjects were selected as most revealing. Subjects were placed in two categories: typically developing (TYP), no indication of ASD or DD; and not typically developing (NOT), having either ASD or DD.

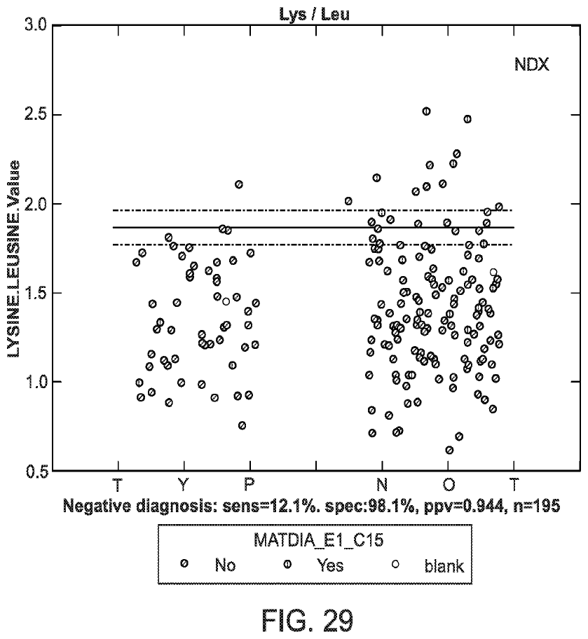

FIG. 29 is a graph of ratios of concentrations of lysine to leucine obtained from the NeuroPoint diagnostic analysis of the 195 male subjects from the CAMP study. Subjects were from the following classes: red circles, no MATDIA_E1_C15; green circles, MATDIA_E1_C15; and yellow circles, blank. Ratios from typically developing (TYP) subjects are shown on the left side of the graph, and ratios from not-typically developing (NOT) subject are shown on the right side of the graph.

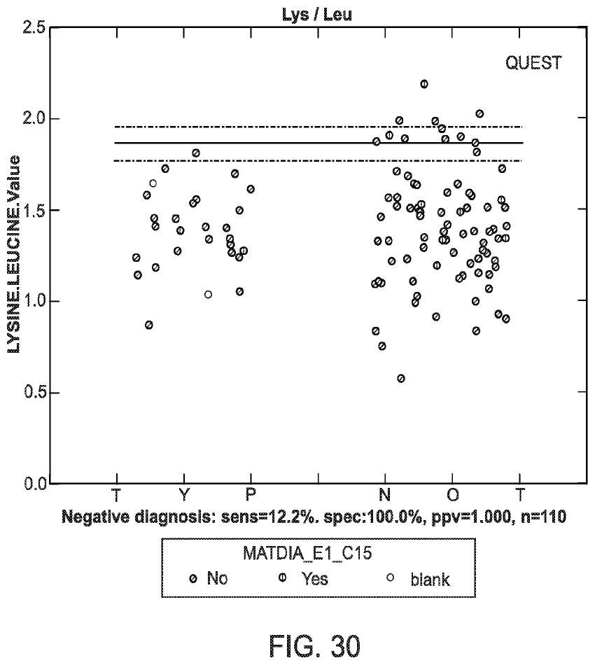

FIG. 30 is a graph of ratios of concentrations of lysine to leucine obtained from the Quest diagnostic analysis of the 195 male subjects from the CAMP study. Subjects were from the following classes: red circles, no MATDIA_E1_C15; green circles, MATDIA_E1_C15; and yellow circles, blank. Ratios from typically developing (TYP) subjects are shown on the left side of the graph, and ratios from not-typically developing (NOT) subject are shown on the right side of the graph.

Subjects from the CAMP study were analyzed by the NeuroPoint diagnostic analysis for ratios of concentrations of lysine to valine and lysine to isoleucine.

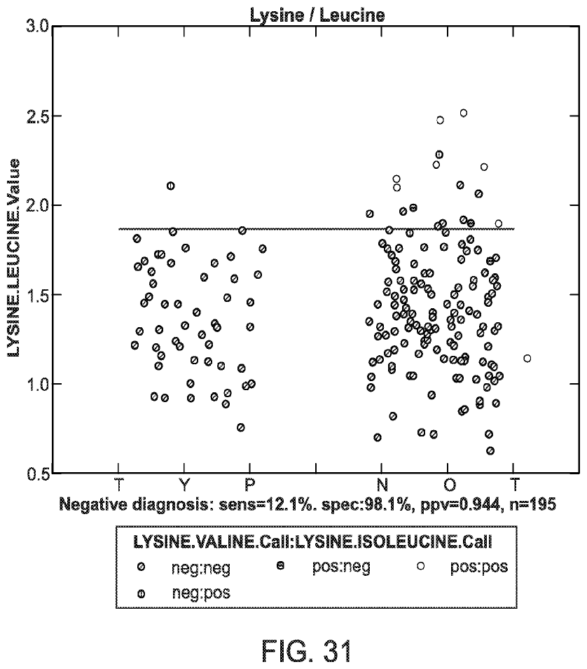

FIG. 31 is a graph of ratios of concentrations of lysine to leucine obtained from the NeuroPoint diagnostic analysis of the 195 male subjects from the CAMP study. Subjects were from the following classes: red circles, negative for both lysine:valine and lysine:isoleucine; green circles, negative for lysine:valine and positive for lysine:isoleucine; blue circle, positive for lysine:valine and negative for lysine:isoleucine; and yellow circles, positive for both lysine:valine and lysine:isoleucine. Ratios from typically developing (TYP) subjects are shown on the left side of the graph, and ratios from not-typically developing (NOT) subject are shown on the right side of the graph.

CAMP study subjects were analyzed to determine whether the branched chain amino acid (BCAA) subtype was associated with diet, medications, or supplements. Results for the analysis are provided in Table 18.

TABLE 18

| | | Odds | p- |
|---|---|---|---|
| Study Variable | Definition | ratio | value |
| MedSupOngoing_PHYSICAL_THERAPY_Yes | MedSupOngoing_PHYSICAL_THERAPY | 8.45 | <0.05 |
| MedSup_PHYSICAL_THERAPY_Yes | MedSup_PHYSICAL_THERAPY | 4.962 | <0.05 |
| DSMVYN_E1_C21_No | DSMVYN_E1_C21 | 0.321 | <0.05 |
| DSMVCOM_E1_C21_No | DSMVCOM_E1_C21 | 0.225 | <0.05 |
| MATDIA_E1_C15_Yes | MATDIA_E1_C15 | 3.397 | <0.05 |

Pathways for catabolism of branched chain amino acids are shown below:

BCAAs are important in neurological disorders. For example, defects in branched chain ketoacid dehydrogenase (BCKDH) lead to elevated plasma levels of BCAAs in maple syrup urine disease. In contrast, plasma levels of BCAAs are decreased due to deficiencies in BCKDH-kinase in rare families based on genetics with ASD, DD, and epilepsy. In such families, dietary supplementation with high levels of proteins restores plasma levels of BCAAs and improves Vineland scores.

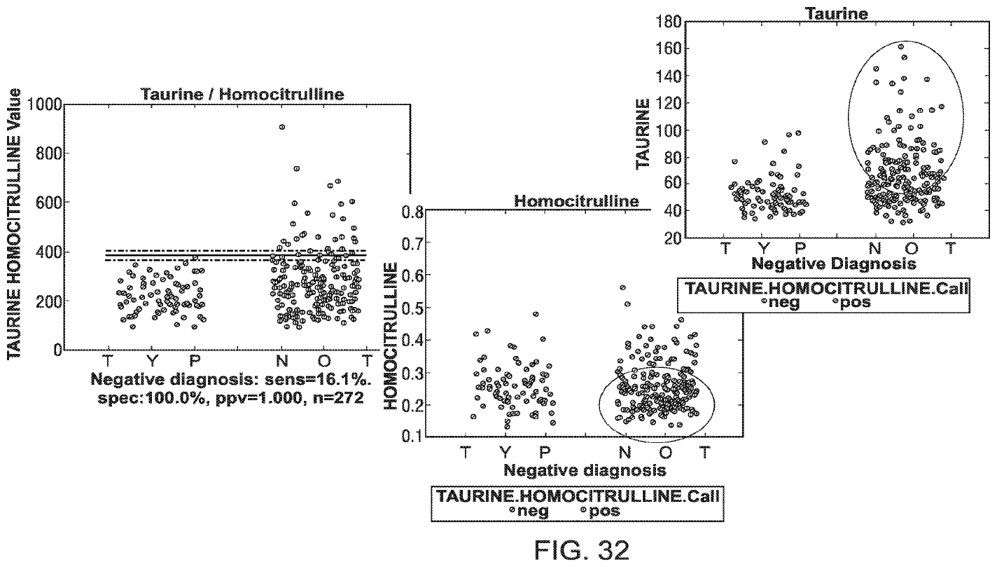

FIG. 32 is a series of graphs showing of concentrations of taurine and homocitrulline and ratios of concentrations of taurine to homocitrulline obtained from the NeuroPoint diagnostic analysis of the 195 male subjects from the CAMP study. Ratios of concentrations of taurine to homocitrulline are shown in graph on left, concentrations of homocitrulline are shown in graph in center, and concentrations of taurine are shown in graph on right. Subjects were from the following classes: red circles, negative diagnosis; green circles, positive diagnosis. Values from typically developing (TYP) subjects are shown on the left side of the graphs, and values from not-typically developing (NOT) subject are shown on the right side of the graphs.

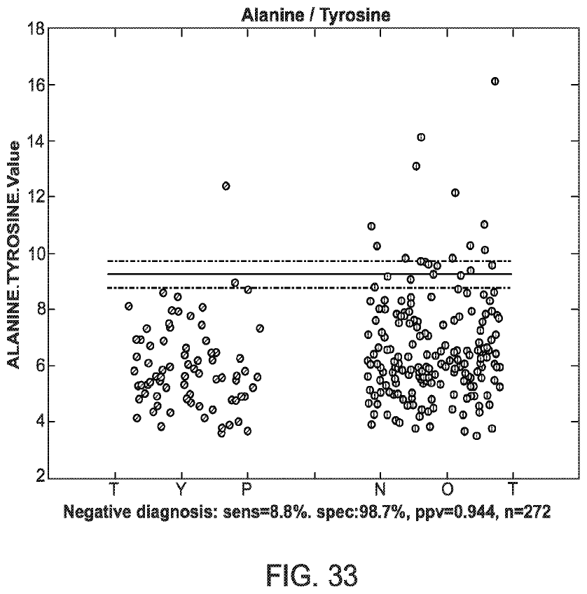

FIG. 33 is a graph showing ratios of concentrations of alanine to tyrosine obtained from the NeuroPoint diagnostic analysis of the 195 male subjects from the CAMP study. Subjects were from the following classes: red circles, negative diagnosis; green circles, positive diagnosis. Ratios from typically developing (TYP) subjects are shown on the left side of the graph, and ratios from not-typically developing (NOT) subject are shown on the right side of the graph.

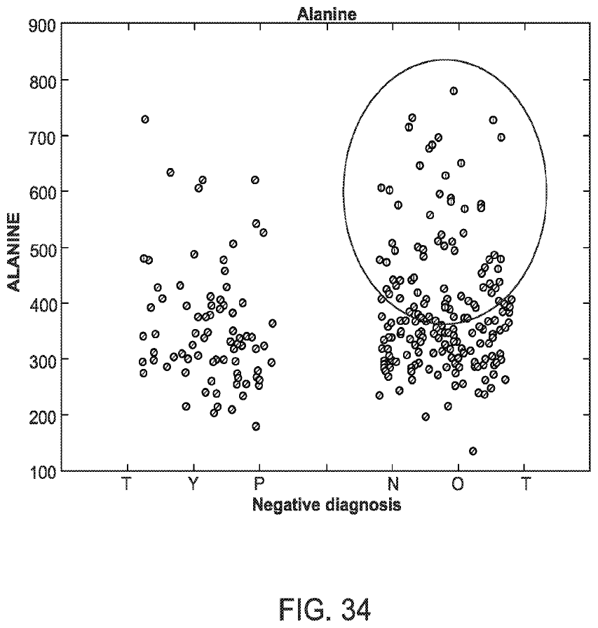

FIG. 34 is a graph showing concentrations of alanine obtained from the NeuroPoint diagnostic analysis of the 195 male subjects from the CAMP study. Subjects were from the following classes: red circles, negative diagnosis; green circles, positive diagnosis. Values from typically developing (TYP) subjects are shown on the left side of the graph, and values from not-typically developing (NOT) subject are shown on the right side of the graph.

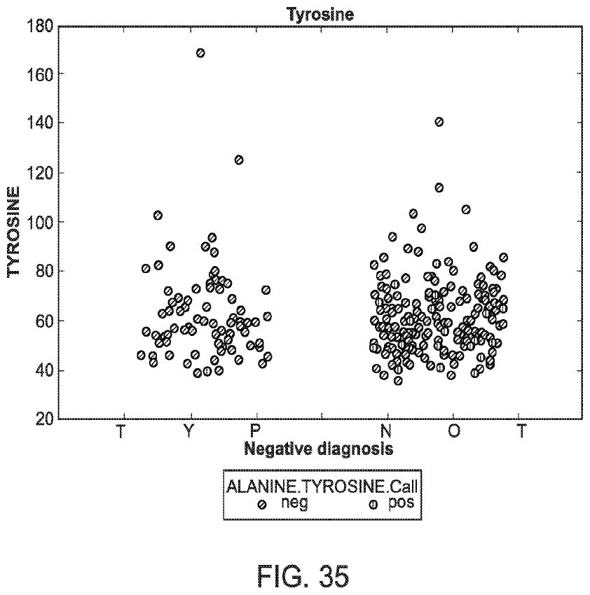

FIG. 35 is a graph showing concentrations of tyrosine obtained from the NeuroPoint diagnostic analysis of the 195 male subjects from the CAMP study. Subjects were from the following classes: red circles, negative diagnosis; green circles, positive diagnosis. Values from typically developing (TYP) subjects are shown on the left side of the graph, and values from not-typically developing (NOT) subject are shown on the right side of the graph.

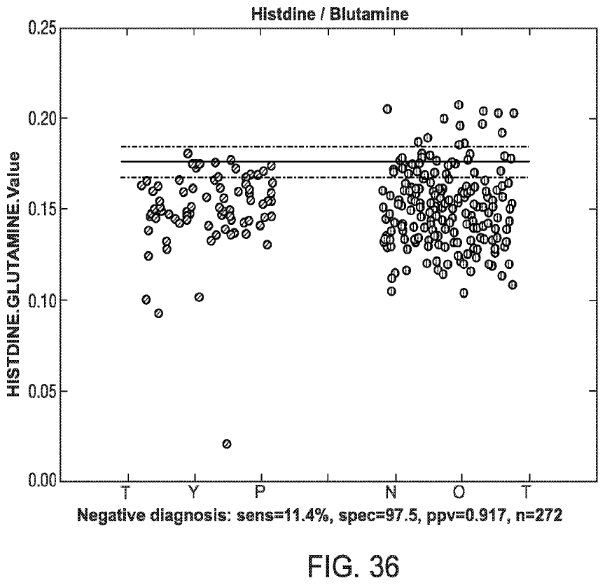

FIG. 36 is a graph showing ratios of concentrations of histidine to glutamine obtained from the NeuroPoint diagnostic analysis of the 195 male subjects from the CAMP study. Subjects were from the following classes: red circles, negative diagnosis; green circles, positive diagnosis. Ratios from typically developing (TYP) subjects are shown on the left side of the graph, and ratios from not-typically developing (NOT) subject are shown on the right side of the graph.

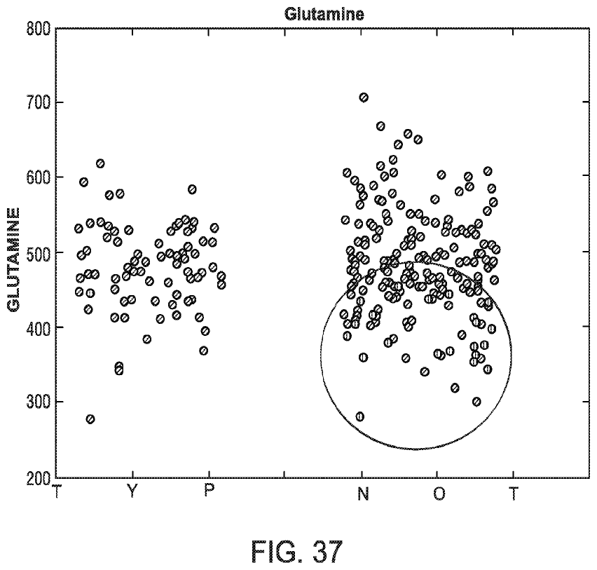

FIG. 37 is a graph showing concentrations of glutamine obtained from the NeuroPoint diagnostic analysis of the 195 male subjects from the CAMP study. Subjects were from the following classes: red circles, negative diagnosis; green circles, positive diagnosis. Values from typically developing (TYP) subjects are shown on the left side of the graph, and values from not-typically developing (NOT) subject are shown on the right side of the graph.

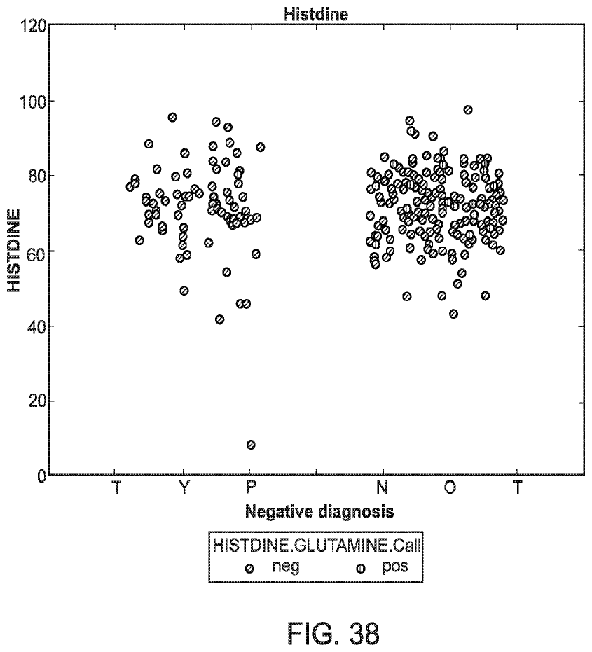

FIG. 38 is a graph showing concentrations of histidine obtained from the NeuroPoint diagnostic analysis of the 195 male subjects from the CAMP study. Subjects were from the following classes: red circles, negative diagnosis; green circles, positive diagnosis. Values from typically developing (TYP) subjects are shown on the left side of the graph, and values from not-typically developing (NOT) subject are shown on the right side of the graph.

CAMP study subjects were analyzed to determine whether ratios of concentrations of taurine to homocitrulline were associated with various variable. Results for the analysis are provided in Table 19.

TABLE 19

| Variable | P-value |
|---|---|
| Diagnosis-related variables | <0.001 |
| Breast fed | <0.01 |
| Occupational therapy | <0.05 |
| Melatonin supplement | <0.05 |
| Multivitamin supplement | <0.05 |
| Physical therapy | <0.05 |

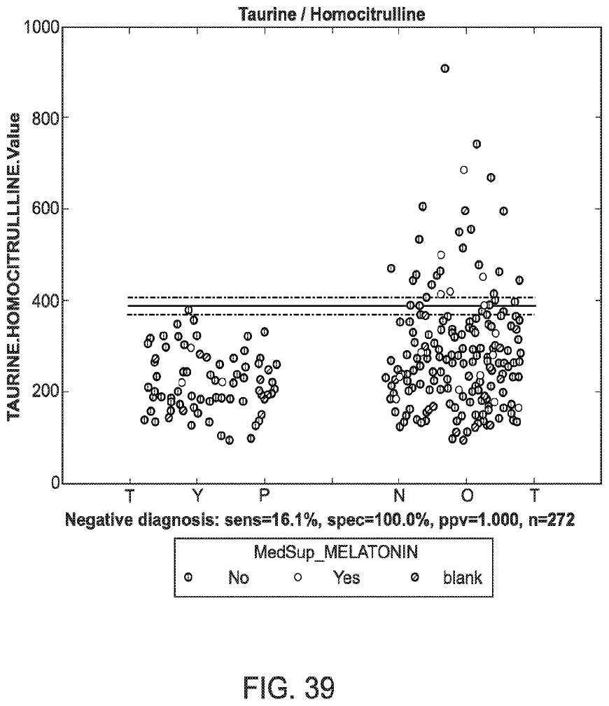

FIG. 39 is a graph showing ratios of concentrations of taurine to homocitrulline obtained from the NeuroPoint diagnostic analysis of the 195 male subjects from the CAMP study. Subjects were from the following classes: green circles, no melatonin supplementation; yellow circles, melatonin supplementation; and red circles, blank. Values from typically developing (TYP) subjects are shown on the left side of the graph, and values from not-typically developing (NOT) subject are shown on the right side of the graph.

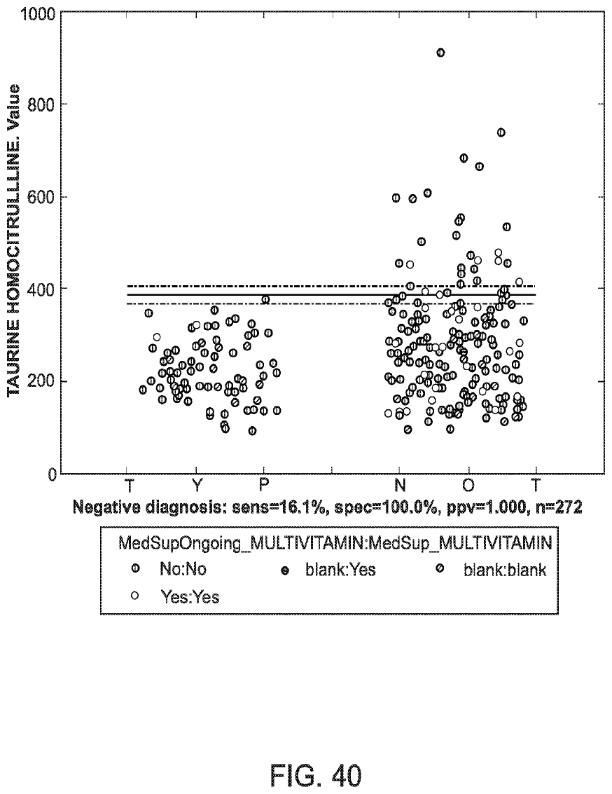

FIG. 40 is a graph showing ratios of concentrations of taurine to homocitrulline obtained from the NeuroPoint diagnostic analysis of the 195 male subjects from the CAMP study. Subjects were from the following classes: green circles, no multivitamin supplementation; yellow circles, ongoing multivitamin supplementation; blue circles, multivitamin supplementation; red circles, blank. Values from typically developing (TYP) subjects are shown on the left side of the graph, and values from not-typically developing (NOT) subject are shown on the right side of the graph.

CAMP study subjects were analyzed to determine whether ratios of concentrations of alanine to tyrosine were associated with various variable. Results for the analysis are provided in Table 20.

TABLE 20

| Variable | P-value |
|---|---|
| Diagnosis-related variables | <0.001 |
| High salt diet | <0.01 |
| Food preference for texture | <0.05 |

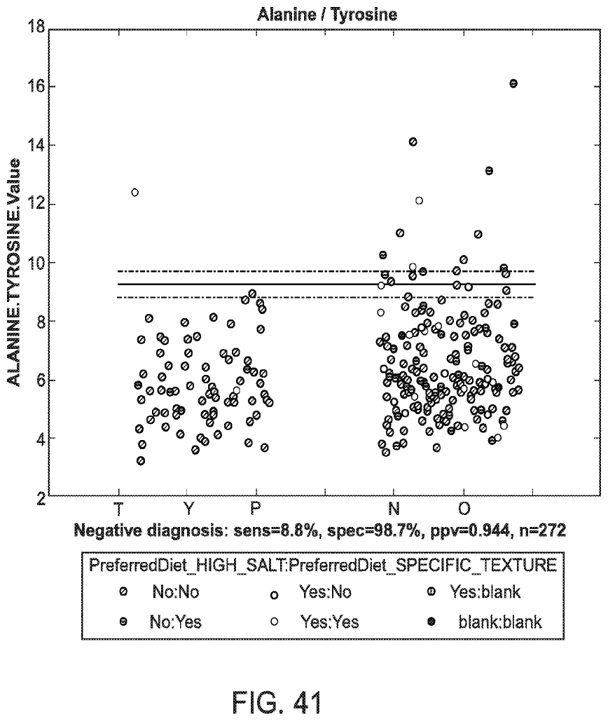

FIG. 41 is a graph showing ratios of concentrations of alanine to tyrosine obtained from the NeuroPoint diagnostic analysis of the 195 male subjects from the CAMP study. Subjects were from the following classes: red circles, no preference for high-salt diet or for specific texture; light blue circles, preference for high-salt diet but no preference for specific texture; green circles, preference for high-salt diet; dark blue circles, no preference for high-salt diet but preference for specific texture; yellow circles, preference for both high-salt diet and for specific texture; and purple, blank. Values from typically developing (TYP) subjects are shown on the left side of the graph, and values from not-typically developing (NOT) subject are shown on the right side of the graph.

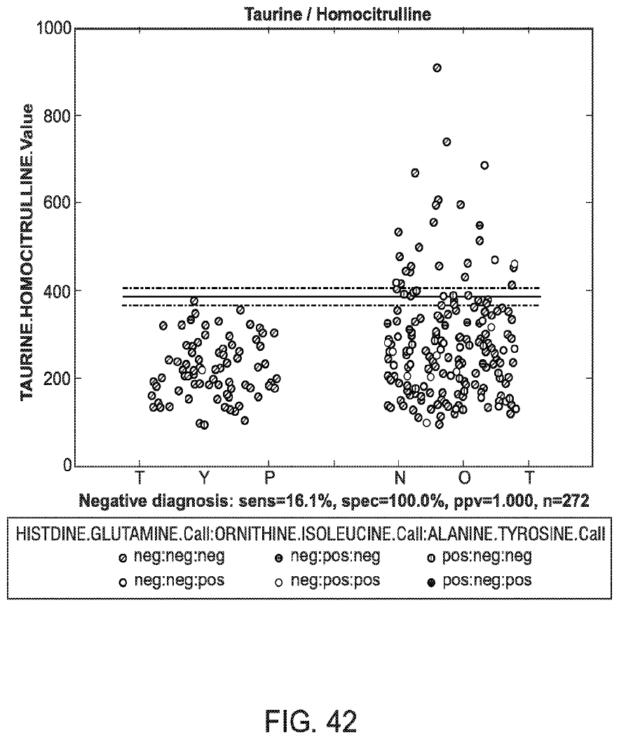

FIG. 42 is a graph showing ratios of concentrations of taurine to homocitrulline obtained from the NeuroPoint diagnostic analysis of the 195 male subjects from the CAMP study. Subjects were from the following classes: red circles, negative diagnoses based on ratios of histidine to glutamine, ornithine to isoleucine, and alanine to tyrosine; dark blue circles, negative diagnoses based on ratios of histidine to glutamine and alanine to tyrosine and positive diagnosis based on ratio of ornithine to isoleucine; green circles, positive diagnosis based on ratio of histidine to glutamine and negative diagnoses based on ratios of ornithine to isoleucine and alanine to tyrosine; light blue circles, negative diagnoses based on ratios of histidine to glutamine and ornithine to isoleucine and positive diagnosis based on ratio of alanine to tyrosine; yellow circles, negative diagnosis based on ratio of histidine to glutamine and positive diagnosis based on ratios of ornithine to isoleucine, and alanine to tyrosine; and purple circles, positive diagnoses based on ratios of histidine to glutamine and alanine to tyrosine and negative diagnosis based on ratio of ornithine to isoleucine. Values from typically developing (TYP) subjects are shown on the left side of the graph, and values from not-typically developing (NOT) subject are shown on the right side of the graph.

Multivariate analysis using selected amino acid ratio calls was performed.

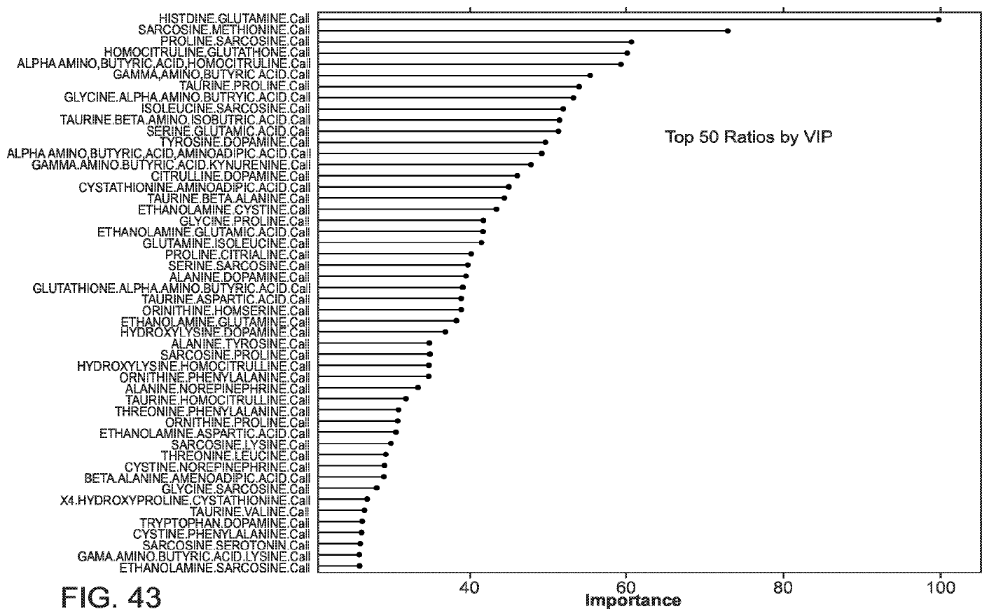

FIG. 43 is a graph showing the top 50 amino acid ratios according to the multivariate analysis.

The MIND I, MIND II, and ACHRI studies were performed to analyze metabolomics related to ASD. The demographics of subjects from the Banked Blood in MIND I, MIND II, and MIND III studies are provided in Table 21.

TABLE 21

| | MIND I | | MIND II | | MIND III | |
|---|---|---|---|---|---|---|
| | Typical | Autistic | Typical | Autistic | Typical | Autistic |
| Group size | 30 | 52 | 180 | 93 | 115 | 103 |
| Age (y) | 5.6 | 5.4 | 3.1 | 3 | 5.3 | 6 |
| Sex (male, %) | 87 | 79 | 69 | 83 | 69 | 83 |
| Developmental Status | 114 | 67 | 106 | 62 | — | — |

| | | | |
|---|---|---|---|
| Dietary Status | Fasted | Fed | Fasted |
| Anticoagulant | EDTA | Citrate | EDTA |

Data from the MIND I, MIND II, and ACHRI studies were used to identify metabolic subtypes with cross-study validation. The ACHRI study was used to identify subtypes using normalized features. Data from the MIND II study was used to evaluated ratios to identify features with predictive ratios.

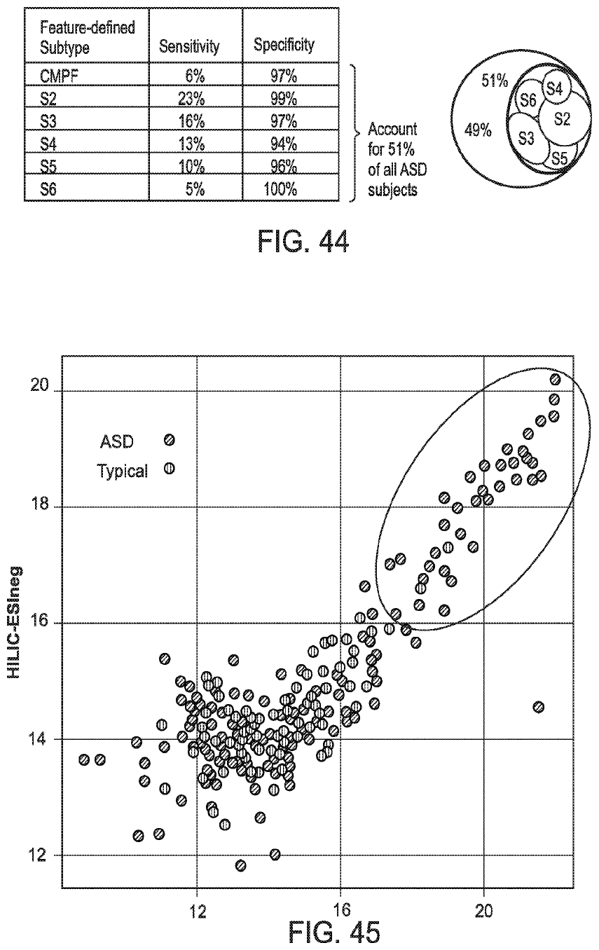

FIG. 44 is a flow chart illustrating the analysis of data from MIND II study.

FIG. 45 is a graph showing hydrophilic interaction liquid chromatography electrospray ionization-negative (HILIC-ESIneg) and C8 electrospray ionization-negative (C8-ESIneg) data from subjects in the MIND II study. Subjects were from the following classes: green circles, typically developing; red circles, ASD.

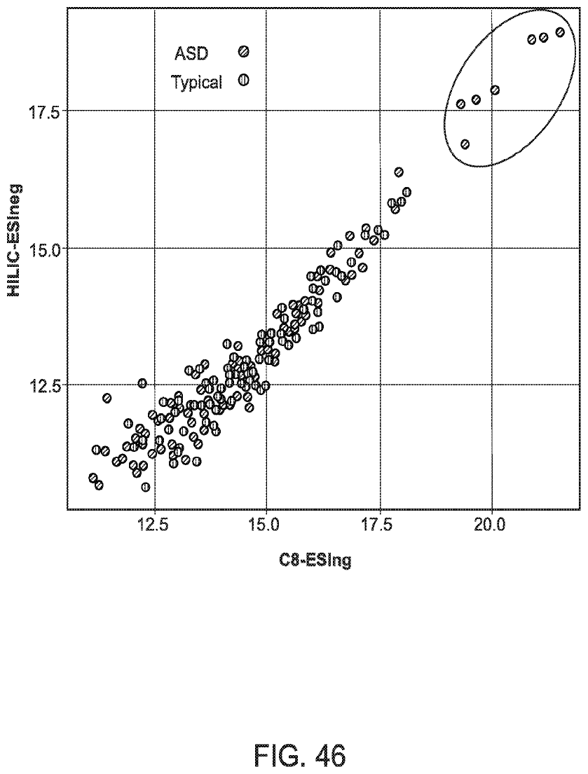

FIG. 46 is a graph showing hydrophilic interaction liquid chromatography electrospray ionization-negative (HILIC-ESIneg) and C8 electrospray ionization-negative (C8-ESIneg) data from subjects in the ACHRI study. Subjects were from the following classes: green circles, typically developing; red circles, ASD.

The relationship between levels of 3-carboxy-4-methyl-5-propyl-2-furanpropanoic acid (CMPF) and supplementation with vitamins or with fish oil was analyzed in subjects from the CAMP study.

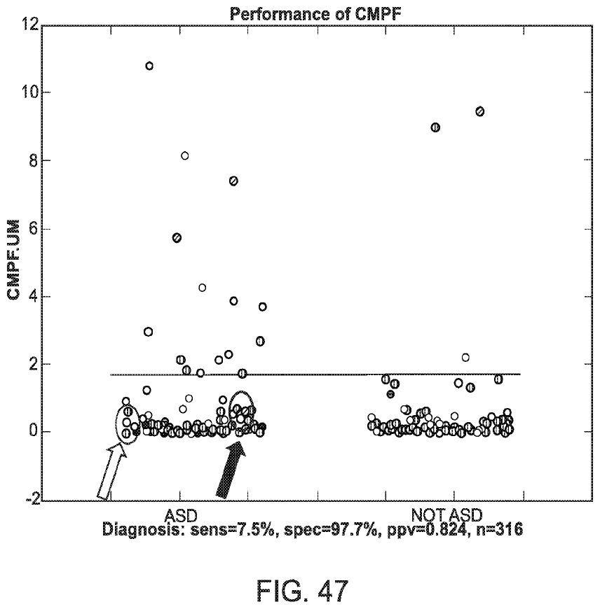

FIG. 47 is a graph showing concentrations of CMPF obtained from analysis of subjects from the CAMP study. Subjects were from the following classes: red circles, supplementation with both vitamins and fish oil; aqua circles, supplementation with fish oil only; blue circles, supplementation with vitamins only; green circles, no supplementation; yellow circles, blank.

The levels of the following biogenic amines were analyzed in subjects from the CAMP study: 3-methyl-L-histidine, 1-methyl-L-histidine, DL-B-amino-isobutyric acid, L-a-amino-n-butyric acid, B-alanine, L-alanine, L-arginine, L-asparagine, L-aspartic acid, L-carnosine, L-citrulline, ethanolamine, L-glutamic acid, L-glutamine, glycine, L-histidine, homoserine, hydroxy-L-proline, hydroxylysine, L-isoleucine, L-leucine, L-lysine, L-methionine, L-ornithine, L-phenylalanine, phosphoserine, L-proline, sarcosine, L-serine, taurine, L-threonine, L-tryptophan, L-tyrosine, L-valine, and y-amino-n-butyric acid.

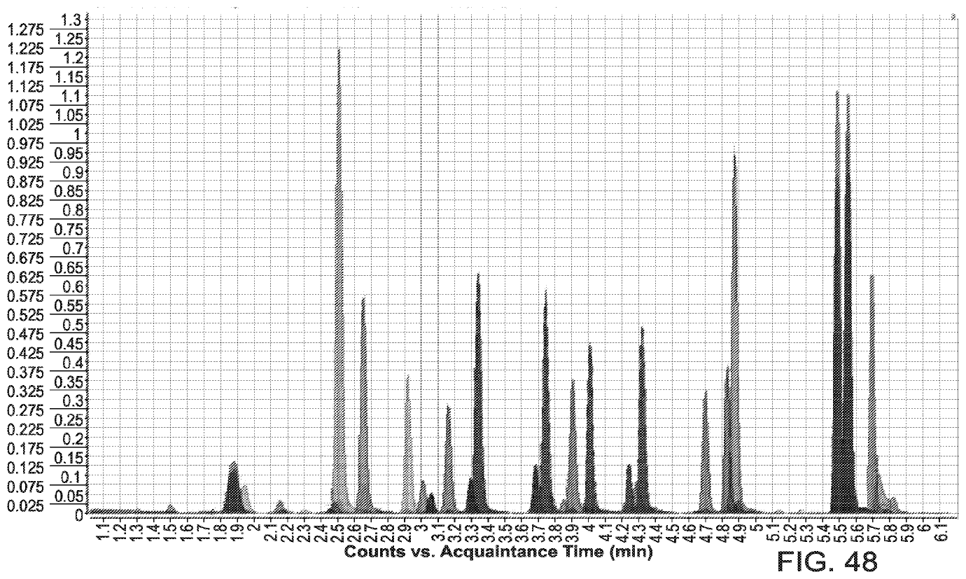

FIG. 48 is a graph showing mass spectrometric peaks in a sample following NeuroPoint diagnostic analysis.

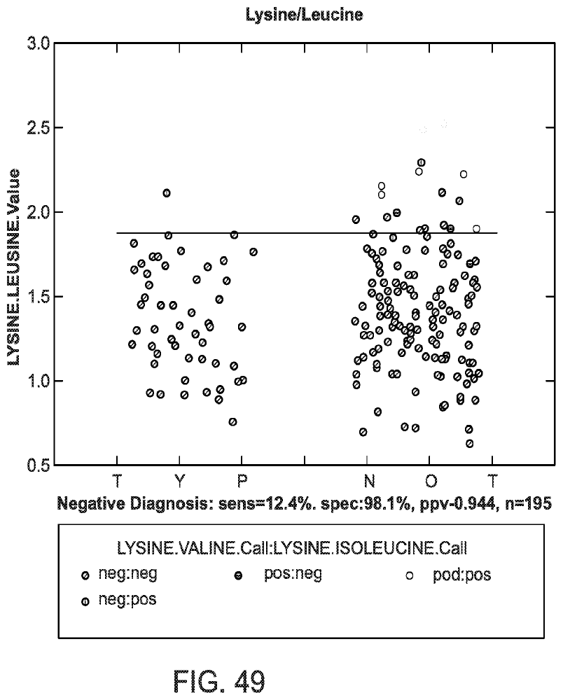

FIG. 49 is a graph showing ratios of concentrations of lysing to leucine obtained from the NeuroPoint diagnostic analysis of the 195 male subjects from the CAMP study. Subjects were from the following classes: red circles, negative for ratios of lysine to valine and lysine to isoleucine; dark blue circles, positive for ratio of lysine to valine and negative for ratio of lysine to isoleucine; yellow circles, positive for ratios of lysine to valine and lysine to isoleucine; and green circles, negative for ratio of lysine to valine and positive for ratio of lysine to isoleucine. Values from typically developing (TYP) subjects are shown on the left side of the graph, and values from not-typically developing (NOT) subject are shown on the right side of the graph.

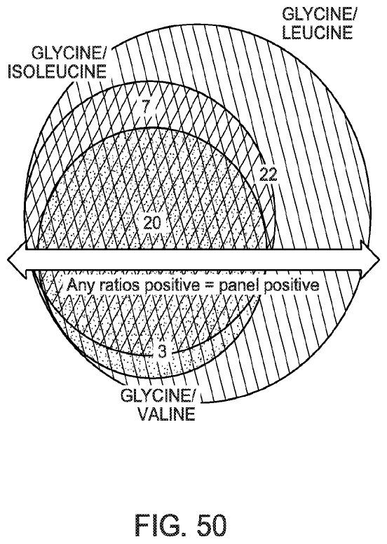

FIG. 50 is a Venn diagram showing relationship of subjects having positive scores based on ratios of concentrations of glycine to isoleucine, glycine to leucine, and glycine to valine.

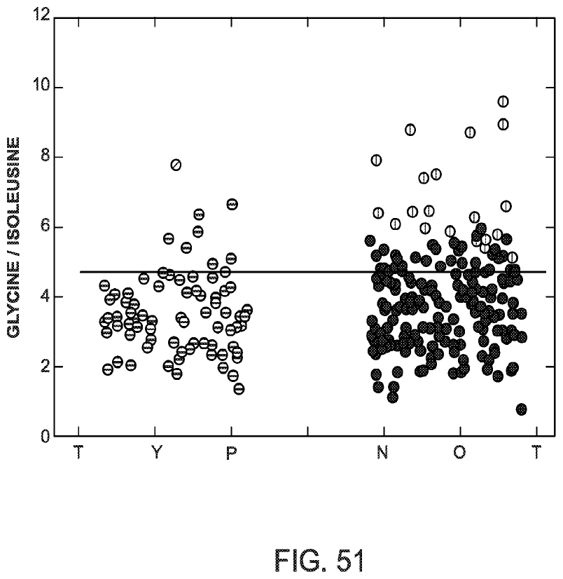

FIG. 51 is a graph showing ratios of concentrations of glycine to leucine obtained from the NeuroPoint diagnostic analysis of subjects from the CAMP study. Ratios from typically developing (TYP) subjects are shown on the left side of the graph, and ratios from not-typically developing (NOT) subject are shown on the right side of the graph.

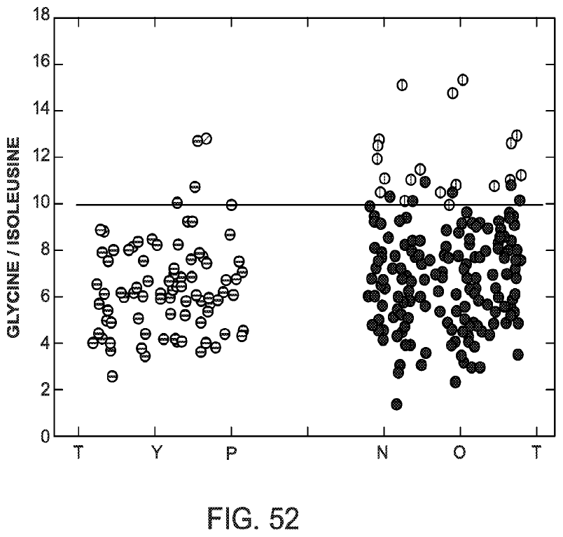

FIG. 52 is a graph showing ratios of concentrations of glycine to isoleucine obtained from the NeuroPoint diagnostic analysis of subjects from the CAMP study. Ratios from typically developing (TYP) subjects are shown on the left side of the graph, and ratios from not-typically developing (NOT) subject are shown on the right side of the graph.

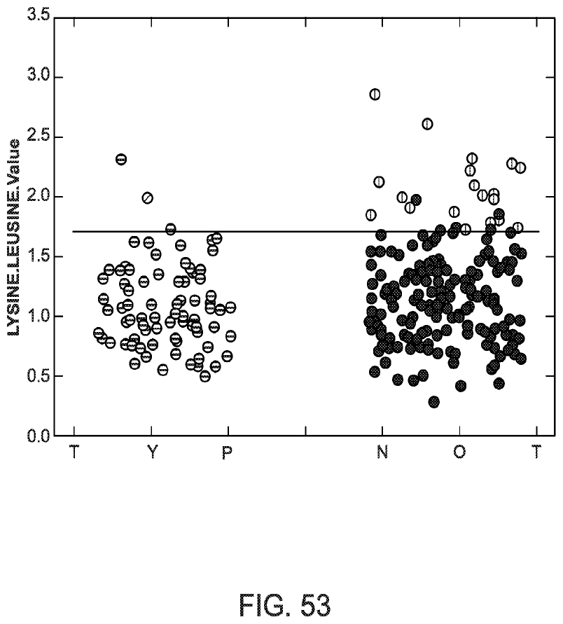

FIG. 53 is a graph showing ratios of concentrations of glycine to valine obtained from the NeuroPoint diagnostic analysis of subjects from the CAMP study. Ratios from typically developing (TYP) subjects are shown on the left side of the graph, and ratios from not-typically developing (NOT) subject are shown on the right side of the graph.

Metabotypes may include combinations or groups of ratios of concentrations of metabolites. Groups may include ratios in which a concentration of a first metabolite is compared to concentrations of various second metabolites. The concentration of the constant first metabolite may be in the numerator of the ratio and the concentrations of the various second metabolites may be in the denominator of the ratio. Alternatively, the concentration of the constant first metabolite may be in the denominator of the ratio and the concentrations of the various second metabolites may be in the numerator. The second metabolites within a group may have a common feature, or they be members of a common class of compounds. For example, the second analytes in such groups may be branched chain amino acids, hydrophobic amino acids, polar amino acids, negatively charged amino acids, positively charged amino acids, or metabolites in a common metabolic pathway, e.g., the citric acid cycle or fatty acid oxidation, single metabolite is used for the numerator and various metabolites are used for the denominator, or vice versa.

### Ratios of Concentrations

Additional ratios of metabolite concentrations that are indicative of ASD are provided in Table 22 and Table 23.

TABLE 22

| metabolite ratio | Metabolite 1 | Metabolite 2 | direction |
|---|---|---|---|
| ASPARAGINE_GLYCINE | L-Asparagine | L-Glycine | < |
| GLYCINE_ISOLEUCINE | L-Glycine | L-Isoleucine | > |
| GLYCINE_LEUCINE | L-Glycine | L-Leucine | > |
| GLYCINE_LYSINE | L-Glycine | L-Lysine | > |
| GLYCINE_PHENYLALANINE | L-Glycine | L-Phenylalanine | > |
| HISTIDINE_LEUCINE | L-Histidine | L-Leucine | > |
| KYNURENINE_ORNITHINE | Kynurenine | Ornithine | < |
| LYSINE_ORNITHINE | L-Lysine | Ornithine | < |
| XANTHINE_URIC.ACID | Xanthine | Uric Acid | > |
| XANTHINE_X4.HYDROXYPROLINE | Xanthine | 4-Hydroxyproline | > |

TABLE 23

| Test ID | direction | Metabolite 1 | Metabolite 2 |
|---|---|---|---|
| Lactic Acid_2.Deoxy.D.ribose | > | Lactic Acid | 2-Deoxy-D- |
| | | | ribose |
| Lactic Acid_3.Indoleacetic.acid | > | Lactic Acid | 3 Indoleacetic |
| | | | acid |
| Lactic Acid_D.Mannose | > | Lactic Acid | D-Mannose |
| Lactic Acid_GlycericAcid | > | Lactic Acid | GlycericAcid |
| Lactic Acid_L.Carnitine | > | Lactic Acid | L-Carnitine |
| Lactic Acid_L.Glutamine | > | Lactic Acid | L-Glutamine |
| Lactic Acid_L.Histidine | > | Lactic Acid | L-Histidine |
| Lactic Acid_L.Isoleucine | > | Lactic Acid | L-Isoleucine |
| Lactic Acid_L.Kynurenine | > | Lactic Acid | L-Kynurenine |
| Lactic Acid_L.Leucine | > | Lactic Acid | L-Leucine |
| Lactic Acid_L.Serine | > | Lactic Acid | L-Serine |
| Lactic Acid_L.Sorbose | > | Lactic Acid | L-Sorbose |
| Lactic Acid_L.Tyrosine | > | Lactic Acid | L-Tyrosine |
| Lactic Acid_N.Acetylglutamic.acid | > | Lactic Acid | N |
| | | | Acetylglutamic |
| | | | acid |
| Lactic Acid | > | Lactic Acid | NA |
| Lactic Acid_Phenylalanine | > | Lactic Acid | Phenylalanine |
| Lactic Acid_Phenylpyruvic.acid | > | Lactic Acid | Phenylpyruvic |
| | | | acid |
| Lactic Acid_Taurine | < | Lactic Acid | Taurine |
| Lactic Acid_UricAcid | > | Lactic Acid | UricAcid |
| alpha.Ketoglutaric.acid_Lactic Acid | < | alpha- | Lactic Acid |
| | | Ketoglutaric | |
| | | Acid | |
| alpha.Ketoglutaric.acid_3.Indoleacetic.acid | > | alpha- | 3 Indoleacetic |
| | | Ketoglutaric | acid |
| | | Acid | |
| alpha.Ketoglutaric.acid_beta.Nicotinamide.mononucleotide | > | alpha- | beta- |
| | | Ketoglutaric | Nicotinamide |
| | | Acid | Mononucleotide |
| alpha.Ketoglutaric.acid_Cellobiose | > | alpha- | Cellobiose |
| | | Ketoglutaric | |
| | | Acid | |
| alpha.Ketoglutaric.acid_D.Mannose | > | alpha- | D-Mannose |
| | | Ketoglutaric | |
| | | Acid | |
| alpha.Ketoglutaric.acid_GlycericAcid | > | alpha- | GlycericAcid |
| | | Ketoglutaric | |
| | | Acid | |
| alpha.Ketoglutaric.acid_Hypoxanthine | < | alpha- | Hypoxanthine |
| | | Ketoglutaric | |
| | | Acid | |
| alpha.Ketoglutaric.acid_L.Carnitine | > | alpha- | L-Carnitine |
| | | Ketoglutaric | |
| | | Acid | |
| alpha.Ketoglutaric.acid_L.Glutamine | > | alpha- | L-Glutamine |
| | | Ketoglutaric | |
| | | Acid | |
| alpha.Ketoglutaric.acid_L.Histidine | > | alpha- | L-Histidine |
| | | Ketoglutaric | |
| | | Acid | |
| alpha.Ketoglutaric.acid_L.Isoleucine | > | alpha- | L-Isoleucine |
| | | Ketoglutaric | |
| | | Acid | |
| alpha.Ketoglutaric.acid_L.Kynurenine | > | alpha- | L-Kynurenine |
| | | Ketoglutaric | |
| | | Acid | |
| alpha.Ketoglutaric.acid_L.Leucine | > | alpha- | L-Leucine |
| | | Ketoglutaric | |
| | | Acid | |
| alpha.Ketoglutaric.acid_L.Methionine | > | alpha- | L-Methionine |
| | | Ketoglutaric | |
| | | Acid | |
| alpha.Ketoglutaric.acid_L.Serine | > | alpha- | L-Serine |
| | | Ketoglutaric | |
| | | Acid | |
| alpha.Ketoglutaric.acid_L.Sorbose | > | alpha- | L-Sorbose |
| | | Ketoglutaric | |
| | | Acid | |
| alpha.Ketoglutaric.acid_L.Tyrosine | > | alpha- | L-Tyrosine |
| | | Ketoglutaric | |
| | | Acid | |
| alpha.Ketoglutaric.acid_Lipoamide | > | alpha- | Lipoamide |
| | | Ketoglutaric | |
| | | Acid | |
| alpha.Ketoglutaric.acid_myo.Inositol | > | alpha- | myo-Inositol |
| | | Ketoglutaric | |
| | | Acid | |
| alpha.Ketoglutaric.acid_N.Acetylneuraminic.acid | > | alpha- | N- |
| | | Ketoglutaric | Acetylneuraminic |
| | | Acid | Acid |
| alpha.Ketoglutaric.acid_N.carbamoyl.DL.aspartic.acid | < | alpha- | N-carbamoyL- |
| | | Ketoglutaric | DL-aspartic |
| | | Acid | acid |
| alpha.Ketoglutaric.acid_Phenylalanine | > | alpha- | Phenylalanine |
| | | Ketoglutaric | |
| | | Acid | |
| alpha.Ketoglutaric.acid_Pyruvic.acid | < | alpha- | Pyruvic acid |
| | | Ketoglutaric | |
| | | Acid | |
| alpha.Ketoglutaric.acid_Taurine | < | alpha- | Taurine |
| | | Ketoglutaric | |
| | | Acid | |
| alpha.Ketoglutaric.acid_UricAcid | > | alpha- | UricAcid |
| | | Ketoglutaric | |
| | | Acid | |
| alpha.Ketoglutaric.acid_Xanthine | < | alpha- | Xanthine |
| | | Ketoglutaric | |
| | | Acid | |
| alpha-Ketoglutaric acid | > | alpha- | NA |
| | | Ketoglutaric | |
| | | acid | |
| beta.Nicotinamide.mononucleotide_Lactic Acid | < | beta- | Lactic Acid |
| | | Nicotinamide | |
| | | Mononucleotide | |
| beta.Nicotinamide.mononucleotide_Cellobiose | < | beta- | Cellobiose |
| | | Nicotinamide | |
| | | Mononucleotide | |
| beta.Nicotinamide.mononucleotide_D.Maltose | < | beta- | D-Maltose |
| | | Nicotinamide | |
| | | Mononucleotide | |
| beta.Nicotinamide.mononucleotide_L.Glutamine | < | beta- | L-Glutamine |
| | | Nicotinamide | |
| | | Mononucleotide | |
| beta.Nicotinamide.mononucleotide_L.Isoleucine | < | beta- | L-Isoleucine |
| | | Nicotinamide | |
| | | Mononucleotide | |
| beta.Nicotinamide.mononucleotide_L.Tryptophan | < | beta- | L-Tryptophan |
| | | Nicotinamide | |
| | | Mononucleotide | |
| beta.Nicotinamide.mononucleotide_N.carbamoyl.DL.aspartic.acid | < | beta- | N-carbamoyL- |
| | | Nicotinamide | DL-aspartic |
| | | Mononucleotide | acid |
| beta.Nicotinamide.mononucleotide_Pyruvic.acid | < | beta- | Pyruvic acid |
| | | Nicotinamide | |
| | | Mononucleotide | |
| beta.Nicotinamide.mononucleotide_SuccinicAcid | < | beta- | SuccinicAcid |
| | | Nicotinamide | |
| | | Mononucleotide | |
| beta.Nicotinamide.mononucleotide_Taurine | < | beta- | Taurine |
| | | Nicotinamide | |
| | | Mononucleotide | |
| beta.Nicotinamide.mononucleotide_Xanthine | < | beta- | Xanthine |
| | | Nicotinamide | |
| | | Mononucleotide | |
| beta-Nicotinamide mononucleotide | < | beta- | NA |
| | | Nicotinamide | |
| | | mononucleotide | |
| Cellobiose_Lactic Acid | < | Cellobiose | Lactic Acid |
| Cellobiose_D.Mannose | > | Cellobiose | D-Mannose |
| Cellobiose_DL.2.Aminoadipic.acid | > | Cellobiose | DL-2 |
| | | | Aminoadipic |
| | | | acid |
| Cellobiose_GlycericAcid | > | Cellobiose | GlycericAcid |
| Cellobiose_L.Carnitine | > | Cellobiose | L-Carnitine |
| Cellobiose_L.Glutamine | > | Cellobiose | L-Glutamine |
| Cellobiose_L.Isoleucine | > | Cellobiose | L-Isoleucine |
| Cellobiose_L.Kynurenine | > | Cellobiose | L-Kynurenine |
| Cellobiose_L.Leucine | > | Cellobiose | L-Leucine |
| Cellobiose_L.Serine | > | Cellobiose | L-Serine |
| Cellobiose_L.Sorbose | > | Cellobiose | L-Sorbose |
| Cellobiose_N.Acetylglutamic.acid | > | Cellobiose | N |
| | | | Acetylglutamic |
| | | | acid |
| Cellobiose_N.Acetylneuraminic.acid | < | Cellobiose | N- |
| | | | Acetylneuraminic |
| | | | Acid |
| Cellobiose_N.carbamoyl.DL.aspartic.acid | < | Cellobiose | N-carbamoyL- |
| | | | DL-aspartic |
| | | | acid |
| Cellobiose | > | Cellobiose | NA |
| Cellobiose_Phenylalanine | > | Cellobiose | Phenylalanine |
| Cellobiose_Pyruvic.acid | < | Cellobiose | Pyruvic acid |
| Cellobiose_SuccinicAcid | < | Cellobiose | SuccinicAcid |
| Citramalic.acid_DL.2.Aminoadipic.acid | > | Citramalic | DL-2 |
| | | acid | Aminoadipic |
| | | | acid |
| Citramalic.acid_N.Acetylneuraminic.acid | < | Citramalic | N- |
| | | acid | Acetylneuraminic |
| | | | Acid |
| Cystine_GlycericAcid | > | Cystine | GlycericAcid |
| Cystine_L.Hydroxyglutaric.acid | > | Cystine | L- |
| | | | Hydroxyglutaric |
| | | | acid |
| D.Maltose_D.Mannose | > | D-Maltose | D-Mannose |
| D.Maltose_L.Carnitine | > | D-Maltose | L-Carnitine |
| D.Maltose_L.Isoleucine | > | D-Maltose | L-Isoleucine |
| D.Maltose_L.Kynurenine | > | D-Maltose | L-Kynurenine |
| D.Maltose_L.Sorbose | > | D-Maltose | L-Sorbose |
| D.Mannose_GlycericAcid | > | D-Mannose | GlycericAcid |
| D-Maltose | > | D-Maltose | NA |
| Galactonic.acid_Ketovaleric.acid | > | Galactonic | Ketovaleric |
| | | acid | acid |
| Galactonic.acid_L.Isoleucine | > | Galactonic | L-Isoleucine |
| | | acid | |
| GluconicAcid_Ketovaleric.acid | > | GluconicAcid | Ketovaleric |
| | | | acid |
| GluconicAcid_L.Isoleucine | > | GluconicAcid | L-Isoleucine |
| GluconicAcid_L.Leucine | > | GluconicAcid | L-Leucine |
| GlycericAcid_L.Malic.acid | < | GlycericAcid | L-Malic acid |
| GlycericAcid_N.Acetylneuraminic.acid | < | GlycericAcid | N- |
| | | | Acetylneuraminic |
| | | | Acid |
| Hypoxanthine_Lactic Acid | > | Hypoxanthine | Lactic Acid |
| Hypoxanthine_2.Deoxy.D.ribose | > | Hypoxanthine | 2-Deoxy-D- |
| | | | ribose |
| Hypoxanthine_3.Indoleacetic.acid | > | Hypoxanthine | 3 Indoleacetic |
| | | | acid |
| Hypoxanthine_Adipic.acid | > | Hypoxanthine | Adipic acid |
| Hypoxanthine_beta.Nicotinamide.mononucleotide | > | Hypoxanthine | beta- |
| | | | Nicotinamide |
| | | | Mononucleotide |
| Hypoxanthine_cis.Aconitic.acid | > | Hypoxanthine | cis-Aconitic |
| | | | acid |
| Hypoxanthine_Creatine | > | Hypoxanthine | Creatine |
| Hypoxanthine_Creatinine | > | Hypoxanthine | Creatinine |
| Hypoxanthine_Cystine | > | Hypoxanthine | Cystine |
| Hypoxanthine_D.Mannose | > | Hypoxanthine | D-Mannose |
| Hypoxanthine_DL.2.Aminoadipic.acid | > | Hypoxanthine | DL-2 |
| | | | Aminoadipic |
| | | | acid |
| Hypoxanthine_Galactonic.acid | > | Hypoxanthine | Galactonic acid |
| Hypoxanthine_GluconicAcid | > | Hypoxanthine | GluconicAcid |
| Hypoxanthine_GlycericAcid | > | Hypoxanthine | GlycericAcid |
| Hypoxanthine_Ketovaleric.acid | > | Hypoxanthine | Ketovaleric |
| | | | acid |
| Hypoxanthine_L.asparagine | > | Hypoxanthine | L-asparagine |
| Hypoxanthine_L.Carnitine | > | Hypoxanthine | L-Carnitine |
| Hypoxanthine_L.Glutamic.acid | > | Hypoxanthine | L-Glutamic |
| | | | acid |
| Hypoxanthine_L.Glutamine | > | Hypoxanthine | L-Glutamine |
| Hypoxanthine_L.Histidine | > | Hypoxanthine | L-Histidine |
| Hypoxanthine_L.Hydroxyglutaric.acid | > | Hypoxanthine | L- |
| | | | Hydroxyglutaric |
| | | | acid |
| Hypoxanthine_L.Isoleucine | > | Hypoxanthine | L-Isoleucine |
| Hypoxanthine_L.Kynurenine | > | Hypoxanthine | L-Kynurenine |
| Hypoxanthine_L.Leucine | > | Hypoxanthine | L-Leucine |
| Hypoxanthine_L.Methionine | > | Hypoxanthine | L-Methionine |
| Hypoxanthine_L.Serine | > | Hypoxanthine | L-Serine |
| Hypoxanthine_L.Sorbose | > | Hypoxanthine | L-Sorbose |
| Hypoxanthine_L.Threonine | > | Hypoxanthine | L-Threonine |
| Hypoxanthine_L.Tyrosine | > | Hypoxanthine | L-Tyrosine |
| Hypoxanthine_Lipoamide | > | Hypoxanthine | Lipoamide |
| Hypoxanthine_myo.Inositol | > | Hypoxanthine | myo-Inositol |
| Hypoxanthine_N.Acetylglutamic.acid | > | Hypoxanthine | N |
| | | | Acetylglutamic |
| | | | acid |
| Hypoxanthine_N.Acetylneuraminic.acid | > | Hypoxanthine | N- |
| | | | Acetylneuraminic |
| | | | Acid |
| Hypoxanthine | > | Hypoxanthine | NA |
| Hypoxanthine_Phenylalanine | > | Hypoxanthine | Phenylalanine |
| Hypoxanthine_Phenylpyruvic.acid | > | Hypoxanthine | Phenylpyruvic |
| | | | acid |
| Hypoxanthine_Salicylic.acid | > | Hypoxanthine | Salicylic acid |
| Hypoxanthine_SuccinicAcid | > | Hypoxanthine | SuccinicAcid |
| Hypoxanthine_trans.Aconitic.acid | > | Hypoxanthine | trans Aconitic |
| | | | acid |
| Hypoxanthine_UricAcid | > | Hypoxanthine | Uric Acid |
| Hypoxanthine_Uridine | > | Hypoxanthine | Uridine |
| Hypoxanthine_Xanthine | > | Hypoxanthine | Xanthine |
| L.asparagine_Cystine | < | L-asparagine | Cystine |
| L.asparagine_GlycericAcid | > | L-asparagine | GlycericAcid |
| L.asparagine_L.Carnitine | > | L-asparagine | L-Carnitine |
| L.Aspartic.Acid_Citramalic.acid | > | L-Aspartic | Citramalic acid |
| | | Acid | |
| L.Aspartic.Acid_DL.2.Aminoadipic.acid | > | L-Aspartic | DL-2 |
| | | Acid | Aminoadipic |
| | | | acid |
| L.Aspartic.Acid_L.Carnitine | > | L-Aspartic | L-Carnitine |
| | | Acid | |
| L.Aspartic.Acid_L.Isoleucine | > | L-Aspartic | L-Isoleucine |
| | | Acid | |
| L.Aspartic.Acid_L.Sorbose | > | L-Aspartic | L-Sorbose |
| | | Acid | |
| L.Carnitine_L.Histidine | < | L-Carnitine | L-Histidine |
| L.Carnitine_L.Malic.acid | < | L-Carnitine | L-Malic acid |
| L.Glutamic.acid_Adipic.acid | > | L-Glutamic | Adipic acid |
| | | acid | |
| L.Glutamic.acid_L.Carnitine | > | L-Glutamic | L-Carnitine |
| | | acid | |
| L.Glutamic.acid_L.Hydroxyglutaric.acid | > | L-Glutamic | L- |
| | | acid | Hydroxyglutaric |
| | | | acid |
| L.Histidine_UricAcid | > | L-Histidine | UricAcid |
| L.Hydroxyglutaric.acid_N.Acetylneuraminic.acid | < | L- | N- |
| | | Hydroxyglutaric | Acetylneuraminic |
| | | acid | Acid |
| L.Isoleucine_N.Acetylneuraminic.acid | < | L-Isoleucine | N- |
| | | | Acetylneuraminic |
| | | | Acid |
| L.Malic.acid_UricAcid | > | L-Malic acid | UricAcid |
| L.Methionine_L.Isoleucine | > | L-Methionine | L-Isoleucine |
| L.Sorbose_N.Acetylneuraminic.acid | < | L-Sorbose | N- |
| | | | Acetylneuraminic |
| | | | Acid |
| L.Tryptophan_Adipic.acid | > | L-Tryptophan | Adipic acid |
| L.Tryptophan_Cystine | < | L-Tryptophan | Cystine |
| L.Tryptophan_DL.2.Aminoadipic.acid | > | L-Tryptophan | DL-2 |
| | | | Aminoadipic |
| | | | acid |
| L.Tryptophan_GlycericAcid | > | L-Tryptophan | GlycericAcid |
| L.Tryptophan_L.Isoleucine | > | L-Tryptophan | L-Isoleucine |
| L.Tryptophan_L.Leucine | > | L-Tryptophan | L-Leucine |
| L-asparagine | > | L-asparagine | NA |
| L-Aspartic Acid | > | L-Aspartic | NA |
| | | Acid | |
| L-Glutamic acid | > | L-Glutamic | NA |
| | | acid | |
| myo.Inositol_Lactic Acid | < | myo-Inositol | Lactic Acid |
| myo.Inositol_N.Acetylneuraminic.acid | < | myo-Inositol | N- |
| | | | Acetylneuraminic |
| | | | Acid |
| myo-Inositol | < | myo-Inositol | NA |
| N.Acetylglutamic.acid_N.Acetylneuraminic.acid | < | N | N- |
| | | Acetylglutamic | Acetylneuraminic |
| | | acid | Acid |
| N.carbamoyl.DL.aspartic.acid_Lactic Acid | > | N-carbamoyL- | Lactic Acid |
| | | DL-aspartic | |
| | | acid | |
| N.carbamoyl.DL.aspartic.acid_Adipic.acid | > | N-carbamoyL- | Adipic acid |
| | | DL-aspartic | |
| | | acid | |
| N.carbamoyl.DL.aspartic.acid_cis.Aconitic.acid | > | N-carbamoyL- | cis-Aconitic |
| | | DL-aspartic | acid |
| | | acid | |
| N.carbamoyl.DL.aspartic.acid_Creatine | > | N-carbamoyL- | Creatine |
| | | DL-aspartic | |
| | | acid | |
| N.carbamoyl.DL.aspartic.acid_Creatinine | > | N-carbamoyL- | Creatinine |
| | | DL-aspartic | |
| | | acid | |
| N.carbamoyl.DL.aspartic.acid_D.Mannose | > | N-carbamoyL- | D-Mannose |
| | | DL-aspartic | |
| | | acid | |
| N.carbamoyl.DL.aspartic.acid_D.pantothenic.acid | > | N-carbamoyL- | D-pantothenic |
| | | DL-aspartic | acid |
| | | acid | |
| N.carbamoyl.DL.aspartic.acid_DL.2.Aminoadipic.acid | > | N-carbamoyL- | DL-2 |
| | | DL-aspartic | Aminoadipic |
| | | acid | acid |
| N.carbamoyl.DL.aspartic.acid_DL.Isocitric.acid | > | N-carbamoyL- | DL-Isocitric |
| | | DL-aspartic | acid |
| | | acid | |
| N.carbamoyl.DL.aspartic.acid_GluconicAcid | > | N-carbamoyL- | GluconicAcid |
| | | DL-aspartic | |
| | | acid | |
| N.carbamoyl.DL.aspartic.acid_GlycericAcid | > | N-carbamoyL- | GlycericAcid |
| | | DL-aspartic | |
| | | acid | |
| N.carbamoyl.DL.aspartic.acid_Ketovaleric.acid | > | N-carbamoyL- | Ketovaleric |
| | | DL-aspartic | acid |
| | | acid | |
| N.carbamoyl.DL.aspartic.acid_L.asparagine | > | N-carbamoyL- | L-asparagine |
| | | DL-aspartic | |
| | | acid | |
| N.carbamoyl.DL.aspartic.acid_L.Serine | > | N-carbamoyL- | L-Serine |
| | | DL-aspartic | |
| | | acid | |
| N.carbamoyl.DL.aspartic.acid_L.Sorbose | > | N-carbamoyL- | L-Sorbose |
| | | DL-aspartic | |
| | | acid | |
| N.carbamoyl.DL.aspartic.acid_L.Threonine | > | N-carbamoyL- | L-Threonine |
| | | DL-aspartic | |
| | | acid | |
| N.carbamoyl.DL.aspartic.acid_L.Tryptophan | > | N-carbamoyL- | L-Tryptophan |
| | | DL-aspartic | |
| | | acid | |
| N.carbamoyl.DL.aspartic.acid_L.Tyrosine | > | N-carbamoyL- | L-Tyrosine |
| | | DL-aspartic | |
| | | acid | |
| N.carbamoyl.DL.aspartic.acid_N.Acetylglutamic.acid | > | N-carbamoyL- | N |
| | | DL-aspartic | Acetylglutamic |
| | | acid | acid |
| N.carbamoyl.DL.aspartic.acid_Phenylalanine | > | N-carbamoyL- | Phenylalanine |
| | | DL-aspartic | |
| | | acid | |
| N.carbamoyl.DL.aspartic.acid_Phenylpyruvic.acid | > | N-carbamoyL- | Phenylpyruvic |
| | | DL-aspartic | acid |
| | | acid | |
| N.carbamoyl.DL.aspartic.acid_Salicylic.acid | > | N-carbamoyL- | Salicylic acid |
| | | DL-aspartic | |
| | | acid | |
| N.carbamoyl.DL.aspartic.acid_trans.Aconitic.acid | > | N-carbamoyL- | trans Aconitic |
| | | DL-aspartic | acid |
| | | acid | |
| N.carbamoyl.DL.aspartic.acid_UricAcid | > | N-carbamoyL- | UricAcid |
| | | DL-aspartic | |
| | | acid | |
| N-carbamoyl-DL-aspartic acid | > | N-carbamoyl- | NA |
| | | DL-aspartic | |
| | | acid | |
| Phenylalanine_Cystine | < | Phenylalanine | Cystine |
| Pyruvic.acid_2.Deoxy.D.ribose | > | Pyruvic acid | 2-Deoxy-D- |
| | | | ribose |
| Pyruvic.acid_Adipic.acid | > | Pyruvic acid | Adipic acid |
| Pyruvic.acid_Cystine | > | Pyruvic acid | Cystine |
| Pyruvic.acid_D.Mannose | > | Pyruvic acid | D-Mannose |
| Pyruvic.acid_D.pantothenic.acid | > | Pyruvic acid | D-pantothenic |
| | | | acid |
| Pyruvic.acid_DL.2.Aminoadipic.acid | > | Pyruvic acid | DL-2 |
| | | | Aminoadipic |
| | | | acid |
| Pyruvic.acid_Galactonic.acid | > | Pyruvic acid | Galactonic acid |
| Pyruvic.acid_GluconicAcid | > | Pyruvic acid | GluconicAcid |
| Pyruvic.acid_GlycericAcid | > | Pyruvic acid | GlycericAcid |
| Pyruvic.acid_Ketovaleric.acid | > | Pyruvic acid | Ketovaleric |
| | | | acid |
| Pyruvic.acid_L.Carnitine | > | Pyruvic acid | L-Carnitine |
| Pyruvic.acid_L.Glutamic.acid | > | Pyruvic acid | L-Glutamic |
| | | | acid |
| Pyruvic.acid_L.Hydroxyglutaric.acid | > | Pyruvic acid | L- |
| | | | Hydroxyglutaric |
| | | | acid |
| Pyruvic.acid_L.Isoleucine | > | Pyruvic acid | L-Isoleucine |
| Pyruvic.acid_L.Kynurenine | > | Pyruvic acid | L-Kynurenine |
| Pyruvic.acid_L.Leucine | > | Pyruvic acid | L-Leucine |
| Pyruvic.acid_L.Sorbose | > | Pyruvic acid | L-Sorbose |
| Pyruvic.acid_L.Tryptophan | > | Pyruvic acid | L-Tryptophan |
| Pyruvic.acid_L.Tyrosine | > | Pyruvic acid | L-Tyrosine |
| Pyruvic.acid_Phenylalanine | > | Pyruvic acid | Phenylalanine |
| Pyruvic.acid_Phenylpyruvic.acid | > | Pyruvic acid | Phenylpyruvic |
| | | | acid |
| Pyruvic.acid_Salicylic.acid | > | Pyruvic acid | Salicylic acid |
| Pyruvic.acid_UricAcid | > | Pyruvic acid | UricAcid |
| SuccinicAcid_3.Indoleacetic.acid | > | SuccinicAcid | 3 Indoleacetic |
| | | | acid |
| SuccinicAcid_D.Mannose | > | SuccinicAcid | D-Mannose |
| SuccinicAcid_GlycericAcid | > | SuccinicAcid | GlycericAcid |
| SuccinicAcid_L.Carnitine | > | SuccinicAcid | L-Carnitine |
| SuccinicAcid_L.Histidine | > | SuccinicAcid | L-Histidine |
| SuccinicAcid_L.Hydroxyglutaric.acid | > | SuccinicAcid | L- |
| | | | Hydroxyglutaric |
| | | | acid |
| SuccinicAcid_L.Isoleucine | > | SuccinicAcid | L-Isoleucine |
| SuccinicAcid_L.Kynurenine | > | SuccinicAcid | L-Kynurenine |
| SuccinicAcid_L.Methionine | > | SuccinicAcid | L-Methionine |
| SuccinicAcid_L.Sorbose | > | SuccinicAcid | L-Sorbose |
| SuccinicAcid_L.Tyrosine | > | SuccinicAcid | L-Tyrosine |
| SuccinicAcid_myo.Inositol | > | SuccinicAcid | myo-Inositol |
| SuccinicAcid | > | SuccinicAcid | NA |
| SuccinicAcid_Taurine | < | SuccinicAcid | Taurine |
| SuccinicAcid_UricAcid | > | SuccinicAcid | UricAcid |
| Taurine_2.Deoxy.D.ribose | > | Taurine | 2-Deoxy-D- |
| | | | ribose |
| Taurine_Adipic.acid | > | Taurine | Adipic acid |
| Taurine_cis.Aconitic.acid | > | Taurine | cis-Aconitic |
| | | | acid |
| Taurine_Citramalic.acid | > | Taurine | Citramalic acid |
| Taurine_Ketovaleric.acid | > | Taurine | Ketovaleric |
| | | | acid |
| Taurine_L.Carnitine | > | Taurine | L-Carnitine |
| Taurine_L.Histidine | > | Taurine | L-Histidine |
| Taurine_L.Isoleucine | > | Taurine | L-Isoleucine |
| Taurine_L.Kynurenine | > | Taurine | L-Kynurenine |
| Taurine_L.Sorbose | > | Taurine | L-Sorbose |
| Taurine_N.Acetylglutamic.acid | > | Taurine | N |
| | | | Acetylglutamic |
| | | | acid |
| Taurine_Phenylalanine | > | Taurine | Phenylalanine |
| Taurine_Salicylic.acid | > | Taurine | Salicylic acid |
| Taurine_trans.Aconitic.acid | > | Taurine | trans Aconitic |
| | | | acid |
| Taurine_UricAcid | > | Taurine | Uric Acid |
| X2.Deoxy.D.ribose_DL.2.Aminoadipic.acid | > | X2-Deoxy-D- | DL-2 |
| | | ribose | Aminoadipic |
| | | | acid |
| X2.Deoxy.D.ribose_L.Isoleucine | > | X2-Deoxy-D- | L-Isoleucine |
| | | ribose | |
| Xanthine_2.Deoxy.D.ribose | > | Xanthine | 2-Deoxy-D- |
| | | | ribose |
| Xanthine_Adipic.acid | > | Xanthine | Adipic acid |
| Xanthine_cis.Aconitic.acid | > | Xanthine | cis-Aconitic |
| | | | acid |
| Xanthine_Creatine | > | Xanthine | Creatine |
| Xanthine_DL.2.Aminoadipic.acid | > | Xanthine | DL-2 |
| | | | Aminoadipic |
| | | | acid |
| Xanthine_GlycericAcid | > | Xanthine | GlycericAcid |
| Xanthine_Ketovaleric.acid | > | Xanthine | Ketovaleric |
| | | | acid |
| Xanthine_L.Carnitine | > | Xanthine | L-Carnitine |
| Xanthine_L.Isoleucine | > | Xanthine | L-Isoleucine |
| Xanthine_trans.Aconitic.acid | > | Xanthine | trans Aconitic |
| | | | acid |
| Xanthine_UricAcid | > | Xanthine | UricAcid |

### Example 8: Sample Test Report

A sample test report is provided below.

### Patient and Sample Data

The following information about the patient and sample is provided: patient's name, patient's date of birth, patient's sex, specimen type (e.g., plasma), date of specimen collection, date specimen was received, test panel used, and date and time of test report.

### Results Summary

The algorithmic analysis indicates patient has form(s) of amino acid dysregulation associated with autism spectrum disorder (ASD). Specific details of findings are listed below.

Metabotype 3: An imbalance between the plasma concentrations of Glycine and Asparagine was detected. This imbalance includes above average Glycine

Metabotype 5: An imbalance between the plasma concentrations of Glycine and Isoleucine was detected. This imbalance includes above average Glycine and below average Isoleucine.

Metabotype 11: An imbalance between the plasma concentrations of Glycine and branched chain amino acids (BCAA) was detected. This imbalance includes above average Glycine and below average BCAA.

Additional Findings: Level of individual amines indicates 1 is out of range. The following analytes were tested: Alanine, Arginine, Asparagine, Aspartic Acid, β-Alanine, β-Aminoisobutyric Acid, Citrulline, Ethanolamine, γ-Aminoisobutyric Acid, Glutamic Acid, Glutamine, Glycine, Histidine, Homocitrulline, Homoserine, Isoleucine, Kynurenine, Leucine, Lysine, Methionine, Ornithine, Phenylalanine, Proline, Sarcosine, Serine, Serotonin, Taurine, Threonine, Tryptophan, Tyrosine, Valine, and 4-Hydroxyproline. Levels of individual analytes are provided in Table 24.

TABLE 24
Amine Results Table: Individual measurements
of analytes by LC-MS/MS

| # | Analyte | Normal Range (μM) | Result | Flag |
|---|---|---|---|---|
| 1 | Alanine | 173-360 | 242 | Normal |
| 2 | Arginine | 53-91 | 74 | Normal |
| 3 | Asparagine | 31-48 | 39 | Normal |
| 4 | Aspartic Acid | 1.8-4.1 | 2.1 | Normal |
| 5 | β-Alanine | 2.9-6.5 | 5.9 | Normal |
| 6 | β-Aminoisobutyric Acid | 1.0-3.7 | 2.4 | Normal |
| 7 | Citrulline | 20-35 | 21 | Normal |
| 8 | Ethanolamine | 5.1-8.4 | 6.1 | Normal |
| 9 | γ-Aminoisobutyric Acid | .21-.37 | 0.42 | Normal |
| 10 | Glutamic Acid | 22-65 | 23 | Normal |
| 11 | Glutamine | 423-608 | 589 | Normal |
| 12 | Glycine | 151-280 | 181 | Normal |
| 13 | Histidine | 63-85 | 92 | HIGH |
| 14 | Homocitrulline | .18-.40 | 0.21 | Normal |
| 15 | Homoserine | .11-.16 | 0.85 | Normal |
| 16 | Isoleucine | 39-66 | 57 | Normal |
| 17 | Kynurenine | 1.4-2.5 | 1.9 | Normal |
| 18 | Leucine | 73-120 | 92 | Normal |
| 19 | Lysine | 103-155 | 148 | Normal |
| 20 | Methionine | 14-24 | 19 | Normal |
| 21 | Ornithine | 24-47 | 41 | Normal |
| 22 | Phenylalanine | 40-55 | 52 | Normal |
| 23 | Proline | 90-190 | 91 | Normal |
| 24 | Sarcosine | .80-1.8 | 1.5 | Normal |
| 25 | Serine | 81-127 | 102 | Normal |
| 26 | Serotonin | .05-.36 | 0.54 | Normal |
| 27 | Taurine | 27-55 | 33 | Normal |
| 28 | Threonine | 60-121 | 97 | Normal |
| 29 | Tryptophan | 46-76 | 52 | Normal |
| 30 | Tyrosine | 41-76 | 71 | Normal |
| 31 | Valine | 152-267 | 231 | Normal |
| 32 | 4-Hydroxproline | 15-31 | 28 | Normal |

### Exemplary Guidance

Recommend follow up with neurodevelopment/ASD specialist for further evaluation. Some studies indicate dietary modification may be beneficial for patients with metabolic dysregulation.

### Example 9: Metabotypes Indicative of Altered Purine Degradation

Metabotypes indicative of altered purine degradation were identified. Plasma metabolites in CAMP subjects were measured by quantitative LC-MS/MS, and statistical analysis of metabolites was used to identify metabotypes. Samples were divided into a training set and an independent validation set, i.e, test set. The following metabolites were analyzed: xanthine, hypoxanthine, inosine, uric acid, and taurine. Hemolyzed samples with hemoglobin levels >200 mg/dL were excluded from analysis due to interference.

A single reproducible metabotype was identified in 6.3% of CAMP ASD subjects as shown in Table 25.

TABLE 25

| | Sensitivity | | Specificity | | PPV | |
|---|---|---|---|---|---|---|
| Ratio | Train | Test | Train | Test | Train | Test |
| xanthine/urate | 0.044 | 0.083 | 1.000 | 0.979 | 1.000 | 0.900 |

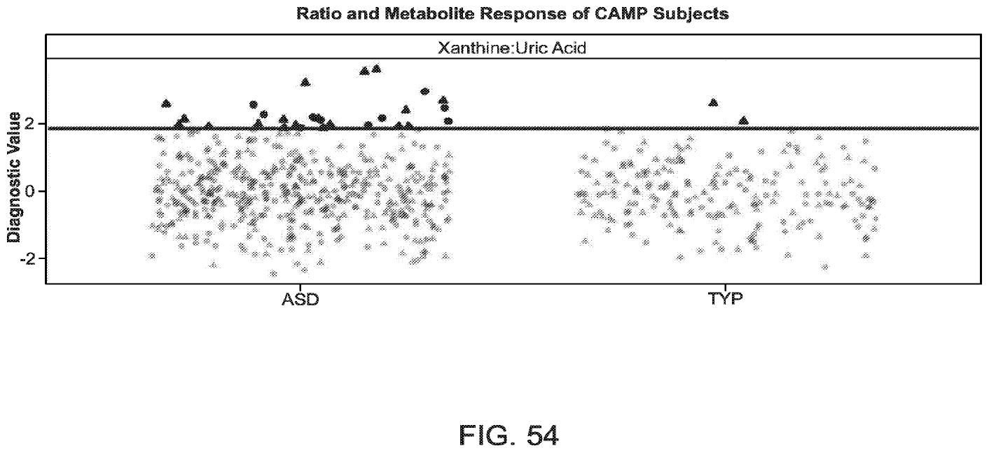

FIG. 54 is a graph showing diagnostic value of ratios of concentrations of xanthine to uric acid obtained from diagnostic analysis of subjects from the CAMP study. Circles represent data from training set, and triangles represent data from test set. Data from ABD subjects is shown on the left, and data from typically developing subjects is shown on the right.

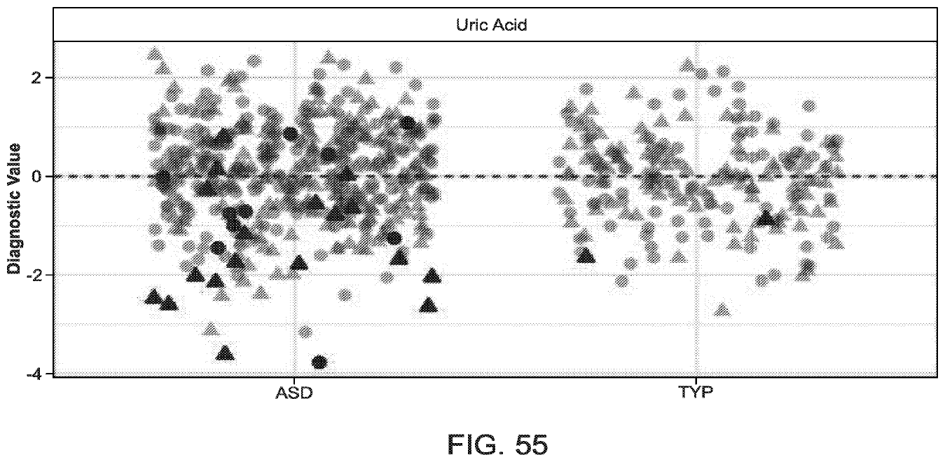

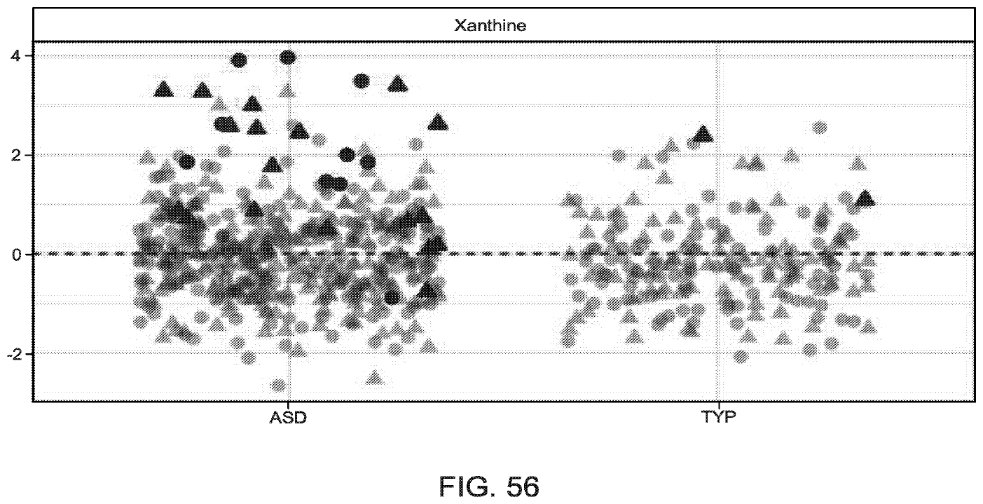

FIG. 55 is a graph showing diagnostic value of concentrations of uric acid obtained from diagnostic analysis of subjects from the CAMP study. Circles represent data from training set, and triangles represent data from test set. Data from ABD subjects is shown on the left, and data from typically developing subjects is shown on the right.

FIG. 56 is a graph showing diagnostic value of concentrations of xanthine obtained from diagnostic analysis of subjects from the CAMP study. Circles represent data from training set, and triangles represent data from test set. Data from ABD subjects is shown on the left, and data from typically developing subjects is shown on the right.

Elevated xanthine is correlated with taurine (p=. 64), an amine which reduces XOR activity and is evaluated in molybdenum cofactor required by XOR (MOCOS) deficiency. The data suggest that biomarkers of defective sulfite metabolism may provide a link to understanding the biology in a subset of children with ASD. The data further suggest that altered activity of purine degradation is associated with a subset of ASD subjects and that the xanthine to uric acid metabotype can be used to identify individuals who belong to this subset.

### Example 10: Metabotypes Indicative of Altered Energy Homeostasis

Metabotypes indicative of altered energy homeostasis were identified. Plasma metabolites in CAMP subjects were measured by quantitative LC-MS/MS, and statistical analysis of metabolites was used to identify metabotypes. Samples were divided into a training set and an independent validation set, i.e, test set. The following metabolites were analyzed: α-ketoglutarate, lactate, pyruvate, succinate, alanine, and phenylalanine. Lactate, pyruvate, and alanine are commonly used to assess mitochondrial bioenergetic function.

22.3% of CAMP ASD subjects were identified by an energy related metabotype. Results are summarized in Table 26.

TABLE 26

| | Sensitivity | | Specificity | | PPV | |
|---|---|---|---|---|---|---|
| Ratio | Train | Test | Train | Test | Train | Test |
| α-ketoglutarate/lactate | 0.074 | 0.134 | 0.981 | 0.981 | 0.905 | 0.946 |
| α-ketoglutarate/alanine | 0.070 | 0.061 | 0.981 | 0.991 | 0.900 | 0.941 |
| lactate/alanine | 0.051 | 0.073 | 0.990 | 0.991 | 0.929 | 0.950 |
| lactate/phenylalanine | 0.058 | 0.084 | 0.990 | 0.981 | 0.938 | 0.917 |

The data suggest that altered activity of mitochondrial energy homeostasis pathways is associated with a subset of ASD subjects and that specific metabotypes can be used to identify individuals who belong to this subset.

### Example 11: Metabotypes Indicative of Altered Amine Metabolism

Metabotypes indicative of altered amine metabolism were identified. Plasma metabolites in CAMP subjects were measured by quantitative LC-MS/MS, and statistical analysis of metabolites was used to identify metabotypes. Samples were divided into a training set and an independent validation set, i.e, test set. Thirty-two amine-containing metabolites were analyzed.

Five amine metabotypes identified 21.5% of CAMP ASD subjects. Results are summarized in Table 27.

TABLE 27

| | Sensitivity | | Specificity | | PPV | |
|---|---|---|---|---|---|---|
| Ratio | Train | Test | Train | Test | Train | Test |
| asparagine/glycine | 0.069 | 0.073 | 0.990 | 0.990 | 0.947 | 0.950 |
| glycine/phenylalanine | 0.062 | 0.065 | 1.000 | 0.990 | 1.000 | 0.944 |
| histidine/leucine | 0.088 | 0.084 | 0.980 | 0.980 | 0.920 | 0.917 |
| kynurenine/ornithine | 0.050 | 0.057 | 0.990 | 0.990 | 0.929 | 0.938 |
| lysine/ornithine | 0.065 | 0.050 | 0.990 | 0.990 | 0.944 | 0.929 |

Altered neurotransmission, mitochondrial biology, nitrogen metabolism may be associated with amine metabotypes.

The data suggest that altered activity of pathways involved in one or more of amine metabolism, neurotransmission, and neurotransmitter synthesis are associated with a subset of ASD subjects and that specific metabotypes can be used to identify individuals who belong to this subset.

### Example 12: Summary and Conclusion

Overall, 41% of CAMP ASD subjects were identified in the training and test sets. Metabotype positive subjects that share multiple metabotypes may have more complex metabolic phenotypes, i.e., may have alterations in multiple metabolic pathways. Conversely, subjects identified by a single amine, purine, or energy process metabotype may have a specific metabolic dysregulation.

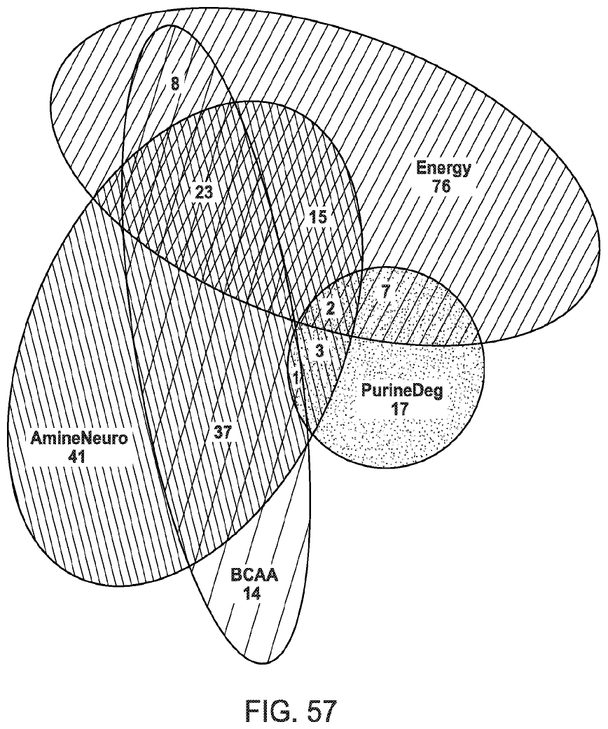

FIG. 57 is a Venn diagram showing the number of subjects having alterations in various metabolic pathways.

Data from the large ASD CAMP cohort indicate that metabolomic analysis allows detection of reproducible metabotypes with a prevalence >5%. Biological processes, including mitochondrial biology/energy metabolism, amino acid metabolism and homeostasis, and purine catabolism were associated metabotypes were identified in 41% of CAMP ASD subjects. Thus, the results demonstrate that subsets of ASD are associated with alterations of specific metabolic pathways and that such pathways can be identified through metabolomic analysis.

### REFERENCES

1. Association AP (2013): Diagnostic and statistical manual of mental disorders (5th ed.). Arlington, VA: American Psychiatric Publishing.
2. Autism, Developmental Disabilities Monitoring Network Surveillance Year Principal I, Centers for Disease C, Prevention (2012): Prevalence of autism spectrum disorders—Autism and Developmental Disabilities Monitoring Network, 14 sites, United States, 2008. MMWR Surveill Summ. 61:1-19.
3. El Achkar C M, Spence S J (2015): Clinical characteristics of children and young adults with co-occurring autism spectrum disorder and epilepsy. Epilepsy Behav. 47:183-190.
4. Spence S J, Schneider M T (2009): The role of epilepsy and epileptiform EEGs in autism spectrum disorders. Pediatr Res. 65:599-606.
5. Kerns C M, Kendall P C, Berry L, Souders M C, Franklin M E, Schultz R T, et al. (2014): Traditional and atypical presentations of anxiety in youth with autism spectrum disorder. J Autism Dev Disord. 44:2851-2861.
6. Buie T, Campbell D B, Fuchs G J, 3rd, Furuta G T, Levy J, Vandewater J, et al. Evaluation, diagnosis, and treatment of gastrointestinal disorders in individuals with ASDs: a consensus report. Pediatrics. 125 Suppl 1:S1-18.
7. Bourgeron T (2016): Current knowledge on the genetics of autism and propositions for future research. C R Biol. 339:300-307.
8. Sandin S, Lichtenstein P, Kuja-Halkola R, Hultman C, Larsson H, Reichenberg A (2017): The Heritability of Autism Spectrum Disorder. JAMA. 318:1182-1184.
9. Geschwind D H, State M W (2015): Gene hunting in autism spectrum disorder: on the path to precision medicine. Lancet Neurol. 14:1109-1120.
10. Sanders S J, He X, Willsey A J, Ercan-Sencicek A G, Samocha K E, Cicek A E, et al. (2015): Insights into Autism Spectrum Disorder Genomic Architecture and Biology from 71 Risk Loci. Neuron. 87:1215-1233.
11. Mandy W, Lai M C (2016): Annual Research Review: The role of the environment in the developmental psychopathology of autism spectrum condition. J Child Psychol Psychiatry. 57:271-292.
12. Lord C, Risi S, Lambrecht L, Cook E H, Jr., Leventhal B L, DiLavore P C, et al. (2000): The autism diagnostic observation schedule-generic: a standard measure of social and communication deficits associated with the spectrum of autism. J Autism Dev Disord. 30:205-223.
13. Lord C, Risi S, DiLavore P S, Shulman C, Thurm A, Pickles A (2006): Autism from 2 to 9 years of age. Arch Gen Psychiatry. 63:694-701.
14. Autism, Developmental Disabilities Monitoring Network Surveillance Year Principal I, Centers for Disease C, Prevention (2009): Prevalence of autism spectrum disorders—Autism and Developmental Disabilities Monitoring Network, United States, 2006. MMWR Surveill Summ. 58:1-20.
15. Dawson G (2010): Recent advances in research on early detection, causes, biology, and treatment of autism spectrum disorders. Curr Opin Neurol. 23:95-96.
16. Dawson G, Rogers S, Munson J, Smith M, Winter J, Greenson J, et al. (2010): Randomized, controlled trial of an intervention for toddlers with autism: the Early Start Denver Model. Pediatrics. 125:e17-23.
17. Ganz M L (2007): The lifetime distribution of the incremental societal costs of autism. Arch Pediatr Adolesc Med. 161:343-349.
18. McPartland J C (2017): Developing Clinically Practicable Biomarkers for Autism Spectrum Disorder. J Autism Dev Disord. 47:2935-2937.
19. Hazlett H C, Gu H, Munsell B C, Kim S H, Styner M, Wolff J J, et al. (2017): Early brain development in infants at high risk for autism spectrum disorder. Nature. 542:348-351.
20. Shen M D, Kim S H, Mckinstry R C, Gu H, Hazlett H C, Nordahl C W, et al. (2017): Increased Extra-axial Cerebrospinal Fluid in High-Risk Infants Who Later Develop Autism. Biol Psychiatry. 82:186-193.
21. West P R, Amaral D G, Bais P, Smith A M, Egnash L A, Ross M E, et al. (2014): Metabolomics as a tool for discovery of biomarkers of autism spectrum disorder in the blood plasma of children. PLOS One. 9:e112445.
22. Beger R D D W, Schmidt M A, Gross S S, Kirwan J A, Cascante M, Brennan L., Wishart D S O M, Hankemeier T, Broadhurst D I, Lane A N, Suhre K, Kastenmüller, G S S, Thiele I, Fiehn O, Kaddurah-Daouk R (2016): Metabolomics enables precision medicine: “A White Paper, Community Perspective”. Metabolomics. 12:149.
23. Żurawicz E. Kałużna-Czaplińska J (2015): Analysis of amino acids in autism spectrum disorders. TrAC Trends in Analytical Chemistry. 73:91-118.
24. Novarino G, El-Fishawy P. Kayserili H, Meguid N A, Scott E M, Schroth J, et al. (2012): Mutations in BCKD-kinase lead to a potentially treatable form of autism with epilepsy. Science. 338:394-397.
25. Tarlungeanu D C, Deliu E, Dotter C P, Kara M, Janiesch P C, Scalise M, et al. (2016): Impaired Amino Acid Transport at the Blood Brain Barrier Is a Cause of Autism Spectrum Disorder. Cell. 167:1481-1494 e1418.
26. Riedl A, Gieger C, Hauner H, Daniel H, Linseisen J (2017): Metabotyping and its application in targeted nutrition: an overview. Br J Nutr. 117:1631-1644.
27. Benjamini Y, and Hochberg, Y. (1995): Controlling the False Discovery Rate: A Practical and Powerful Approach to Multiple Testing Journal of the Royal Statistical Society Series B. 57:289-300.
28. Suzuki R, Shimodaira H (2006): Pvclust: an R package for assessing the uncertainty in hierarchical clustering. Bioinformatics. 22:1540-1542.
29. Stacklies W R H, Scholz M, Walther D, Selbig J. (2007): pcaMethods—a Bioconductor package providing PCA methods for incomplete data. Bioinformatics. 23:1164-1167.
30. Eastman J W, Sherwin J E, Wong R, Liao C L, Currier R J, Lorey F, et al. (2000): Use of the phenylalanine:tyrosine ratio to test newborns for phenylketonuria in a large public health screening programme. J Med Screen. 7:131-135.
31. Garcia-Cazorla A, Oyarzabal A, Fort J, Robles C, Castejon E, Ruiz-Sala P, et al. (2014): Two novel mutations in the BCKDK (branched-chain keto-acid dehydrogenase kinase) gene are responsible for a neurobehavioral deficit in two pediatric unrelated patients. Hum Mutat. 35:470-477.
32. Zheng H F, Wang W Q, Li X M, Rauw G, Baker G B (2017): Body fluid levels of neuroactive amino acids in autism spectrum disorders: a review of the literature. Amino Acids. 49:57-65.
33. Park Y, Reyna-Neyra A, Philippe L, Thoreen C C (2017): mTORC1 Balances Cellular Amino Acid Supply with Demand for Protein Synthesis through Post-transcriptional Control of ATF4. Cell Rep. 19:1083-1090.
34. Maynard T M, Manzini M C (2017): Balancing Act: Maintaining Amino Acid Levels in the Autistic Brain. Neuron. 93:476-479.
35. Scholl-Burgi S, Haberlandt E, Heinz-Erian P, Deisenhammer F, Albrecht U, Sigl S B, et al. (2008): Amino acid cerebrospinal fluid/plasma ratios in children: influence of age, gender, and antiepileptic medication. Pediatrics. 121:e920-926.
36. Gray N, Zia R, King A, Patel V C, Wendon J, McPhail M J, et al. (2017): High-Speed Quantitative UPLC-M S Analysis of Multiple Amines in Human Plasma and Serum via Precolumn Derivatization with 6-Aminoquinolyl-N-hydroxysuccinimidyl Carbamate: Application to Acetaminophen-Induced Liver Failure. Anal Chem. 89:2478-2487.
37. Flahault A C M, Thomas G (2005): Sample size calculation should be performed for design accuracy in diagnostic test studies. J Clin Epidemiol. 58:859-862.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

What is claimed is:

1. A method of providing guidance for treating a subject that has or is at risk of developing a neurodevelopmental disorder, the method comprising:
receiving results of an assay in which concentrations of at least two metabolites are measured in a sample from a subject that has or is at risk of developing a neurodevelopmental disorder, the results comprising at least one ratio of concentrations of the at least two metabolites, a reference level that provides an indication as to whether the at least one ratio is imbalanced, and identification of a metabolic pathway comprising at least one of the at least two metabolites; and
based on the results, providing guidance for treating the subject that has or is suspected of having a neurodevelopmental disorder.

2. The method of claim 1, wherein the metabolic pathway is selected from the group consisting of an amine metabolic pathway, a metabolic pathway related to a gut microbiome, a mitochondrial energy homeostasis pathway, a neurotransmission pathway, a neurotransmitter synthesis pathway, a purine degradation pathway, and a reactive oxidative species metabolic pathway.

3. The method of claim 1, wherein the neurodevelopmental disorder is an autism spectrum disorder.

4. The method claim 1, wherein the at least two metabolites are selected from the group consisting of alanine, asparagine, aspartic acid, glycine, histidine, hypoxanthine, inosine, kynurenine, lactate, leucine, lysine, ornithine, phenylalanine, pyruvate, succinate, taurine, uric acid, xanthine, and α-ketoglutarate.

5. The method of claim 1, wherein the at least one ratio is selected from the group consisting of asparagine to glycine; glycine to phenylalanine; histidine to leucine; kynurenine to ornithine; lactate to alanine; lactate to phenylalanine; lysine to ornithine; xanthine to uric acid; α-ketoglutarate to alanine; and α-ketoglutarate to lactate.

6. The method of claim 2, wherein:
the metabolic pathway is purine degradation; and
the at least two metabolites are selected from the group consisting of hypoxanthine, inosine, taurine, uric acid, and xanthine.

7. The method of claim 6, wherein the at least one ratio comprises xanthine to uric acid.

8. The method of claim 2, wherein:
the metabolic pathway is a mitochondrial energy homeostasis pathway; and
the at least two metabolites are selected from the group consisting of alanine, lactate, phenylalanine, pyruvate, succinate, and α-ketoglutarate.

9. The method of claim 8, wherein the at least one ratio comprises α-ketoglutarate to alanine; α-ketoglutarate to lactate, lactate to alanine; or lactate to phenylalanine.

10. The method of claim 2, wherein:
the metabolic pathway is an amine metabolic pathway, a neurotransmission pathway, or a neurotransmitter synthesis pathway; and
the at least two metabolites are selected from the group consisting of asparagine, glycine, histidine, kynurenine, leucine, lysine, ornithine, and phenylalanine.

11. The method of claim 2, wherein the at least one ratio comprises asparagine to glycine;
glycine to phenylalanine; histidine to leucine; kynurenine to ornithine; or lysine to ornithine.

12. The method of claim 1, wherein the reference population comprises a subset of autism spectrum disorder (ASD) subjects.

13. The method of claim 1, wherein the reference population comprises typically developing subjects.

14. The method of claim 1, wherein the sample is a plasma sample.

15. The method of claim 1, further comprising
receiving the sample from the subject; and
performing the assay.

16. The method of claim 15, wherein the assay comprises mass spectrometry.

17. The method of claim 1, wherein the results additional data about the subject.

18. The method of claim 17, wherein the additional data comprises one selected from the group consisting of medical history of the subject, medical history of a family member of the subject, and genetic data from the subject.

19. The method of claim 1, wherein the guidance comprises a recommendation selected from the group consisting of a dietary modification, a drug, a medical grade food, and a supplement.

20. The method of claim 1, wherein the guidance comprises a recommendation to consult with a neurodevelopment specialist.
